SEQUENCE LISTING (327 sequences)

SEQ ID NO: 1 - protein, 190 aa, Homo sapiens
QGWQAFKNDATEIIPELGEYPEPPPELENNKTMNRAENGGRPPHHPFETKDVSEYSCRELHFTRYVTDGPCRSAKPVTELVCSGQCGPARLLPNAIGRGKWWRPSGPDFRCIPDRYRAQRVQLLCPGGEAPRARKVRLVASCKCKRLTRFHNQSELKDFGTEAARPQKGRKPRPRARSAKANQAELENAY

SEQ ID NO: 2 - protein, 106 aa, Mus musculus
TDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 324 aa, Mus musculus
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 4 - protein, 132 aa, Mus musculus
METDTILLWVLLLWVPGSTGDIVLTQSPASLAVSLGQRATISCKASQSVDYAGDNYMNWYQQKPGQPPKLLIYTASNLESGIPARFSGSGSGTDFTLNIHPVEEEDAATYYCQQSNEDPPTFGGGTKLELKR

SEQ ID NO: 5 - DNA, 393 bp, Mus musculus
atggagacagacacaatcctgctatgggtgctgctgctctgggttccaggctccactggtgacattgtgctgacccaatctccagcttctttggctgtgtctctagggcagagggccaccatctcctgcaaggccagccaaagtgttgattatgctggtgataattatatgaactggtaccaacagaaaccaggacagccacccaaactcctcatctatactgcatccaatctagagtctgggatcccagccaggtttagtggcagtgggtctgggacagacttcaccctcaacattcatcctgtggaggaggaggatgctgcaacctattactgtcagcaaagtaatgaggatcctccgacgttcggtggaggcaccaagttggagctcaaa

SEQ ID NO: 6 - protein, 138 aa, Mus musculus
MGWNWIFILILSVTTGVHSEVQLQQSGPELVKPGASVKISCKASGYPFTGYFMHWVKQSPENSLEWIGEINPSTGGTTYNQRFKGKATLTVDKSSSTAYMQLKSLTSEDSAVYYCTRWGYNPYALDYWGQGTSVTVSS

SEQ ID NO: 7 - DNA, 411 bp, Mus musculus
atgggatggaactggatctttattttaatcctgtcagtaactacaggtgtccactctgaggtccaactgcagcagtctggacctgagctggtgaagcctggggcttcagtgaagatatcctgcaaggcttctggttacccattcactggctacttcatgcactgggtgaaacaaagtcctgaaaatagtcttgagtggattggagagattaatcctagcactgggggtactacctacaaccagaggttcaagggcaaggccacattaactgtagataaatcctccagcacagcctacatgcagctcaagagcctgacatctgaagactctgcagtctattactgtacaagatggggatataacccctatgctttggactactggggtcaaggaacctcagtcaccgtctcg

SEQ ID NO: 8 - protein, 127 aa, Mus musculus
MRPSIQFLGLLLFWLHGAQCDIQMTQSPSSLSASLGGKVTITCKASQDINKYIAWYQHKPGKGPRLLIHYTSTLQPGIPSRFSGSGSGRDYSFSISNLEPEDIATYYCLQYDNLYTFGGGTKLELKR

SEQ ID NO: 9 - DNA, 378 bp, Mus musculus
atgagaccgtctattcagttcctggggctcttgttgttctggcttcatggtgctcagtgtgacatccagatgacacagtctccatcctcactgtctgcatctctgggaggcaaagtcaccatcacttgcaaggcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatctatacacgttcggaggggggaccaagctggaactgaaa

SEQ ID NO: 10 - protein, 134 aa, Mus musculus
MEWSWVFLFLLSVTAGVHSQVQLQQSGAELMKPGASVKISCKATGYTFSSYWIEWVKQRPGHGPEWIGEIFPRNGSTYYNEKFKGKATFTADTSSNTAYMQLSSLTSDDSAVYYCAIINTLDYWGQGTTLTVSS

SEQ ID NO: 11 - DNA, 399 bp, Mus musculus
atggaatggagctgggtctttctcttcctcctgtcagtaactgcaggtgtccactcccaggttcagctgcagcagtctggagctgagctgatgaagcctggggcctcagtgaagatatcctgcaaggctactggctacacattcagtagttattggatagagtgggtaaagcagaggcctggacatggccctgagtggattggagagatttttcctagaaatggtagtacttactacaatgagaaattcaagggcaaggccacattcactgcagatacatcctccaacacagcctacatgcaactcagcagcctgacatctgacgactctgccgtctattactgtgcaattattaatacgcttgactactggggccaaggcaccactctcacagtctcg

SEQ ID NO: 12 - protein, 132 aa, Mus musculus
MKLPVRLLVLMFWIPVSSSDVVMTQTPLSLPVSLGDQASISCRSSQSLVHSNGDTYLHWYLQKPGQSPKLLIYEISNRFSGVPDRFSGSGSGTDFTLKISRVEAEDLGVYFCSQSTHVPFTFGGGTKLEIKR

SEQ ID NO: 13 - DNA, 393 bp, Mus musculus
atgaagttgcctgttaggctgttggtgctgatgttctggattcctgtttccagtagtgatgttgtgatgacccaaactccactctccctgcctgtcagtcttggagatcaagcctccatctcttgcagatctagtcagagccttgtacacagtaatggagacacctatttacattggtacctacagaagccaggccagtctccaaaactcctgatctacgaaatttccaaccgattttctggggtcccagacaggttcagtggcagtggatcagggacagatttcacactcaagatcagcagagtggaggctgaggatctgggagtttatttctgctctcaaagtacacatgttccgttcacgttcggaggggggaccaagctggaaataaaa

SEQ ID NO: 14 - protein, 135 aa, Mus musculus
MGWSWVIFFLMAVVIGINSEVQLQQSGAELVRSGASVRLSCTASGFNIKDYYMHWVRQRPEQGLEWIGWNDPETGDTEYAPKFQGKATMTSDTSSNTAYLQLSSLTSEDTAVYYCNEGSGLIPYWGPGTLVTVSS

SEQ ID NO: 15 - DNA, 402 bp, Mus musculus
atgggatggagctgggtcatcttcttcctgatggcagtggttataggaatcaattcagaggttcagctgcagcagtctggggcagaacttgtgaggtcaggggcctcagtcaggttgtcctgcacagcttctggcttcaacattaaagactactatatgcactgggtgaggcagcggcctgaacagggcctggagtggattggatggaatgatcctgagactggtgatactgaatatgccccgaagttccagggcaaggccactatgacttcagacacatcctccaacacagcctacctgcagctcagcagcctgacatctgaggacactgccgtctattactgtaatgagggctcgggcttgattccttactggggcccagggactctggtcacagtctcg

SEQ ID NO: 16 - protein, 132 aa, Mus musculus
MKLPVRLLVLMFWIPVSSSDIVMTQTPLSLSVTPGQPASISCRSSQSLVHSNGDTYLHWYLQKPGQPPQLLIYEISNRFSGVPDRFSGSGSGTDFTLKISRVEAEDVGVYYCSQSTHVPFTFGQGTKLEIKR

SEQ ID NO: 17 - DNA, 396 bp, Mus musculus
atgaagttgcctgttaggctgttggtgctgatgttctggattcctgtttccagtagtgatatcgtgatgacccagactccactctccctgtctgtcactccgggtcaaccggcctccatctcttgccgttctagtcagagccttgtacacagtaatggtgacacctatttacattggtacttacagaagccaggccagcctccacaactcctgatctacgaaatttccaaccgcttttctggggtcccagaccgtttcagtggcagtggttcagggacagatttcacactcaagatcagccgcgtggaggctgaggatgtgggtgtttattactgctctcaaagtacacatgttccgttcacgttcggtcaggggaccaagctggaaattaaacgt

SEQ ID NO: 18 - protein, 135 aa, Mus musculus
MKCSWVIFFLMAVVIGITSQVQLVQSGAEVKKPGSSVKVSCKASGFNIKDYYMHWVRQAPGQGLEWMGWNDPETGDTEYAPKFQGRVTITADESTNTAYMELSSLRSEDTAVYYCNEGSGLIPYWGQGTLVTVSS

SEQ ID NO: 19 - DNA, 405 bp, Mus musculus
atgaaatgcagctgggtcatcttcttcctgatggcagtggttataggaatcacttcacaggttcagctggtccagtctggggcagaggttaaaaagccagggtcctcagtcaaagtgtcctgcaaggcttctggcttcaacattaaagactactatatgcactgggtgagacaggcgcctggtcagggcctggagtggatgggatggaatgatcctgagactggtgatactgaatatgccccgaagttccagggcagggtcactattactgcagacgaatccaccaacacagcctacatggagctctcatccctgcgttctgaggacactgccgtctattactgtaatgagggctcgggcttgattccttactggggccaagggactctggtcaccgtctcgagc

SEQ ID NO: 20 - protein, 132 aa, Mus musculus
MKLPVRLLVLMFWIPVSSSDVVLTQTPLSLPVSLGDQASISCRSSQSLVHSNGDVYLHWYLQKPGQSPKLLIYEVSNRFSGVPDRFSGSGSGTDFTLKISRVEAEDLGVYFCSQSTHVPFTFGGGTKLEIKR

SEQ ID NO: 21 - DNA, 393 bp, Mus musculus
atgaagttgcctgttaggctgttggtgctgatgttctggattcctgtttccagcagtgatgttgtattgacccaaactccactctccctgcctgtcagtcttggagatcaagcctccatctcttgtagatctagtcagagccttgtacacagtaatggagacgtctatttacattggtacctacagaagccaggccagtctccaaagctcctgatctacgaagtttccaaccgattttctggggtcccagacaggttcagtggcagtggatcagggacagatttcacactcaagatcagcagagtggaggctgaggatctgggagtttatttctgctctcaaagtacacatgttccgttcacgttcggaggggggaccaagctggaaataaaa

SEQ ID NO: 22 - protein, 135 aa, Mus musculus; identical to SEQ ID NO: 14

SEQ ID NO: 23 - DNA, 402 bp, Mus musculus
atgggatggagctgggtcatcttcttcctgatggcagtggttataggaatcaattcagaggttcagctgcagcagtctgggacagagcttgtgaggtcaggggcctcagtcaagttggcctgcacagcttctggcttcaacattaaaaactactatatgcactgggtgaggcagaggcctgaacagggcctggagtggattggatggattgatcctgagaatggtgatactgagtatgccccgaagttccaggacaaggccactatgactgcagacacatcctccaacacagcctacctgcagctcagcagcctgacatctgaggacactgccgtctattactgtaatgagggctcgggcttgattccttactggggcccagggactctggtcactgtctcg

SEQ ID NO: 24 - protein, 126 aa, Mus musculus
MKLPVRLLVLMFWIPASSSDIVMTQSHKFMSTSVGGRVSITCKASQDVDTSVAWYQQKPGQAPKLLVYWASTRHTGVPDRFTGSGSGTDFTLTISNVQSEDLADYFCQQYSNYPTFGAGTKLELKR

SEQ ID NO: 25 - DNA, 393 bp, Mus musculus
atgaagttgcctgttaggctgttggtgctgatgttctggattcctgcttccagcagtggtgttgtgatgacccaaactccactctccctgcctgtcagtcttggagatcaagcctccatctcgtgcagatctagtcagagccttgttcacagtaatggaaacacctatttatattggtacctacagaagccaggccagtctccaaagctcctgctcttcaaagtttccacccgattttctggggtcccagacaggttcagtggcagtggatcagggacagatttcacactcaagatcaccagagtagaggctgaggatctgggaatttatttctgctctcaaagttcacatattcctccgacgttcggtggaggcaccaagttggagctcaaa

SEQ ID NO: 26 - protein, 133 aa, Mus musculus
MDWLWTLLFLMAAAQSAQAQIQLVQSGPELKKPGETVKISCKASGYTVTDYGMNWVKQSPGKDLKWMGWIDTYTEKPTYADDFKGRFAFSLETSASTAYLQISNLKNEDSAAYFCLRSNFDFWGQGTTLTVSS

SEQ ID NO: 27 - DNA, 396 bp, Mus musculus
atggattggctgtggaccttgctattcctgatggcagctgcccaaagtgcccaagcacagatccagttggtgcagtctggacctgagctgaagaagcctggagagacagtcaagatctcctgcaaggcttctgggtataccgtcacagactatggaatgaactgggtgaagcagagtccaggaaaggatttaaagtggatgggctggatagacacctacactgaaaagccaacatatgctgatgacttcaagggacgatttgccttctctttggaaacctctgccagcactgcctacttgcagatcagcaacctcaaaaatgaggactcggctgcatatttctgtctaagatccaattttgacttttggggccaaggcaccactctcacagtctcg

SEQ ID NO: 28 - protein, 127 aa, Mus musculus
MESQIQVFVYMLLWLSGVEGDIVMTQSHKFMSTSVGGRVSITCKASQDVDTSVAWYQQKPGQAPKLLVYWASTRHTGVPDRFTGSGSGTDFTLTISNVQSEDLADYFCQQYSNYPTFGAGTKLELKR

SEQ ID NO: 29 - DNA, 378 bp, Mus musculus
atggagtcacagattcaggtctttgtatacatgttgctgtggttgtctggtgttgaaggagacattgtgatgacccagtctcacaaattcatgtccacatcagtaggaggcagggtcagcatcacctgcaaggccagtcaggatgtggatacttctgtagcctggtatcaacagaaaccagggcaagctcctaaacttctggtttactgggcatccacccgccacactggagtccctgatcgcttcacaggcagtggatctgggacagatttcactctcaccattagcaatgtgcagtctgaagacttggcagattatttctgtcagcaatatagcaactatcccacgttcggtgctgggaccaagctggaactgaaa

SEQ ID NO: 30 - protein, 139 aa, Mus musculus
MKCSWVIFFLMAVVTGVNSEVQLQQSGAELVRPGASVKLSCKASGFNIKDYYMHGVKHRPEQGLEWIGRIDPENGNTIYDPKFQGKATITADTSSNTAYLQLSSLTSEDTAVYYCARSPYDYHAWFAYWGQGTLVTVSS

SEQ ID NO: 31 - DNA, 414 bp, Mus musculus
atgaaatgcagctgggtcatcttcttcctgatggcagtggttacaggggtcaattcagaggttcagctgcagcagtctggggctgagcttgtgaggccaggggcctcagtcaagttgtcctgcaaagcttctggcttcaacattaaagactactatatgcacggggtgaaacataggcctgaacagggcctggagtggattggaaggattgatcctgaaaatggtaatactatatatgacccgaagttccagggcaaggccactataacagcagacacatcctccaacacagcctacctgcagctcagcagcctgacatctgaggacactgccgtctattactgtgctagatccccttatgattaccacgcctggtttgcttactggggccaagggactctggtcaccgtctcg

SEQ ID NO: 32 - protein, 128 aa, Mus musculus
MMSSAQFLGILLLWFPGIKCDIKMTQSPSSMYTSLGERVTITCKASQDISNYFTWFQQKPGKSPKTLIYRANRLVDGVPSRFSGSGSGQDFSLTISSLEYEDMGIYYCLQYDEFPYTFGGGTKLELKR

SEQ ID NO: 33 - DNA, 381 bp, Mus musculus
atgatgtcctctgctcagtttcttggaatcttgttgctctggtttccaggtatcaaatgtgacatcaagatgacccagtctccatcttccatgtatacatctctaggcgagagagtcactatcacttgcaaggcgagtcaggacattagtaactattttacctggttccagcagaaaccagggaaatctcctaagaccctgatctatcgtgcaaacagattggtagatggggtcccatcaaggttcagtggcagtggatctgggcaagatttttctctcaccatcagcagcctcgaatatgaagatatgggaatttattattgtctacagtatgatgagtttccgtacacgttcggaggggggaccaagttggagctcaaa

SEQ ID NO: 34 - protein, 136 aa, Mus musculus
MNFGLSLIFLVLVLKGVLCEVKLVESGGGVMQPGGSLKLSCAASGFTFRTYTMSWVRQTPEKRLEWVAYISDGGGSSYFPDTVKGRFTVSRDNAKNTLYLQMSSLRSEDTAMYYCTRHSNWYFDVWGAGTSVTVSS

SEQ ID NO: 35 - DNA, 405 bp, Mus musculus
atgaacttcgggctcagcctgattttccttgtccttgttttaaaaggtgtcctgtgtgaagtgaagttggtggagtctgggggaggtgtaatgcagcctggagggtccctgaaactctcctgtgcagcctctgggttcactttcagaacctataccatgtcttgggttcgccagactccagagaagaggctggagtgggtcgcatatattagtgatggtggtggtagttcctactttccagacactgtcaagggccgattcaccgtctccagggacaatgccaagaacaccctatacctgcaaatgagcagtctgaggtctgaggacacggccatgtattactgtacaagacattctaactggtacttcgatgtctggggcgcagggacctcagtcaccgtctcg

SEQ ID NO: 36 - protein, 128 aa, Mus musculus
MMSSAQFLGLLLLCFQGTRCDIQMTQTTSSLSASLGDRVTIRCRASQDISNYLNWYQQKPDGPVKLLIYYTSTLTSGVPSRFSGSGSGTDYSLTISNLDQEDIATYFCQQGKTFPFTFGSGTKLEIKR

SEQ ID NO: 37 - DNA, 381 bp, Mus musculus
atgatgtcctctgctcagttccttggtctcctgttgctctgttttcaaggtaccagatgtgatatccagatgacacagactacatcctccctgtctgcctctctgggagacagagtcaccatccgttgcagggcaagtcaggacattagcaattatttaaactggtatcagcagaaaccagatggacctgttaaactcctgatctactacacatctacattgacctcaggagtcccatcaaggttcagtggcagtgggtctggaacagattattctctcaccattagcaacctggaccaagaagatattgccacttacttttgccaacagggtaagacgtttccattcacgttcggctcggggacaaagttggaaataaaa

SEQ ID NO: 38 - protein, 139 aa, Mus musculus
MEWSGVFIFLLSVTAGVHSQVQLQQSGAELVRPGTSVKVSCKASGYAFTNYFIEWVKQRPGQGLEWIGAINPGSGGTNYNERFKGKATLSADKSSSTAYMQLSSLTSDDSAVYFCAREDYGDVYAMDYWGQGTSVTVSS

SEQ ID NO: 39 - DNA, 414 bp, Mus musculus
atggaatggagcggggtctttatctttctcctgtcagtaactgcaggtgttcactcccaggtccagctgcagcagtctggagctgagctggtcaggcctgggacttcagtgaaggtgtcctgcaaggcttctggatacgccttcactaattacttcatagagtgggtaaaacagaggcctggacagggccttgagtggattggagcgattaatcctggaagtggtggtactaactacaatgagaggttcaagggcaaggcaactctgtctgcagacaaatcctccagcactgcctacatgcagctcagcagcctgacatctgatgactctgcggtctatttctgtgcaagagaggactatggtgatgtctatgctatggactactggggtcaaggaacctcggtcacagtctcg

SEQ ID NO: 40 - protein, 131 aa, Mus musculus
MGIKMESHSQVFVYMLLWLSGVEGDIVMTQSHKFMSTSVGDRVSITCKASQDVDTSVAWYQQKPGQAPKLLIYWASTRHTGVPDRFTGSGSGTDFTLTISNVQSEDLADYFCQQYSSYPTFGAGTKLELKR

SEQ ID NO: 41 - DNA, 390 bp, Mus musculus
atgggcatcaagatggagtcacattctcaggtctttgtatacatgttgctgtggttgtctggtgttgaaggagacattgtgatgacccagtctcacaaattcatgtccacatcagtaggagacagggtcagcatcacctgcaaggccagtcaggatgtggatacttctgtagcctggtatcaacagaaaccagggcaagctcctaaactactgatttactgggcatccacccggcacactggagtccctgatcgcttcacaggcagtggatctgggacagatttcactctcaccattagcaatgtgcagtctgaagacttggcagattatttctgtcagcaatatagcagctatcccacgttcggtgctgggaccaagctggaactgaaa

SEQ ID NO: 42 - protein, 139 aa, Mus musculus
MEWSGVIFFLMAVVTGVNSEVQLQQSGAELVRPGASVKLSCKASGFNIKDYYMHGVKHRPEQGLEWIGRIDPENGNTIYDPKFQGKATITADTSSNTAYLQLSSLTSEDTAVYYCARSPYDYHAWFAYWGQGTLVTVSS

SEQ ID NO: 43 - DNA, 414 bp, Mus musculus
atggaatggagcggagtcatcttcttcctgatggcagtggttacaggggtcaattcagaggttcagctgcagcagtctggggctgagcttgtgaggccaggggcctcagtcaagttgtcctgcaaagcttctggcttcaacattaaagactactatatgcacggggtgaaacataggcctgaacagggcctggagtggattggaaggattgatcctgagaatggtaatactatatatgacccgaagttccagggcaaggccactataacagcagacacatcctccaacacagcctacctgcagctcagcagcctgacatctgaggacactgccgtctattactgtgctagatccccttatgattaccacgcctggtttgcttactggggccaagggactctggtcactgtctcg

SEQ ID NO: 44 - protein, 131 aa, Mus musculus
MGIKMESHSQVFVYMLLWLSGVEGDIQMTQSPSSLSASVGDRVTITCKASQDVDTSVAWYQQKPGKAPKLLIYWASTRHTGVPDRFSGSGSGTDFTLTISSLQPEDFATYYCQQYSSYPTFGQGTKLEIKR

SEQ ID NO: 45 - DNA, 393 bp, Mus musculus
atgggcatcaagatggagtcacattctcaggtctttgtatacatgttgctgtggttgtctggtgttgaaggagatatccagatgacccagtctccatcctccctgtctgcatctgtaggcgaccgtgtcaccatcacttgcaaggcaagtcaggacgttgatacctctgtagcttggtatcagcagaaaccagggaaagcccctaagctcctgatctattgggcatccactcgtcatactggggtcccagaccgcttcagtggcagtggttctgggacagatttcactctcaccatcagcagtctgcaacctgaagattttgcaacttattactgtcagcaatatagcagttaccctacgttcggtcaggggaccaagctggaaattaaacgt

SEQ ID NO: 46 - protein, 139 aa, Mus musculus
MEWSGVIFFLMAVVTGVNSEVQLVQSGAEVKKPGATVKISCKASGFNIKDYYMHGVQHAPGKGLEWIGRIDPENGNTIYDPKFQGRVTITADTSTNTAYMELSSLRSEDTAVYYCARSPYDYHAWFAYWGQGTLVTVSS

SEQ ID NO: 47 - DNA, 417 bp, Mus musculus
atggaatggagcggagtcatcttcttcctgatggcagtggttacaggggtcaattcagaggtccagctggtacagtctggggctgaggtgaagaagcctggggctacagtgaaaatctcctgcaaggcttctggattcaacatcaaagactactacatgcacggggtgcaacatgcccctggaaaagggcttgagtggattggacgtattgatcctgaaaatggtaacacaatctacgacccgaagttccagggccgcgtcaccattaccgcggacacgtctacaaacacagcctacatggagctgagcagcctgcgttctgaggacacggccgtgtattactgtgcacgttctccgtatgattatcacgcctggtttgcttactggggccaagggactctggtcaccgtctcgagc

SEQ ID NO: 48 - protein, 131 aa, Mus musculus
MKLPVLLWVLLLWVPGSTGDIVLTQSPASLAVSLGQRATIYCKASQSVDYDGDSYMNWYQQKPGQPPKVLIFAASNLESGIPARFSGSRSGTDFTLNIHPVEEEDAATYYCQQTNEDPPTFGGGTKLELKR

SEQ ID NO: 49 - DNA, 390 bp, Mus musculus
atgaagctgcctgttctgctatgggtgctgctgctctgggttccaggctccactggtgacattgtgctgacccaatctccagcttctttggctgtgtctctagggcagagggccaccatttactgcaaggccagccaaagtgttgattatgatggtgatagttatatgaactggtaccaacagaaaccaggacagccacccaaagtcctcatctttgctgcatccaatctagaatctgggatcccggccaggtttagtggcagtagatctgggacagacttcaccctcaacatccatcctgtggaggaggaggatgctgcaacctattactgtcaacaaactaatgaggatcctccgacgttcggtggcggcaccaagctggaactgaaa

SEQ ID NO: 50 - protein, 138 aa, Mus musculus
MEWSWIILFLVATATDVHSQVQLQQSGAELVKPGASVKLSCKASGYAFISFWIHWVKQRPGQGLEWIGEINPSNGRTDYNAKFKTKATLTVDRSSSTAYMQLSSLTSEDSADYYCARGGTGTWYFDVWGAGTTVTVSS

SEQ ID NO: 51 - DNA, 411 bp, Mus musculus
atggaatggagctggatcatcctctttttggtagcaacagctacagatgtccactcccaggtccaactgcagcagtctggggctgaactggtgaagcctggggcttcagtgaagctgtcctgcaaggcttctggctacgcctttattagcttctggatacactgggtgaagcagaggcctggtcaaggccttgagtggattggagagattaatcctagcaacggtcgtactgactacaatgcgaagttcaagaccaaggccacactgactgttgacagatcctcctccacagcctacatgcaactcagcagcctgacatctgaggactctgcggactattactgtgcaagaggaggaactgggacctggtacttcgatgtctggggcgcagggaccacagtcaccgtctcg

SEQ ID NO: 52 - protein, 133 aa, Mus musculus
MMSPAQFLFLLVLWIRETNGDVVMTQTPLTLSVTIGQPASISCKSSQSLLDSDGETYLNWLLQRPGQSPKRLIYLVSKLDSGVPDRFTGSGSGTDFTLKISRVETEDLGVYYCWQGTHFPYTFGGGTKLELKR

SEQ ID NO: 53 - DNA, 396 bp, Mus musculus
atgatgagtcctgcccagttcctgtttctgttagtgctctggattcgggaaaccaacggtgatgttgtgatgacccagactccactcactttgtcggttaccattggacaaccagcctccatctcttgcaagtcaagtcagagcctcttagatagtgatggagagacatatttgaattggttgttacagaggccaggccagtctccaaagcgcctaatctatctggtgtctaaactggactctggagtccctgacaggttcactggcagtggatcagggacagatttcacactgaaaatcagcagagtggagactgaggatttgggagtttattattgctggcaaggtacacattttccgtacacgttcggaggggggaccaagctggaactgaaa

SEQ ID NO: 54 - protein, 135 aa, Mus musculus
MNLGLNCVFIVFLLKGVQSEVKLEESGGGLVQPGGSMKLSCVASGFTFSNYWMNWVRQSPEKGLEWVAEIRLKSDNYATHFAESVKGRFTISRDDSKSSVYLQMNNLRAEDTGIYYCTGILFGYWGQGTLVTVSS

SEQ ID NO: 55 - DNA, 402 bp, Mus musculus
atgaacttgggactgaactgtgtattcatagtttttctcttaaaaggtgtccagagtgaagtgaaacttgaggagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctgataattatgcaacacattttgcggagtctgtgaaagggaggttcaccatctcaagagatgattccaaaagtagtgtctacctgcaaatgaacaacttaagagctgaagacactggcatttattactgtacgggtatcctctttggttactggggccaagggactctggtcactgtctcg

SEQ ID NO: 56 - protein, 132 aa, Mus musculus
MESDTILLWVLLLWVPGSTGDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPWTFGGGTKLELKR

SEQ ID NO: 57 - DNA, 393 bp, Mus musculus
atggagtcagacacaatcctgctatgggtgctgctgctctgggttccaggctccactggtgacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgctacctattactgtcagcaaagtaatgaggatccgtggacgttcggtggaggcaccaagctggagctgaaa

SEQ ID NO: 58 - protein, 136 aa, Mus musculus
MEWSWIILFLVATATDVHSQVQLLQPGAELVKPGASVKLSCKASGYTFTSYWMHWVKQRPGQGLEWIGEINPSNGRTDYNENFKSKATLTVDKSSSTAYMQLSSLTSEDSAVYYCARGGVYAMDYWGQGTSVTVSS

SEQ ID NO: 59 - DNA, 405 bp, Mus musculus
atggaatggagctggatcatcctctttttggtagcaacagctacagatgtccactcccaggtccaactgctgcagcctggggctgaactggtgaagcctggggcttcagtgaagctgtcctgcaaggcttctggctacaccttcaccagctactggatgcactgggtgaagcagaggcctgggcaaggccttgagtggattggagagattaatcctagcaacggtcgtactgactacaatgagaacttcaagagcaaggccacactgactgtagacaaatcctccagcacagcctacatgcaactcagcagcctgacatctgaggactctgcggtctattactgtgcaagagggggggtctatgctatggactactggggtcaaggaacctcagtcaccgtctcg

SEQ ID NO: 60 - protein, 132 aa, Mus musculus; identical to SEQ ID NO: 12

SEQ ID NO: 61 - DNA, 393 bp, Mus musculus; identical to SEQ ID NO: 13

SEQ ID NO: 62 - protein, 135 aa, Mus musculus
MKCSWVIFFLMAVVIGITSEIQLQQSGTELVRSGASVKLSCTASGFNIKDYYIHWVRQRPDQGLEWIGWIDPENGDTEYAPKFQDRATLTADTSSNTAYLQLTLLTSEDTAVYYCNEGSGLIPYWGQGTPVTVSS

SEQ ID NO: 63 - DNA, 402 bp, Mus musculus
atgaaatgcagctgggtcatcttcttcctgatggcagtggttataggaatcacttcagagattcagctgcagcagtctgggacagagcttgtgaggtcaggggcctcagtcaaattgtcctgcacagcttctggcttcaacattaaagactactatattcactgggtgagacagaggcctgaccagggcctggagtggattggatggattgatcctgagaatggtgatactgaatatgccccgaagttccaggacagggccactttgactgcagacacatcctccaacacagcctacctgcagctcacactcctgacatctgaggacactgccgtctattactgtaatgagggctcgggcttgattccttactggggccaagggactccggtcaccgtctcg

SEQ ID NO: 64 - protein, 128 aa, Mus musculus; identical to SEQ ID NO: 36

SEQ ID NO: 65 - DNA, 381 bp, Mus musculus; identical to SEQ ID NO: 37

SEQ ID NO: 66 - protein, 139 aa, Mus musculus
MEWSWVFIFLLSVTAGVHSQVQLQQSGAELVRPGTSVKVSCKASGYVFINYFIEWVKQRPGQGLEWIGVINPENGGTNYNERFKDKAALTADTSSSTAYMQLSSLTSDDSAVYFCAREDYGDVYAMDYWGQGTSVTVSS

SEQ ID NO: 67 - DNA, 414 bp, Mus musculus
atggaatggagctgggtctttatctttctcctgtcagttactgcaggtgttcactcccaggtccagctgcagcagtctggagctgaactggtcaggcctgggacttcagtgaaggtgtcctgcaaggcttctggatacgtcttcattaattacttcatagagtgggttaaacagaggcctggacagggccttgagtggattggagtgattaatcctgaaaatggtggtactaactacaatgagagattcaaggacaaggcagcactgactgcagacacatcctccagcactgcctacatgcaactcagcagcctgacatctgatgactctgcggtctatttctgtgcaagagaggactatggtgatgtctatgctatggactactggggtcaaggaacctcagtcacagtctcg

SEQ ID NO: 68 - protein, 132 aa, Mus musculus; identical to SEQ ID NO: 12

SEQ ID NO: 69 - DNA, 393 bp, Mus musculus; identical to SEQ ID NO: 13

SEQ ID NO: 70 - protein, 135 aa, Mus musculus
MKCSWVIFFLMAVVIGINSEVQLQQSGTELVRSGASVRLSCTASGFNIKDYYIHWVKQRPEQGLEWIGWIDPENGDSEYAPKFQDKATMTADTSSNTAYLQLSSLTSEDTAVYYCNEGSGLIPYWGHGTLVTVSS

SEQ ID NO: 71 - DNA, 402 bp, Mus musculus
atgaaatgcagctgggtcatcttcttcctgatggcagtggttataggaatcaattcagaggttcagctgcagcagtctgggacagagcttgtgaggtcaggggcctcagtcaggttgtcctgcacagcttctggcttcaacattaaagactactatatacactgggtgaaacagaggcctgaacagggcctggagtggattggatggattgatcctgagaatggtgattctgaatatgccccgaagttccaggacaaggccaccatgactgcagacacatcctccaacacagcctacctgcagctcagcagcctgacatctgaggacactgccgtctattattgtaatgagggctcgggcttgattccttactggggccacgggactctggtcacagtctcg

SEQ ID NO: 72 - protein, 133 aa, Mus musculus
MSVPTQVLGLLLLWLTDARCDIVLTQTPLSLPVSLGDHASISCRSSQSLVHSNGDVYFHWYLQKPGQSPKLLIYEVSNRFSGVPDRFSGSGSGTYFTLKISRVEAEDLGVYFCSQSTHVPYTFGGGTKLELKR

SEQ ID NO: 73 - DNA, 405 bp, Mus musculus
atgtctgtccccacccaagtcctcggactcctgctgctgtggcttacagatgccagatgcgaggttcagctgcagcagtctgggacagagcttgtgaggtcaggggcctcagtcaggttgtcctgcacagcttctggcttcaacattaaagactactatatacactgggtgaaacagaggcctgaacagggcctggagtggattggatggattgatcctgagaatggtgattctgaatatgccccgaagttccaggacaaggccaccatgactgcagacacatcctccaacacagcctacctgcagctcagcagcctgacatctgaggacactgccgtctattattgtaatgagggctcgggcttgattccttactggggccacgggactctggtcacagtctcg

SEQ ID NO: 74 - protein, 135 aa, Mus musculus; identical to SEQ ID NO: 70

SEQ ID NO: 75 - DNA, 393 bp, Mus musculus
atgaaatgcagctgggtcatcttcttcctgatggcagtggttataggaatcaattcagatattgtgttgacccaaactcctctctctctgcctgtcagtcttggagatcatgcctccatctcttgcagatctagtcagagccttgtacacagtaatggcgacgtctatttccattggtacctgcagaagccaggccagtctccaaagctcctgatctacgaagtttccaaccgattttctggggtcccagacaggttcagtggcagtggatcagggacatatttcacactcaagatcagcagagtggaggctgaggatctgggagtttatttctgctctcaaagtacacatgttccgtacactttcggaggggggaccaagttggagctcaaa

SEQ ID NO: 76 - protein, 133 aa, Mus musculus; identical to SEQ ID NO: 72

SEQ ID NO: 77 - DNA, 396 bp, Mus musculus
atgtctgtccccacccaagtcctcggactcctgctgctgtggcttacagatgccagatgcgatgttgtgttgacccaaactccactctccctgcctgtcagtcttggagatcaagcctccatctcttgcagatctagtcagagccttgtacacagtaatggagacgtctatttacattggtacctgcagaagccaggccagtctccaaagctcctgatctacgaagtttccaaccgattttctggggtcccagacaggttcagtggcagtggatcagggacagatttcacactcaagatcaacagagtggaggctgaggatctgggagtttatttctgctctcaaagtacacatgttccgtacacgttcggaggggggaccaagctggaactgaaa

SEQ ID NO: 78 - protein, 135 aa, Mus musculus
MEWSWVIFFLMAVVIGINSEVQLQQSGTELVRSGASVKLSCTASGFNIKDYYVHWVKQRPEQGLEWIGWIDPDNGDSEYAPKFQDKATMTADTSSNTAYLQLNSLTSEDTAVYYCNEGSGLIPYWGPGTLVTVSS

SEQ ID NO: 79 - DNA, 402 bp, Mus musculus
atggaatggagctgggtcatcttcttcctgatggcagtggttataggaatcaattctgaggttcagctgcagcagtctgggacagagcttgtgaggtcaggggcctcagtcaagttgtcctgcacagcttctggcttcaacattaaagactactatgtacactgggtgaagcagaggcctgaacagggcctggaatggattggatggattgatcctgacaatggtgatagtgaatatgccccgaagttccaggacaaggccactatgactgcagacacatcctccaacacagcctaccttcaactcaacagcctgacttctgaggacactgccgtctattattgtaatgagggctcgggcttgattccttactggggcccagggactctggtcacagtctcg

SEQ ID NO: 80 - protein, 133 aa, Mus musculus
MSVPTQVLGLLLLWLTDARCDVVMTQTPLSLPVSLGDQASISCRSSQSLVHSNGDVYLHWYLQKPGQSPKLLIYEVSNRFSGVPDRFSGSGSGTDFTLKISRVEAEDLGVYFCSQTTHVPYTFGGGTKLELKR

SEQ ID NO: 81 - DNA, 405 bp, Mus musculus
atgtctgtccccacccaagtcctcggactcctgctgctgtggcttacagatgccagatgcgagattcagctgcagcagtctgggacagagcttgtgaggtcaggggcctcagtcaaattgtcctgcacagcttctggcttcaacattaaagactactatattcactgggtgagacagaggcctgaccagggcctggagtggattggatggattgatcctgagaatggtgatactgaatatgccccgaagttccaggacagggccactttgactgcagacacatcctccaacacagcctacctgcagctcacactcctgacatctgaggacactgccgtctattactgtaatgagggctcgggcttgattccttactggggccaagggactccggtcaccgtctcg

SEQ ID NO: 82 - protein, 135 aa, Mus musculus; identical to SEQ ID NO: 62

SEQ ID NO: 83 - DNA, 393 bp, Mus musculus
atgaaatgcagctgggtcatcttcttcctgatggcagtggttataggaatcacttcagatgttgtgatgacccaaactccactctccctgcctgtcagtcttggagatcaagcctccatctcctgcagatctagtcagagccttgtacacagtaatggagacgtctatttacattggtacctgcagaagccaggccagtctccaaaactcctgatctacgaagtttccaaccgattttctggggtcccagacaggttcagtggcagtggttcagggacagatttcacactcaagatcagcagagtggaggctgaggatctgggagtttatttctgttctcaaactacacatgttccgtacacgttcggaggggggaccaagctggagctgaaa

SEQ ID NO: 84 - protein, 133 aa, Mus musculus
MSVPTQVLGLLLLWLTDARCDVVMTQTPLSLPVSLGDQASISCRSSQSLVHSNGDVYLHWYLQKPGQSPKLLIYEVSNRFSGVPDRFTGSGSGTDFTLKISRVEAEDLGVFFCSQSTHVPYTFGGGTKLELKR

SEQ ID NO: 85 - DNA, 396 bp, Mus musculus
atgtctgtccccacccaagtcctcggactcctgctgctgtggcttacagatgccagatgcgatgttgtgatgacccaaactccactctccctgcctgtcagtcttggagatcaagcctccatctcctgcagatctagtcagagccttgtacacagtaatggagacgtctatttacattggtacctgcagaagccaggccagtctccaaaactcctgatctacgaagtttccaaccgattttctggggtcccagacaggttcactggcagtggttcagggacagatttcacactcaagatcagcagagtggaggctgaggatctgggagtttttttctgctctcaaagtacacatgttccgtacacgttcggaggggggaccaagctggaactgaaa

SEQ ID NO: 86 - protein, 135 aa, Mus musculus
MKCSWVIFFLMAVVIGITSEIQLQQSGTELVRSGASVKLSCTASGFNIKDYYIHWVKQRPDQGLEWIGWIDPENGDTEYAPKFQDRATMTADTSSNTAYLQLTILTSEDTAVYYCNEGSGLIPYWGQGTLVTVSS

SEQ ID NO: 87 - DNA, 402 bp, Mus musculus
atgaaatgcagctgggtcatcttcttcctgatggcagtggttataggaatcacttcagagattcagctgcagcagtctgggacagagcttgtgaggtcaggggcctcagtcaaattgtcctgcacagcttctggcttcaacattaaagactactatattcactgggtgaaacagaggcctgaccagggcctggagtggattggatggattgatcctgagaatggtgatactgaatatgccccgaagttccaggacagggccactatgactgcagacacatcctccaacacagcctacctgcagctcaccatcctgacatctgaggacactgccgtctattactgtaatgagggctcgggcttgattccttactggggccaagggactctggtcaccgtctcg

SEQ ID NO: 88 - protein, 128 aa, Mus musculus
MSVPTQVLGLLLLWLTDARCDIQMNQSPSSLSASLGDTITITCHASQNINVWLSWYQQKPGNIPKLLIYKVSNLHTGVPSRFSGSGSGTGFTLTISSLQPEDIATYYCQQGQSYPLTFGGGTKLELKR

SEQ ID NO: 89 - DNA, 381 bp, Mus musculus
atgtctgtccccacccaagtcctcggactcctgctgctgtggcttacagatgccagatgcgacatccagatgaaccagtctccatccagtctgtctgcatcccttggagacacaattaccatcacttgccatgccagtcagaacattaatgtttggttaagctggtaccagcagaaaccaggaaatattcctaaactattgatctataaggtttccaacttgcacacaggcgtcccatcaaggtttagtggcagtggatctggaacaggtttcacattaaccatcagcagcctgcagcctgaagacattgccacttactactgtcaacagggtcaaagttatcctctgacgttcggtggaggcaccaagttggagctcaaa

SEQ ID NO: 90 - protein, 139 aa, Mus musculus
MGWNWIIFFLMAVVTGVNSEVQLQQSGAELVRPGALVKLSCKASGFNIKDYYIHWVKQRPEQGLEWIGRIDPENGNTIYDPKFQGKAIITADTSSNTAYLQLSSLTSEDTAVYYCARCDNDPGSEMDYWGQGTTVTVSS

SEQ ID NO: 91 - DNA, 414 bp, Mus musculus
atgggatggaactggatcatcttcttcctgatggcagtggttacaggggtcaattcagaggttcagctgcagcagtctggggctgagcttgtgaggccaggggccttagtcaaattgtcctgcaaagcttctggattcaacattaaagactactatatacactgggtgaagcagaggcctgaacagggcctggagtggattggaaggattgatcctgaaaatggtaatactatatatgacccgaagttccagggcaaggccattataacagcagacacatcctccaacacagcctacctgcagctcagcagcctgacatctgaggacactgccgtctattactgtgctagatgtgataacgaccccggctctgaaatggactactggggtcaaggaaccacggtcaccgtctcg

SEQ ID NO: 92 - protein, 133 aa, Mus musculus; identical to SEQ ID NO: 72

SEQ ID NO: 93 - DNA, 396 bp, Mus musculus
atgtctgtccccacccaagtcctcggactcctgctgctgtggcttacagatgccagatgcgatattgtgttgacccaaactcctctctctctgcctgtcagtcttggagatcatgcctccatctcttgcagatctagtcagagccttgtacacagtaatggcgacgtctatttccattggtacctgcagaagccaggccagtctccaaagctcctgatctacgaagtttccaaccgattttctggggtcccagacaggttcagtggcagtggatcagggacatatttcacactcaagatcagcagagtggaggctgaggatctgggagtttatttctgctctcaaagtacacatgttccgtacactttcggaggggggaccaagttggagctcaaa

SEQ ID NO: 94 - protein, 135 aa, Mus musculus; identical to SEQ ID NO: 78

SEQ ID NO: 95 - DNA, 402 bp, Mus musculus; identical to SEQ ID NO: 79

SEQ ID NO: 96 - protein, 106 aa, Mus musculus
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 327 aa, Mus musculus
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 98 - protein, 15 aa, Mus musculus
KASQSVDYAGDNYMN

SEQ ID NO: 99 - protein, 7 aa, Mus musculus
TASNLES

SEQ ID NO: 100 - protein, 9 aa, Mus musculus
QQSNEDPPT

SEQ ID NO: 101 - protein, 5 aa, Mus musculus
GYFMH

SEQ ID NO: 102 - protein, 17 aa, Mus musculus
EINPSTGGTTYNQRFKG

SEQ ID NO: 103 - protein, 10 aa, Mus musculus
WGYNPYALDY

SEQ ID NO: 104 - protein, 11 aa, Mus musculus
KASQDINKYIA

SEQ ID NO: 105 - protein, 7 aa, Mus musculus
YTSTLQP

SEQ ID NO: 106 - protein, 8 aa, Mus musculus
LQYDNLYT

SEQ ID NO: 107 - protein, 5 aa, Mus musculus
SYWIE

SEQ ID NO: 108 - protein, 17 aa, Mus musculus
EIFPRNGSTYYNEKFKG

SEQ ID NO: 109 - protein, 6 aa, Mus musculus
INTLDY

SEQ ID NO: 110 - protein, 16 aa, Mus musculus
RSSQSLVHSNGDTYLH

SEQ ID NO: 111 - protein, 7 aa, Mus musculus
EISNRFS

SEQ ID NO: 112 - protein, 9 aa, Mus musculus
SQSTHVPFT

SEQ ID NO: 113 - protein, 5 aa, Mus musculus
DYYMH

SEQ ID NO: 114 - protein, 17 aa, Mus musculus
WNDPETGDTEYAPKFQG

SEQ ID NO: 115 - protein, 7 aa, Mus musculus
GSGLIPY

SEQ ID NO: 116 - protein, 16 aa, Mus musculus
RSSQSLVHSNGDTYLH

SEQ ID NO: 117 - protein, 7 aa, Mus musculus
EISNRFS

SEQ ID NO: 118 - protein, 9 aa, Mus musculus
SQSTHVPFT

SEQ ID NO: 119 - protein, 5 aa, Mus musculus
DYYMH

SEQ ID NO: 120 - protein, 17 aa, Mus musculus
WNDPETGDTEYAPKFQG

SEQ ID NO: 121 - protein, 7 aa, Mus musculus
GSGLIPY

SEQ ID NO: 122 - protein, 16 aa, Mus musculus
RSSQSLVHSNGDVYLH

SEQ ID NO: 123 - protein, 7 aa, Mus musculus
EVSNRFS

SEQ ID NO: 124 - protein, 9 aa, Mus musculus
SQSTHVPFT

SEQ ID NO: 125 - protein, 5 aa, Mus musculus
NYYMH

SEQ ID NO: 126 - protein, 17 aa, Mus musculus
WIDPENGDTEYAPKFQD

SEQ ID NO: 127 - protein, 7 aa, Mus musculus
GSGLIPY

SEQ ID NO: 128 - protein, 16 aa, Mus musculus
RSSQSLVHSNGNTYLY

SEQ ID NO: 129 - protein, 7 aa, Mus musculus
KVSTRFS

SEQ ID NO: 130 - protein, 9 aa, Mus musculus
SQSSHIPPT

SEQ ID NO: 131 - protein, 5 aa, Mus musculus
DYGMN

SEQ ID NO: 132 - protein, 17 aa, Mus musculus
WIDTYTEKPTYADDFKG

SEQ ID NO: 133 - protein, 5 aa, Mus musculus
SNFDF

SEQ ID NO: 134 - protein, 11 aa, Mus musculus
KASQDVDTSVA

SEQ ID NO: 135 - protein, 7 aa, Mus musculus
WASTRHT

SEQ ID NO: 136 - protein, 8 aa, Mus musculus
QQYSNYPT

SEQ ID NO: 137 - protein, 5 aa, Mus musculus
DYYMH

SEQ ID NO: 138 - protein, 17 aa, Mus musculus
RIDPENGNTIYDPKFQG

SEQ ID NO: 139 - protein, 11 aa, Mus musculus
SPYDYHAWFAY

SEQ ID NO: 140 - protein, 11 aa, Mus musculus
KASQDISNYFT

SEQ ID NO: 141 - protein, 7 aa, Mus musculus
RANRLVD

SEQ ID NO: 142 - protein, 9 aa, Mus musculus
LQYDEFPYT

SEQ ID NO: 143 - protein, 5 aa, Mus musculus
TYTMS

SEQ ID NO: 144 - protein, 17 aa, Mus musculus
YISDGGGSSYFPDTVKG

SEQ ID NO: 145 - protein, 8 aa, Mus musculus
HSNWYFDV

SEQ ID NO: 146 - protein, 11 aa, Mus musculus
RASQDISNYLN

SEQ ID NO: 147 - protein, 7 aa, Mus musculus
YTSTLTS

SEQ ID NO: 148 - protein, 9 aa, Mus musculus
QQGKTFPFT

SEQ ID NO: 149 - protein, 5 aa, Mus musculus
NYFIE

SEQ ID NO: 150 - protein, 17 aa, Mus musculus
AINPGSGGTNYNERFKG

SEQ ID NO: 151 - protein, 11 aa, Mus musculus
EDYGDVYAMDY

SEQ ID NO: 152 - protein, 11 aa, Mus musculus
KASQDVDTSVA

SEQ ID NO: 153 - protein, 7 aa, Mus musculus
WASTRHT

SEQ ID NO: 154 - protein, 8 aa, Mus musculus
QQYSSYPT

SEQ ID NO: 155 - protein, 5 aa, Mus musculus
DYYMH

SEQ ID NO: 156 - protein, 17 aa, Mus musculus
RIDPENGNTIYDPKFQG

SEQ ID NO: 157 - protein, 11 aa, Mus musculus
SPYDYHAWFAY

SEQ ID NO: 158 - protein, 11 aa, Mus musculus
KASQDVDTSVA

SEQ ID NO: 159 - protein, 7 aa, Mus musculus
WASTRHT

SEQ ID NO: 160 - protein, 8 aa, Mus musculus
QQYSSYPT

SEQ ID NO: 161 - protein, 5 aa, Mus musculus
DYYMH

SEQ ID NO: 162 - protein, 17 aa, Mus musculus
RIDPENGNTIYDPKFQG

SEQ ID NO: 163 - protein, 11 aa, Mus musculus
SPYDYHAWFAY

SEQ ID NO: 164 - protein, 15 aa, Mus musculus
KASQSVDYDGDSYMN

SEQ ID NO: 165 - protein, 7 aa, Mus musculus
AASNLES

SEQ ID NO: 166 - protein, 9 aa, Mus musculus
QQTNEDPPT

SEQ ID NO: 167 - protein, 5 aa, Mus musculus
SFWIH

SEQ ID NO: 168 - protein, 17 aa, Mus musculus
EINPSNGRTDYNAKFKT

SEQ ID NO: 169 - protein, 10 aa, Mus musculus
GGTGTWYFDV

SEQ ID NO: 170 - protein, 16 aa, Mus musculus
KSSQSLLDSDGETYLN

SEQ ID NO: 171 - protein, 7 aa, Mus musculus
LVSKLDS

SEQ ID NO: 172 - protein, 9 aa, Mus musculus
WQGTHFPYT

SEQ ID NO: 173 - protein, 5 aa, Mus musculus
NYWMN

SEQ ID NO: 174 - protein, 19 aa, Mus musculus
EIRLKSDNYATHFAESVKG

SEQ ID NO: 175 - protein, 5 aa, Mus musculus
ILFGY

SEQ ID NO: 176 - protein, 15 aa, Mus musculus
KASQSVDYDGDSYMN

SEQ ID NO: 177 - protein, 7 aa, Mus musculus
AASNLES

SEQ ID NO: 178 - protein, 9 aa, Mus musculus
QQSNEDPWT

SEQ ID NO: 179 - protein, 5 aa, Mus musculus
SYWMH

SEQ ID NO: 180 - protein, 17 aa, Mus musculus
EINPSNGRTDYNENFKS

SEQ ID NO: 181 - protein, 8 aa, Mus musculus
GGVYAMDY

SEQ ID NO: 182 - protein, 16 aa, Mus musculus
RSSQSLVHSNGDTYLH

SEQ ID NO: 183 - protein, 7 aa, Mus musculus
EISNRFS

SEQ ID NO: 184 - protein, 9 aa, Mus musculus
SQSTHVPFT

SEQ ID NO: 185 - protein, 5 aa, Mus musculus
DYYIH

SEQ ID NO: 186 - protein, 17 aa, Mus musculus
WIDPENGDTEYAPKFQD

SEQ ID NO: 187 - protein, 7 aa, Mus musculus
GSGLIPY

SEQ ID NO: 188 - protein, 11 aa, Mus musculus
RASQDISNYLN

SEQ ID NO: 189 - protein, 7 aa, Mus musculus
YTSTLTS

SEQ ID NO: 190 - protein, 9 aa, Mus musculus
QQGKTFPFT

SEQ ID NO: 191 - protein, 5 aa, Mus musculus
NYFIE

SEQ ID NO: 192 - protein, 17 aa, Mus musculus
VINPENGGTNYNERFKD

SEQ ID NO: 193 - protein, 11 aa, Mus musculus
EDYGDVYAMDY

SEQ ID NO: 194 - protein, 16 aa, Mus musculus
RSSQSLVHSNGDTYLH

SEQ ID NO: 195 - protein, 7 aa, Mus musculus
EISNRFS

SEQ ID NO: 196 - protein, 9 aa, Mus musculus
SQSTHVPFT

SEQ ID NO: 197 - protein, 5 aa, Mus musculus
DYYIH

SEQ ID NO: 198 - protein, 17 aa, Mus musculus
WIDPENGDSEYAPKFQD

SEQ ID NO: 199 - protein, 7 aa, Mus musculus
GSGLIPY

SEQ ID NO: 200 - protein, 16 aa, Mus musculus
RSSQSLVHSNGDVYFH

SEQ ID NO: 201 - protein, 7 aa, Mus musculus
EVSNRFS

SEQ ID NO: 202 - protein, 9 aa, Mus musculus
SQSTHVPYT

SEQ ID NO: 203 - protein, 5 aa, Mus musculus
DYYIH

SEQ ID NO: 204 - protein, 17 aa, Mus musculus
WIDPENGDSEYAPKFQD

SEQ ID NO: 205 - protein, 7 aa, Mus musculus
GSGLIPY

SEQ ID NO: 206 - protein, 16 aa, Mus musculus
RSSQSLVHSNGDVYLH

SEQ ID NO: 207 - protein, 7 aa, Mus musculus
EVSNRFS

SEQ ID NO: 208 - protein, 9 aa, Mus musculus
SQSTHVPYT

SEQ ID NO: 209 - protein, 5 aa, Mus musculus
DYYVH

SEQ ID NO: 210 - protein, 17 aa, Mus musculus
WIDPDNGDSEYAPKFQD

SEQ ID NO: 211 - protein, 7 aa, Mus musculus
GSGLIPY

SEQ ID NO: 212 - protein, 16 aa, Mus musculus
RSSQSLVHSNGDVYLH

SEQ ID NO: 213 - protein, 7 aa, Mus musculus
EVSNRFS

SEQ ID NO: 214 - protein, 9 aa, Mus musculus
SQTTHVPYT

SEQ ID NO: 215 - protein, 5 aa, Mus musculus
DYYIH

SEQ ID NO: 216 - protein, 17 aa, Mus musculus
WIDPENGDTEYAPKFQD

SEQ ID NO: 217 - protein, 7 aa, Mus musculus
GSGLIPY

SEQ ID NO: 218 - protein, 16 aa, Mus musculus
RSSQSLVHSNGDVYLH

SEQ ID NO: 219 - protein, 7 aa, Mus musculus
EVSNRFS

SEQ ID NO: 220 - protein, 9 aa, Mus musculus
SQSTHVPYT

SEQ ID NO: 221 - protein, 5 aa, Mus musculus
DYYIH

SEQ ID NO: 222 - protein, 17 aa, Mus musculus
WIDPENGDTEYAPKFQD

SEQ ID NO: 223 - protein, 7 aa, Mus musculus
GSGLIPY

SEQ ID NO: 224 - protein, 11 aa, Mus musculus
HASQNINVWLS

SEQ ID NO: 225 - protein, 7 aa, Mus musculus
KVSNLHT

SEQ ID NO: 226 - protein, 9 aa, Mus musculus
QQGQSYPLT

SEQ ID NO: 227 - protein, 5 aa, Mus musculus
DYYIH

SEQ ID NO: 228 - protein, 17 aa, Mus musculus
RIDPENGNTIYDPKFQG

SEQ ID NO: 229 - protein, 11 aa, Mus musculus
CDNDPGSEMDY

SEQ ID NO: 230 - protein, 16 aa, Mus musculus
RSSQSLVHSNGDVYFH

SEQ ID NO: 231 - protein, 7 aa, Mus musculus
EVSNRFS

SEQ ID NO: 232 - protein, 9 aa, Mus musculus
SQSTHVPYT

SEQ ID NO: 233 - protein, 5 aa, Mus musculus
DYYVH

SEQ ID NO: 234 - protein, 17 aa, Mus musculus
WIDPDNGDSEYAPKFQD

SEQ ID NO: 235 - protein, 7 aa, Mus musculus
GSGLIPY

SEQ ID NO: 236 - protein, 112 aa, Mus musculus
DIVLTQSPASLAVSLGQRATISCKASQSVDYAGDNYMNWYQQKPGQPPKLLIYTASNLESGIPARFSGSGSGTDFTLNIHPVEEEDAATYYCQQSNEDPPTFGGGTKLELKR

SEQ ID NO: 237 - DNA, 336 bp, Mus musculus
gacattgtgctgacccaatctccagcttctttggctgtgtctctagggcagagggccaccatctcctgcaaggccagccaaagtgttgattatgctggtgataattatatgaactggtaccaacagaaaccaggacagccacccaaactcctcatctatactgcatccaatctagagtctgggatcccagccaggtttagtggcagtgggtctgggacagacttcaccctcaacattcatcctgtggaggaggaggatgctgcaacctattactgtcagcaaagtaatgaggatcctccgacgttcggtggaggcaccaagttggagctcaaacgt

SEQ ID NO: 238 - protein, 119 aa, Mus musculus
EVQLQQSGPELVKPGASVKISCKASGYPFTGYFMHWVKQSPENSLEWIGEINPSTGGTTYNQRFKGKATLTVDKSSSTAYMQLKSLTSEDSAVYYCTRWGYNPYALDYWGQGTSVTVSS

SEQ ID NO: 239 - DNA, 357 bp, Mus musculus
gaggtccaactgcagcagtctggacctgagctggtgaagcctggggcttcagtgaagatatcctgcaaggcttctggttacccattcactggctacttcatgcactgggtgaaacaaagtcctgaaaatagtcttgagtggattggagagattaatcctagcactgggggtactacctacaaccagaggttcaagggcaaggccacattaactgtagataaatcctccagcacagcctacatgcagctcaagagcctgacatctgaagactctgcagtctattactgtacaagatggggatataacccctatgctttggactactggggtcaaggaacctcagtcaccgtctcgagc

SEQ ID NO: 240 - protein, 107 aa, Mus musculus
DIQMTQSPSSLSASLGGKVTITCKASQDINKYIAWYQHKPGKGPRLLIHYTSTLQPGIPSRFSGSGSGRDYSFSISNLEPEDIATYYCLQYDNLYTFGGGTKLELKR

SEQ ID NO: 241 - DNA, 321 bp, Mus musculus
gacatccagatgacacagtctccatcctcactgtctgcatctctgggaggcaaagtcaccatcacttgcaaggcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatctatacacgttcggaggggggaccaagctggaactgaaacgt

SEQ ID NO: 242 - protein, 115 aa, Mus musculus
QVQLQQSGAELMKPGASVKISCKATGYTFSSYWIEWVKQRPGHGPEWIGEIFPRNGSTYYNEKFKGKATFTADTSSNTAYMQLSSLTSDDSAVYYCAIINTLDYWGQGTTLTVSS

SEQ ID NO: 243 - DNA, 345 bp, Mus musculus
caggttcagctgcagcagtctggagctgagctgatgaagcctggggcctcagtgaagatatcctgcaaggctactggctacacattcagtagttattggatagagtgggtaaagcagaggcctggacatggccctgagtggattggagagatttttcctagaaatggtagtacttactacaatgagaaattcaagggcaaggccacattcactgcagatacatcctccaacacagcctacatgcaactcagcagcctgacatctgacgactctgccgtctattactgtgcaattattaatacgcttgactactggggccaaggcaccactctcacagtctcgagc

SEQ ID NO: 244 - protein, 113 aa, Mus musculus
DVVMTQTPLSLPVSLGDQASISCRSSQSLVHSNGDTYLHWYLQKPGQSPKLLIYEISNRFSGVPDRFSGSGSGTDFTLKISRVEAEDLGVYFCSQSTHVPFTFGGGTKLEIKR

SEQ ID NO: 245 - DNA, 339 bp, Mus musculus
gatgttgtgatgacccaaactccactctccctgcctgtcagtcttggagatcaagcctccatctcttgcagatctagtcagagccttgtacacagtaatggagacacctatttacattggtacctacagaagccaggccagtctccaaaactcctgatctacgaaatttccaaccgattttctggggtcccagacaggttcagtggcagtggatcagggacagatttcacactcaagatcagcagagtggaggctgaggatctgggagtttatttctgctctcaaagtacacatgttccgttcacgttcggaggggggaccaagctggaaataaaacgt

SEQ ID NO: 246 - protein, 116 aa, Mus musculus
EVQLQQSGAELVRSGASVRLSCTASGFNIKDYYMHWVRQRPEQGLEWIGWNDPETGDTEYAPKFQGKATMTSDTSSNTAYLQLSSLTSEDTAVYYCNEGSGLIPYWGPGTLVTVSS

SEQ ID NO: 247 - DNA, 348 bp, Mus musculus
gaggttcagctgcagcagtctggggcagaacttgtgaggtcaggggcctcagtcaggttgtcctgcacagcttctggcttcaacattaaagactactatatgcactgggtgaggcagcggcctgaacagggcctggagtggattggatggaatgatcctgagactggtgatactgaatatgccccgaagttccagggcaaggccactatgacttcagacacatcctccaacacagcctacctgcagctcagcagcctgacatctgaggacactgccgtctattactgtaatgagggctcgggcttgattccttactggggcccagggactctggtcacagtctcgagc

SEQ ID NO: 248 - protein, 113 aa, Mus musculus
DIVMTQTPLSLSVTPGQPASISCRSSQSLVHSNGDTYLHWYLQKPGQPPQLLIYEISNRFSGVPDRFSGSGSGTDFTLKISRVEAEDVGVYYCSQSTHVPFTFGQGTKLEIKR

SEQ ID NO: 249 - DNA, 339 bp, Mus musculus
gatatcgtgatgacccagactccactctccctgtctgtcactccgggtcaaccggcctccatctcttgccgttctagtcagagccttgtacacagtaatggtgacacctatttacattggtacttacagaagccaggccagcctccacaactcctgatctacgaaatttccaaccgcttttctggggtcccagaccgtttcagtggcagtggttcagggacagatttcacactcaagatcagccgcgtggaggctgaggatgtgggtgtttattactgctctcaaagtacacatgttccgttcacgttcggtcaggggaccaagctggaaattaaacgt

SEQ ID NO: 250 - protein, 116 aa, Mus musculus
QVQLVQSGAEVKKPGSSVKVSCKASGFNIKDYYMHWVRQAPGQGLEWMGWNDPETGDTEYAPKFQGRVTITADESTNTAYMELSSLRSEDTAVYYCNEGSGLIPYWGQGTLVTVSS

SEQ ID NO: 251 - DNA, 348 bp, Mus musculus
caggttcagctggtccagtctggggcagaggttaaaaagccagggtcctcagtcaaagtgtcctgcaaggcttctggcttcaacattaaagactactatatgcactgggtgagacaggcgcctggtcagggcctggagtggatgggatggaatgatcctgagactggtgatactgaatatgccccgaagttccagggcagggtcactattactgcagacgaatccaccaacacagcctacatggagctctcatccctgcgttctgaggacactgccgtctattactgtaatgagggctcgggcttgattccttactggggccaagggactctggtcaccgtctcgagc

SEQ ID NO: 252 - protein, 113 aa, Mus musculus
DVVLTQTPLSLPVSLGDQASISCRSSQSLVHSNGDVYLHWYLQKPGQSPKLLIYEVSNRFSGVPDRFSGSGSGTDFTLKISRVEAEDLGVYFCSQSTHVPFTFGGGTKLEIKR

SEQ ID NO: 253 - DNA, 339 bp, Mus musculus
gatgttgtattgacccaaactccactctccctgcctgtcagtcttggagatcaagcctccatctcttgtagatctagtcagagccttgtacacagtaatggagacgtctatttacattggtacctacagaagccaggccagtctccaaagctcctgatctacgaagtttccaaccgattttctggggtcccagacaggttcagtggcagtggatcagggacagatttcacactcaagatcagcagagtggaggctgaggatctgggagtttatttctgctctcaaagtacacatgttccgttcacgttcggaggggggaccaagctggaaataaaacgt

SEQ ID NO: 254 - protein, 116 aa, Mus musculus
EVQLQQSGTELVRSGASVKLACTASGFNIKNYYMHWVRQRPEQGLEWIGWIDPENGDTEYAPKFQDKATMTADTSSNTAYLQLSSLTSEDTAVYYCNEGSGLIPYWGPGTLVTVSS

SEQ ID NO: 255 - DNA, 348 bp, Mus musculus
gaggttcagctgcagcagtctgggacagagcttgtgaggtcaggggcctcagtcaagttggcctgcacagcttctggcttcaacattaaaaactactatatgcactgggtgaggcagaggcctgaacagggcctggagtggattggatggattgatcctgagaatggtgatactgagtatgccccgaagttccaggacaaggccactatgactgcagacacatcctccaacacagcctacctgcagctcagcagcctgacatctgaggacactgccgtctattactgtaatgagggctcgggcttgattccttactggggcccagggactctggtcactgtctcgagc

SEQ ID NO: 256 - protein, 113 aa, Mus musculus
GVVMTQTPLSLPVSLGDQASISCRSSQSLVHSNGNTYLYWYLQKPGQSPKLLLFKVSTRFSGVPDRFSGSGSGTDFTLKITRVEAEDLGIYFCSQSSHIPPTFGGGTKLELKR

SEQ ID NO: 257 - DNA, 339 bp, Mus musculus
ggtgttgtgatgacccaaactccactctccctgcctgtcagtcttggagatcaagcctccatctcgtgcagatctagtcagagccttgttcacagtaatggaaacacctatttatattggtacctacagaagccaggccagtctccaaagctcctgctcttcaaagtttccacccgattttctggggtcccagacaggttcagtggcagtggatcagggacagatttcacactcaagatcaccagagtagaggctgaggatctgggaatttatttctgctctcaaagttcacatattcctccgacgttcggtggaggcaccaagttggagctcaaacgt

SEQ ID NO: 258 - protein, 114 aa, Mus musculus
QIQLVQSGPELKKPGETVKISCKASGYTVTDYGMNWVKQSPGKDLKWMGWIDTYTEKPTYADDFKGRFAFSLETSASTAYLQISNLKNEDSAAYFCLRSNFDFWGQGTTLTVSS

SEQ ID NO: 259 - DNA, 342 bp, Mus musculus
cagatccagttggtgcagtctggacctgagctgaagaagcctggagagacagtcaagatctcctgcaaggcttctgggtataccgtcacagactatggaatgaactgggtgaagcagagtccaggaaaggatttaaagtggatgggctggatagacacctacactgaaaagccaacatatgctgatgacttcaagggacgatttgccttctctttggaaacctctgccagcactgcctacttgcagatcagcaacctcaaaaatgaggactcggctgcatatttctgtctaagatccaattttgacttttggggccaaggcaccactctcacagtctcgagc

SEQ ID NO: 260 - protein, 107 aa, Mus musculus
DIVMTQSHKFMSTSVGGRVSITCKASQDVDTSVAWYQQKPGQAPKLLVYWASTRHTGVPDRFTGSGSGTDFTLTISNVQSEDLADYFCQQYSNYPTFGAGTKLELKR

SEQ ID NO: 261 - DNA, 321 bp, Mus musculus
gacattgtgatgacccagtctcacaaattcatgtccacatcagtaggaggcagggtcagcatcacctgcaaggccagtcaggatgtggatacttctgtagcctggtatcaacagaaaccagggcaagctcctaaacttctggtttactgggcatccacccgccacactggagtccctgatcgcttcacaggcagtggatctgggacagatttcactctcaccattagcaatgtgcagtctgaagacttggcagattatttctgtcagcaatatagcaactatcccacgttcggtgctgggaccaagctggaactgaaacgt

SEQ ID NO: 262 - protein, 120 aa, Mus musculus
EVQLQQSGAELVRPGASVKLSCKASGFNIKDYYMHGVKHRPEQGLEWIGRIDPENGNTIYDPKFQGKATITADTSSNTAYLQLSSLTSEDTAVYYCARSPYDYHAWFAYWGQGTLVTVSS

SEQ ID NO: 263 - DNA, 360 bp, Mus musculus
gaggttcagctgcagcagtctggggctgagcttgtgaggccaggggcctcagtcaagttgtcctgcaaagcttctggcttcaacattaaagactactatatgcacggggtgaaacataggcctgaacagggcctggagtggattggaaggattgatcctgaaaatggtaatactatatatgacccgaagttccagggcaaggccactataacagcagacacatcctccaacacagcctacctgcagctcagcagcctgacatctgaggacactgccgtctattactgtgctagatccccttatgattaccacgcctggtttgcttactggggccaagggactctggtcaccgtctcgagc

SEQ ID NO: 264 - protein, 108 aa, Mus musculus
DIKMTQSPSSMYTSLGERVTITCKASQDISNYFTWFQQKPGKSPKTLIYRANRLVDGVPSRFSGSGSGQDFSLTISSLEYEDMGIYYCLQYDEFPYTFGGGTKLELKR

SEQ ID NO: 265 - DNA, 324 bp, Mus musculus
gacatcaagatgacccagtctccatcttccatgtatacatctctaggcgagagagtcactatcacttgcaaggcgagtcaggacattagtaactattttacctggttccagcagaaaccagggaaatctcctaagaccctgatctatcgtgcaaacagattggtagatggggtcccatcaaggttcagtggcagtggatctgggcaagatttttctctcaccatcagcagcctcgaatatgaagatatgggaatttattattgtctacagtatgatgagtttccgtacacgttcggaggggggaccaagttggagctcaaacgt

SEQ ID NO: 266 - protein, 117 aa, Mus musculus
EVKLVESGGGVMQPGGSLKLSCAASGFTFRTYTMSWVRQTPEKRLEWVAYISDGGGSSYFPDTVKGRFTVSRDNAKNTLYLQMSSLRSEDTAMYYCTRHSNWYFDVWGAGTSVTVSS

SEQ ID NO: 267 - DNA, 351 bp, Mus musculus
gaagtgaagttggtggagtctgggggaggtgtaatgcagcctggagggtccctgaaactctcctgtgcagcctctgggttcactttcagaacctataccatgtcttgggttcgccagactccagagaagaggctggagtgggtcgcatatattagtgatggtggtggtagttcctactttccagacactgtcaagggccgattcaccgtctccagggacaatgccaagaacaccctatacctgcaaatgagcagtctgaggtctgaggacacggccatgtattactgtacaagacattctaactggtacttcgatgtctggggcgcagggacctcagtcaccgtctcgagc

SEQ ID NO: 268 - protein, 108 aa, Mus musculus
DIQMTQTTSSLSASLGDRVTIRCRASQDISNYLNWYQQKPDGPVKLLIYYTSTLTSGVPSRFSGSGSGTDYSLTISNLDQEDIATYFCQQGKTFPFTFGSGTKLEIKR

SEQ ID NO: 269 - DNA, 324 bp, Mus musculus
gatatccagatgacacagactacatcctccctgtctgcctctctgggagacagagtcaccatccgttgcagggcaagtcaggacattagcaattatttaaactggtatcagcagaaaccagatggacctgttaaactcctgatctactacacatctacattgacctcaggagtcccatcaaggttcagtggcagtgggtctggaacagattattctctcaccattagcaacctggaccaagaagatattgccacttacttttgccaacagggtaagacgtttccattcacgttcggctcggggacaaagttggaaataaaacgt

SEQ ID NO: 270 - protein, 120 aa, Mus musculus
QVQLQQSGAELVRPGTSVKVSCKASGYAFTNYFIEWVKQRPGQGLEWIGAINPGSGGTNYNERFKGKATLSADKSSSTAYMQLSSLTSDDSAVYFCAREDYGDVYAMDYWGQGTSVTVSS

SEQ ID NO: 271 - DNA, 360 bp, Mus musculus
caggtccagctgcagcagtctggagctgagctggtcaggcctgggacttcagtgaaggtgtcctgcaaggcttctggatacgccttcactaattacttcatagagtgggtaaaacagaggcctggacagggccttgagtggattggagcgattaatcctggaagtggtggtactaactacaatgagaggttcaagggcaaggcaactctgtctgcagacaaatcctccagcactgcctacatgcagctcagcagcctgacatctgatgactctgcggtctatttctgtgcaagagaggactatggtgatgtctatgctatggactactggggtcaaggaacctcggtcacagtctcgagc

SEQ ID NO: 272 - protein, 107 aa, Mus musculus
DIVMTQSHKFMSTSVGDRVSITCKASQDVDTSVAWYQQKPGQAPKLLIYWASTRHTGVPDRFTGSGSGTDFTLTISNVQSEDLADYFCQQYSSYPTFGAGTKLELKR

SEQ ID NO: 273 - DNA, 321 bp, Mus musculus
gacattgtgatgacccagtctcacaaattcatgtccacatcagtaggagacagggtcagcatcacctgcaaggccagtcaggatgtggatacttctgtagcctggtatcaacagaaaccagggcaagctcctaaactactgatttactgggcatccacccggcacactggagtccctgatcgcttcacaggcagtggatctgggacagatttcactctcaccattagcaatgtgcagtctgaagacttggcagattatttctgtcagcaatatagcagctatcccacgttcggtgctgggaccaagctggaactgaaacgt

SEQ ID NO: 274 - protein, 120 aa, Mus musculus; identical to SEQ ID NO: 262

SEQ ID NO: 275 - DNA, 360 bp, Mus musculus
gaggttcagctgcagcagtctggggctgagcttgtgaggccaggggcctcagtcaagttgtcctgcaaagcttctggcttcaacattaaagactactatatgcacggggtgaaacataggcctgaacagggcctggagtggattggaaggattgatcctgagaatggtaatactatatatgacccgaagttccagggcaaggccactataacagcagacacatcctccaacacagcctacctgcagctcagcagcctgacatctgaggacactgccgtctattactgtgctagatccccttatgattaccacgcctggtttgcttactggggccaagggactctggtcactgtctcgagc

SEQ ID NO: 276 - protein, 107 aa, Mus musculus
DIQMTQSPSSLSASVGDRVTITCKASQDVDTSVAWYQQKPGKAPKLLIYWASTRHTGVPDRFSGSGSGTDFTLTISSLQPEDFATYYCQQYSSYPTFGQGTKLEIKR

SEQ ID NO: 277 - DNA, 321 bp, Mus musculus
gatatccagatgacccagtctccatcctccctgtctgcatctgtaggcgaccgtgtcaccatcacttgcaaggcaagtcaggacgttgatacctctgtagcttggtatcagcagaaaccagggaaagcccctaagctcctgatctattgggcatccactcgtcatactggggtcccagaccgcttcagtggcagtggttctgggacagatttcactctcaccatcagcagtctgcaacctgaagattttgcaacttattactgtcagcaatatagcagttaccctacgttcggtcaggggaccaagctggaaattaaacgt

SEQ ID NO: 278 - protein, 120 aa, Mus musculus
EVQLVQSGAEVKKPGATVKISCKASGFNIKDYYMHGVQHAPGKGLEWIGRIDPENGNTIYDPKFQGRVTITADTSTNTAYMELSSLRSEDTAVYYCARSPYDYHAWFAYWGQGTLVTVSS

SEQ ID NO: 279 - DNA, 360 bp, Mus musculus
gaggtccagctggtacagtctggggctgaggtgaagaagcctggggctacagtgaaaatctcctgcaaggcttctggattcaacatcaaagactactacatgcacggggtgcaacatgcccctggaaaagggcttgagtggattggacgtattgatcctgaaaatggtaacacaatctacgacccgaagttccagggccgcgtcaccattaccgcggacacgtctacaaacacagcctacatggagctgagcagcctgcgttctgaggacacggccgtgtattactgtgcacgttctccgtatgattatcacgcctggtttgcttactggggccaagggactctggtcaccgtctcgagc

SEQ ID NO: 280 - protein, 112 aa, Mus musculus
DIVLTQSPASLAVSLGQRATIYCKASQSVDYDGDSYMNWYQQKPGQPPKVLIFAASNLESGIPARFSGSRSGTDFTLNIHPVEEEDAATYYCQQTNEDPPTFGGGTKLELKR

SEQ ID NO: 281 - DNA, 336 bp, Mus musculus
gacattgtgctgacccaatctccagcttctttggctgtgtctctagggcagagggccaccatttactgcaaggccagccaaagtgttgattatgatggtgatagttatatgaactggtaccaacagaaaccaggacagccacccaaagtcctcatctttgctgcatccaatctagaatctgggatcccggccaggtttagtggcagtagatctgggacagacttcaccctcaacatccatcctgtggaggaggaggatgctgcaacctattactgtcaacaaactaatgaggatcctccgacgttcggtggcggcaccaagctggaactgaaacgt

SEQ ID NO: 282 - protein, 119 aa, Mus musculus
QVQLQQSGAELVKPGASVKLSCKASGYAFISFWIHWVKQRPGQGLEWIGEINPSNGRTDYNAKFKTKATLTVDRSSSTAYMQLSSLTSEDSADYYCARGGTGTWYFDVWGAGTTVTVSS

SEQ ID NO: 283 - DNA, 357 bp, Mus musculus
caggtccaactgcagcagtctggggctgaactggtgaagcctggggcttcagtgaagctgtcctgcaaggcttctggctacgcctttattagcttctggatacactgggtgaagcagaggcctggtcaaggccttgagtggattggagagattaatcctagcaacggtcgtactgactacaatgcgaagttcaagaccaaggccacactgactgttgacagatcctcctccacagcctacatgcaactcagcagcctgacatctgaggactctgcggactattactgtgcaagaggaggaactgggacctggtacttcgatgtctggggcgcagggaccacagtcaccgtctcgagc

SEQ ID NO: 284 - protein, 113 aa, Mus musculus
DVVMTQTPLTLSVTIGQPASISCKSSQSLLDSDGETYLNWLLQRPGQSPKRLIYLVSKLDSGVPDRFTGSGSGTDFTLKISRVETEDLGVYYCWQGTHFPYTFGGGTKLELKR

SEQ ID NO: 285 - DNA, 339 bp, Mus musculus
gatgttgtgatgacccagactccactcactttgtcggttaccattggacaaccagcctccatctcttgcaagtcaagtcagagcctcttagatagtgatggagagacatatttgaattggttgttacagaggccaggccagtctccaaagcgcctaatctatctggtgtctaaactggactctggagtccctgacaggttcactggcagtggatcagggacagatttcacactgaaaatcagcagagtggagactgaggatttgggagtttattattgctggcaaggtacacattttccgtacacgttcggaggggggaccaagctggaactgaaacgt

SEQ ID NO: 286 - protein, 116 aa, Mus musculus
EVKLEESGGGLVQPGGSMKLSCVASGFTFSNYWMNWVRQSPEKGLEWVAEIRLKSDNYATHFAESVKGRFTISRDDSKSSVYLQMNNLRAEDTGIYYCTGILFGYWGQGTLVTVSS

SEQ ID NO: 287 - DNA, 348 bp, Mus musculus
gaagtgaaacttgaggagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctgataattatgcaacacattttgcggagtctgtgaaagggaggttcaccatctcaagagatgattccaaaagtagtgtctacctgcaaatgaacaacttaagagctgaagacactggcatttattactgtacgggtatcctctttggttactggggccaagggactctggtcactgtctcgagc

SEQ ID NO: 288 - protein, 112 aa, Mus musculus
DIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPWTFGGGTKLELKR

SEQ ID NO: 289 - DNA, 336 bp, Mus musculus
gacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgctacctattactgtcagcaaagtaatgaggatccgtggacgttcggtggaggcaccaagctggagctgaaacgt

SEQ ID NO: 290 - protein, 117 aa, Mus musculus
QVQLLQPGAELVKPGASVKLSCKASGYTFTSYWMHWVKQRPGQGLEWIGEINPSNGRTDYNENFKSKATLTVDKSSSTAYMQLSSLTSEDSAVYYCARGGVYAMDYWGQGTSVTVSS

SEQ ID NO: 291 - DNA, 351 bp, Mus musculus
caggtccaactgctgcagcctggggctgaactggtgaagcctggggcttcagtgaagctgtcctgcaaggcttctggctacaccttcaccagctactggatgcactgggtgaagcagaggcctgggcaaggccttgagtggattggagagattaatcctagcaacggtcgtactgactacaatgagaacttcaagagcaaggccacactgactgtagacaaatcctccagcacagcctacatgcaactcagcagcctgacatctgaggactctgcggtctattactgtgcaagagggggggtctatgctatggactactggggtcaaggaacctcagtcaccgtctcgagc

SEQ ID NO: 292 - protein, 113 aa, Mus musculus; identical to SEQ ID NO: 244

SEQ ID NO: 293 - DNA, 339 bp, Mus musculus; identical to SEQ ID NO: 245

SEQ ID NO: 294 - protein, 116 aa, Mus musculus
EIQLQQSGTELVRSGASVKLSCTASGFNIKDYYIHWVRQRPDQGLEWIGWIDPENGDTEYAPKFQDRATLTADTSSNTAYLQLTLLTSEDTAVYYCNEGSGLIPYWGQGTPVTVSS

SEQ ID NO: 295 - DNA, 348 bp, Mus musculus
gagattcagctgcagcagtctgggacagagcttgtgaggtcaggggcctcagtcaaattgtcctgcacagcttctggcttcaacattaaagactactatattcactgggtgagacagaggcctgaccagggcctggagtggattggatggattgatcctgagaatggtgatactgaatatgccccgaagttccaggacagggccactttgactgcagacacatcctccaacacagcctacctgcagctcacactcctgacatctgaggacactgccgtctattactgtaatgagggctcgggcttgattccttactggggccaagggactccggtcaccgtctcgagc

SEQ ID NO: 296 - protein, 108 aa, Mus musculus; identical to SEQ ID NO: 268

SEQ ID NO: 297 - DNA, 324 bp, Mus musculus; identical to SEQ ID NO: 269

SEQ ID NO: 298 - protein, 120 aa, Mus musculus
QVQLQQSGAELVRPGTSVKVSCKASGYVFINYFIEWVKQRPGQGLEWIGVINPENGGTNYNERFKDKAALTADTSSSTAYMQLSSLTSDDSAVYFCAREDYGDVYAMDYWGQGTSVTVSS

SEQ ID NO: 299 - DNA, 360 bp, Mus musculus
caggtccagctgcagcagtctggagctgaactggtcaggcctgggacttcagtgaaggtgtcctgcaaggcttctggatacgtcttcattaattacttcatagagtgggttaaacagaggcctggacagggccttgagtggattggagtgattaatcctgaaaatggtggtactaactacaatgagagattcaaggacaaggcagcactgactgcagacacatcctccagcactgcctacatgcaactcagcagcctgacatctgatgactctgcggtctatttctgtgcaagagaggactatggtgatgtctatgctatggactactggggtcaaggaacctcagtcacagtctcgagc

SEQ ID NO: 300 - protein, 113 aa, Mus musculus; identical to SEQ ID NO: 244

SEQ ID NO: 301 - DNA, 339 bp, Mus musculus; identical to SEQ ID NO: 245

SEQ ID NO: 302 - protein, 116 aa, Mus musculus
EVQLQQSGTELVRSGASVRLSCTASGFNIKDYYIHWVKQRPEQGLEWIGWIDPENGDSEYAPKFQDKATMTADTSSNTAYLQLSSLTSEDTAVYYCNEGSGLIPYWGHGTLVTVSS

SEQ ID NO: 303 - DNA, 348 bp, Mus musculus
gaggttcagctgcagcagtctgggacagagcttgtgaggtcaggggcctcagtcaggttgtcctgcacagcttctggcttcaacattaaagactactatatacactgggtgaaacagaggcctgaacagggcctggagtggattggatggattgatcctgagaatggtgattctgaatatgccccgaagttccaggacaaggccaccatgactgcagacacatcctccaacacagcctacctgcagctcagcagcctgacatctgaggacactgccgtctattattgtaatgagggctcgggcttgattccttactggggccacgggactctggtcacagtctcgagc

SEQ ID NO: 304 - protein, 113 aa, Mus musculus
DIVLTQTPLSLPVSLGDHASISCRSSQSLVHSNGDVYFHWYLQKPGQSPKLLIYEVSNRFSGVPDRFSGSGSGTYFTLKISRVEAEDLGVYFCSQSTHVPYTFGGGTKLELKR

SEQ ID NO: 305 - DNA, 339 bp, Mus musculus
gatattgtgttgacccaaactcctctctctctgcctgtcagtcttggagatcatgcctccatctcttgcagatctagtcagagccttgtacacagtaatggcgacgtctatttccattggtacctgcagaagccaggccagtctccaaagctcctgatctacgaagtttccaaccgattttctggggtcccagacaggttcagtggcagtggatcagggacatatttcacactcaagatcagcagagtggaggctgaggatctgggagtttatttctgctctcaaagtacacatgttccgtacactttcggaggggggaccaagttggagctcaaacgt

SEQ ID NO: 306 - protein, 116 aa, Mus musculus; identical to SEQ ID NO: 302

SEQ ID NO: 307 - DNA, 348 bp, Mus musculus; identical to SEQ ID NO: 303

SEQ ID NO: 308 - protein, 112 aa, Mus musculus
DVVLTQTPLSLPVSLGDQASISCRSSQSLVHSNGDVYLHWYLQKPGQSPKLLIYEVSNRFSGVPDRFSGSGSGTDFTLKINRVEAEDLGVYFCSQSTHVPYTFGGGTKLELK

SEQ ID NO: 309 - DNA, 339 bp, Mus musculus
gatgttgtgttgacccaaactccactctccctgcctgtcagtcttggagatcaagcctccatctcttgcagatctagtcagagccttgtacacagtaatggagacgtctatttacattggtacctgcagaagccaggccagtctccaaagctcctgatctacgaagtttccaaccgattttctggggtcccagacaggttcagtggcagtggatcagggacagatttcacactcaagatcaacagagtggaggctgaggatctgggagtttatttctgctctcaaagtacacatgttccgtacacgttcggaggggggaccaagctggaactgaaacgt

SEQ ID NO: 310 - protein, 116 aa, Mus musculus
EVQLQQSGTELVRSGASVKLSCTASGFNIKDYYVHWVKQRPEQGLEWIGWIDPDNGDSEYAPKFQDKATMTADTSSNTAYLQLNSLTSEDTAVYYCNEGSGLIPYWGPGTLVTVSS

SEQ ID NO: 311 - DNA, 348 bp, Mus musculus
gaggttcagctgcagcagtctgggacagagcttgtgaggtcaggggcctcagtcaagttgtcctgcacagcttctggcttcaacattaaagactactatgtacactgggtgaagcagaggcctgaacagggcctggaatggattggatggattgatcctgacaatggtgatagtgaatatgccccgaagttccaggacaaggccactatgactgcagacacatcctccaacacagcctaccttcaactcaacagcctgacttctgaggacactgccgtctattattgtaatgagggctcgggcttgattccttactggggcccagggactctggtcacagtctcgagc

SEQ ID NO: 312 - protein, 113 aa, Mus musculus
DVVMTQTPLSLPVSLGDQASISCRSSQSLVHSNGDVYLHWYLQKPGQSPKLLIYEVSNRFSGVPDRFSGSGSGTDFTLKISRVEAEDLGVYFCSQTTHVPYTFGGGTKLELKR

SEQ ID NO: 313 - DNA, 339 bp, Mus musculus
gatgttgtgatgacccaaactccactctccctgcctgtcagtcttggagatcaagcctccatctcctgcagatctagtcagagccttgtacacagtaatggagacgtctatttacattggtacctgcagaagccaggccagtctccaaaactcctgatctacgaagtttccaaccgattttctggggtcccagacaggttcagtggcagtggttcagggacagatttcacactcaagatcagcagagtggaggctgaggatctgggagtttatttctgttctcaaactacacatgttccgtacacgttcggaggggggaccaagctggagctgaaacgt

SEQ ID NO: 314 - protein, 116 aa, Mus musculus; identical to SEQ ID NO: 294

SEQ ID NO: 315 - DNA, 348 bp, Mus musculus; identical to SEQ ID NO: 295

SEQ ID NO: 316 - protein, 113 aa, Mus musculus
DVVMTQTPLSLPVSLGDQASISCRSSQSLVHSNGDVYLHWYLQKPGQSPKLLIYEVSNRFSGVPDRFTGSGSGTDFTLKISRVEAEDLGVFFCSQSTHVPYTFGGGTKLELKR

SEQ ID NO: 317 - DNA, 339 bp, Mus musculus
gatgttgtgatgacccaaactccactctccctgcctgtcagtcttggagatcaagcctccatctcctgcagatctagtcagagccttgtacacagtaatggagacgtctatttacattggtacctgcagaagccaggccagtctccaaaactcctgatctacgaagtttccaaccgattttctggggtcccagacaggttcactggcagtggttcagggacagatttcacactcaagatcagcagagtggaggctgaggatctgggagtttttttctgctctcaaagtacacatgttccgtacacgttcggaggggggaccaagctggaactgaaacgt

SEQ ID NO: 318 - protein, 116 aa, Mus musculus
EIQLQQSGTELVRSGASVKLSCTASGFNIKDYYIHWVKQRPDQGLEWIGWIDPENGDTEYAPKFQDRATMTADTSSNTAYLQLTILTSEDTAVYYCNEGSGLIPYWGQGTLVTVSS

SEQ ID NO: 319 - DNA, 348 bp, Mus musculus
gagattcagctgcagcagtctgggacagagcttgtgaggtcaggggcctcagtcaaattgtcctgcacagcttctggcttcaacattaaagactactatattcactgggtgaaacagaggcctgaccagggcctggagtggattggatggattgatcctgagaatggtgatactgaatatgccccgaagttccaggacagggccactatgactgcagacacatcctccaacacagcctacctgcagctcaccatcctgacatctgaggacactgccgtctattactgtaatgagggctcgggcttgattccttactggggccaagggactctggtcaccgtctcgagc

SEQ ID NO: 320 - protein, 108 aa, Mus musculus
DIQMNQSPSSLSASLGDTITITCHASQNINVWLSWYQQKPGNIPKLLIYKVSNLHTGVPSRFSGSGSGTGFTLTISSLQPEDIATYYCQQGQSYPLTFGGGTKLELKR

SEQ ID NO: 321 - DNA, 324 bp, Mus musculus
gacatccagatgaaccagtctccatccagtctgtctgcatcccttggagacacaattaccatcacttgccatgccagtcagaacattaatgtttggttaagctggtaccagcagaaaccaggaaatattcctaaactattgatctataaggtttccaacttgcacacaggcgtcccatcaaggtttagtggcagtggatctggaacaggtttcacattaaccatcagcagcctgcagcctgaagacattgccacttactactgtcaacagggtcaaagttatcctctgacgttcggtggaggcaccaagttggagctcaaacgt

SEQ ID NO: 322 - protein, 120 aa, Mus musculus
EVQLQQSGAELVRPGALVKLSCKASGFNIKDYYIHWVKQRPEQGLEWIGRIDPENGNTIYDPKFQGKAIITADTSSNTAYLQLSSLTSEDTAVYYCARCDNDPGSEMDYWGQGTTVTVSS

SEQ ID NO: 323 - DNA, 360 bp, Mus musculus
gaggttcagctgcagcagtctggggctgagcttgtgaggccaggggccttagtcaaattgtcctgcaaagcttctggattcaacattaaagactactatatacactgggtgaagcagaggcctgaacagggcctggagtggattggaaggattgatcctgaaaatggtaatactatatatgacccgaagttccagggcaaggccattataacagcagacacatcctccaacacagcctacctgcagctcagcagcctgacatctgaggacactgccgtctattactgtgctagatgtgataacgaccccggctctgaaatggactactggggtcaaggaaccacggtcaccgtctcgagc

SEQ ID NO: 324 - protein, 113 aa, Mus musculus; identical to SEQ ID NO: 304

SEQ ID NO: 325 - DNA, 339 bp, Mus musculus; identical to SEQ ID NO: 305

SEQ ID NO: 326 - protein, 116 aa, Mus musculus; identical to SEQ ID NO: 310

SEQ ID NO: 327 - DNA, 348 bp, Mus musculus; identical to SEQ ID NO: 311

(19) United States
(12) Patent Application Publication
(10) Pub. No.: US 2010/0036091 A1
(43) Pub. Date: Feb. 11, 2010

(54) ANTIBODY-BASED DIAGNOSTICS AND THERAPEUTICS

(75) Inventors: Martyn Kim Robinson, Buckinghamshire (GB); Christopher J. Paszty, Ventura, CA (US); Alastair James Henry, Middlesex (GB); Alistair Lawson, Alresford (GB); Andy Popplewell, Berkshire (GB)

Correspondence Address: MARSHALL, GERSTEIN & BORUN LLP, 233 SOUTH WACKER DRIVE, 6300 SEARS TOWER, CHICAGO, IL, 60606-6357, US

(73) Assignees: AMGEN INC., Thousand Oaks, CA (US); UCB PHARMA S.A., Slough, Berkshire (GB)

(21) Appl. No.: 12/513,809

(22) PCT Filed: Nov. 9, 2007
(86) PCT No.: PCT/US2007/084276

§ 371 Date: May 6, 2009

Related U.S. Application Data
(60) Provisional application No. 60/857,882, filed on Nov. 10, 2006.

(51) Int. Cl. C07K 7/08; C12P 21/06; C12N 5/00; C12N 15/63; C07K 7/06; C07K 16/00; C07H 21/04

(52) U.S. Cl. 530/326; 435/69.1; 435/325; 435/320.1; 530/327; 530/328; 530/329; 530/330; 530/387.9; 536/23.1

(57) ABSTRACT

Compositions and methods relating to sclerostin binding agents, such as antibodies and polypeptides capable of binding to sclerostin, are provided.

### TECHNICAL FIELD

The present invention relates generally to epitopes of sclerostin protein, including human sclerostin protein, and binding agents (such as antibodies) capable of binding to sclerostin or fragments thereof.

### BACKGROUND OF THE INVENTION

Two or three distinct phases of changes to bone mass occur over the life of an individual (see Riggs, West J. Med., 154:63-77 (1991)). The first phase occurs in both men and women and proceeds to attainment of a peak bone mass. This first phase is achieved through linear growth of the endochondral growth plates and radial growth due to a rate of periosteal apposition. The second phase begins around age 30 for trabecular bone (flat bones such as the vertebrae and pelvis) and about age 40 for cortical bone (e.g., long bones found in the limbs) and continues to old age. This phase is characterized by slow bone loss and occurs in both men and women. In women, a third phase of bone loss also occurs, most likely due to postmenopausal estrogen deficiencies. During this phase alone, women may lose an additional bone mass from the cortical bone and from the trabecular compartment (see Riggs, supra).

Loss of bone mineral content can be caused by a wide variety of conditions and may result in significant medical problems. For example, osteoporosis is a debilitating disease in humans and is characterized by marked decreases in skeletal bone mass and mineral density, structural deterioration of bone, including degradation of bone microarchitecture and corresponding increases in bone fragility (i.e., decreases in bone strength), and susceptibility to fracture in afflicted individuals. Osteoporosis in humans is generally preceded by clinical osteopenia (bone mineral density that is greater than one standard deviation but less than 2.5 standard deviations below the mean value for young adult bone), a condition found in approximately 25 million people in the United States. Another 7-8 million patients in the United States have been diagnosed with clinical osteoporosis (defined as bone mineral content greater than 2.5 standard deviations below that of mature young adult bone). The frequency of osteoporosis in the human population increases with age. Among Caucasians, osteoporosis is predominant in women who, in the United States, comprise 80% of the osteoporosis patient pool. The increased fragility and susceptibility to fracture of skeletal bone in the aged is aggravated by the greater risk of accidental falls in this population. Fractured hips, wrists, and vertebrae are among the most common injuries associated with osteoporosis. Hip fractures in particular are extremely uncomfortable and expensive for the patient, and for women, correlate with high rates of mortality and morbidity.

Although osteoporosis has been regarded as an increase in the risk of fracture due to decreased bone mass, few of the presently available treatments for skeletal disorders can increase the bone density of adults, and most of the presently available treatments work primarily by inhibiting further bone resorption rather than stimulating new bone formation. Estrogen is now being prescribed to retard bone loss. However, some controversy exists over whether patients gain any long-term benefit and whether estrogen has any effect on patients over 75 years old. Moreover, use of estrogen is believed to increase the risk of breast and endometrial cancer. Calcitonin, osteocalcin with vitamin K, or high doses of dietary calcium, with or without vitamin D, have also been suggested for postmenopausal women. High doses of calcium, however, often have undesired gastrointestinal side effects, and serum and urinary calcium levels must be continuously monitored (e.g., Khosla and Riggs, Mayo Clin. Proc., 70:978982 (1995)).

Other current therapeutic approaches to osteoporosis include bisphosphonates (e.g., Fosamax™, Actonel™, Bonviva™, Zometa™, olpadronate, neridronate, skelid, bonefos), parathyroid hormone, calcilytics, calcimimetics (e.g., cinacalcet), statins, anabolic steroids, lanthanum and strontium salts, and sodium fluoride. Such therapeutics, however, are often associated with undesirable side effects (see Khosla and Riggs, supra).

Sclerostin, the product of the SOST gene, is absent in sclerosteosis, a skeletal disease characterized by bone overgrowth and strong dense bones (Brunkow et al., Am. J. Hum. Genet., 68:577-589 (2001); Balemans et al., Hum. Mol. Genet., 10:537-543 (2001)). The amino acid sequence of human sclerostin is reported by Brunkow et al. ibid and is disclosed herein as SEQ ID NO:1.

### BRIEF SUMMARY OF THE INVENTION

The invention relates to an isolated antibody selected from the group consisting of antibodies AA, BB, CC, DD, EE, FF, GG, HH, II, JJ, KK, LL, MM, NN, OO, PP, QQ, RR, SS, TT, UU, VV, and WW; the isolated antibody, or an antigen-binding fragment thereof, may be a polyclonal antibody, a monoclonal antibody, a humanized antibody, a human antibody, or a human/non-human chimeric antibody, such as a mouse/human or rabbit/human chimeric antibody.

The invention further relates to a methods for detecting, diagnosing, and determining the progression or regression of a bone disorder associated with at least one of low bone mass, low bone mineral density, and poor bone quality in a mammalian subject which comprises obtaining a biological sample from a subject suspected of suffering from the disorder, contacting the biological sample with an agent capable of detecting sclerostin, and identifying or quantitating a binding complex between the agent and sclerostin, wherein the agent comprises an anti-sclerostin antibody, or sclerostin-binding fragment thereof.

Provided herein are antibodies that specifically bind to human sclerostin. The antibodies can be characterized by their ability to bind to human sclerostin or a fragment thereof.

Also provided is an isolated antibody, or an antigen-binding fragment thereof, that specifically binds to human sclerostin and has at least one CDR sequence selected from SEQ ID NO:101, 104, 110, 116, 122, 128, 134, 140, 146, 152, 158, 164, 170, 176, 182, 188, 194, 200, 206, 212, 218, 224, and 230 for CDR-L1; SEQ ID NO:102, 105, 111, 117, 123, 129, 135, 141, 147, 153, 159, 165, 171, 177, 183, 189, 195, 201, 207, 213, 219, 225, 231 for CDR-L2; SEQ ID NO:103, 106, 112, 118, 124, 130, 136, 142, 148, 154, 160, 166, 172, 178, 184, 190, 196, 202, 208, 214, 220, 226, and 232 for CDR-L3; SEQ ID NO: 98, 107, 113, 119, 125, 131, 137, 143, 149, 155, 161, 167, 173, 179, 185, 191, 197, 203, 209, 215, 221, 227, and 233 for CDR-H1; SEQ ID NO:99, 108, 114, 120, 132, 144, 150, 156, 162, 168, 174, 180, 186, 192, 198, 204, 210, 216, 222, 228, and 234 for CDR-H2; SEQ ID NO:100, 109, 115, 121, 127, 133, 139, 145, 151, 157, 163, 169, 175, 181, 187, 193, 199, 205, 211, 217, 223, 229, and 235 for CDR-H3; and variants thereof.

Further provided is a method for treating a bone disorder associated with at least one of low bone mass, low bone mineral density, and poor bone quality in a mammalian subject which comprises providing to a subject in need of such treatment an amount of an anti-sclerostin agent sufficient to modulate at least one of low bone mass, low bone mineral density, and poor bone quality wherein the anti-sclerostin agent comprises an antibody, or sclerostin-binding fragment thereof.

These and other aspects of the present invention will become apparent upon reference to the following detailed description and attached drawings. All references disclosed herein are hereby incorporated by reference in their entireties as if each was incorporated individually.

### BRIEF DESCRIPTION OF THE DRAWINGS

FIG. 1 depicts the amino acid sequence of the mature form (signal peptide cleaved off) of human sclerostin (SEQ ID NO:1). Also depicted is the nucleotide sequence of the human sclerostin coding region that encodes the mature form of human sclerostin. The eight cysteines are numbered C1 through C8. The cystine-knot is formed by three disulfide bonds (C1-C5; C3-C7; C4-C8). C2 and C6 also form a disulfide bond, however this disulfide is not part of the cystine-knot.

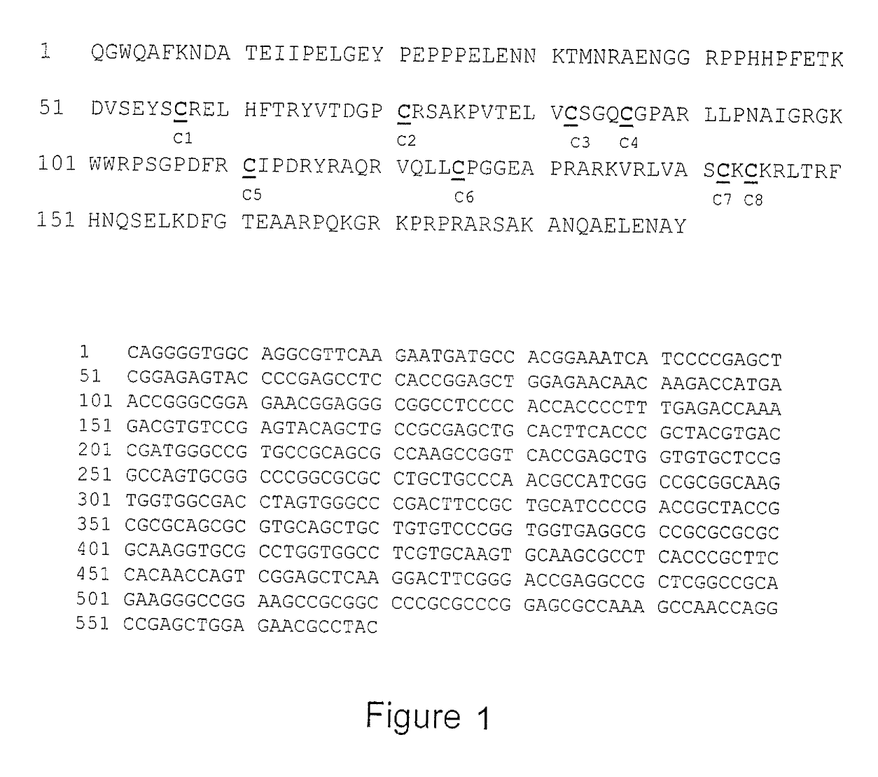

FIG. 2 depicts a schematic of the basic structure of human sclerostin. There is an N-terminal arm (from the first Q to C1) and a C-terminal arm (from C8 to the terminal Y). In between these arms there is the cystine-knot structure (formed by three disulfides: C1-C5; C3-C7; C4-C8) and three loops which are designated Loop 1, Loop 2 and Loop 3. The distal regions of Loop 1 and Loop 3 are linked by the C2-C6 disulfide. Potential trypsin cleavage sites are indicated (arginine=R and lysine=K). Some of the potential AspN cleavage sites are indicated [only aspartic acid (D) residues are shown].

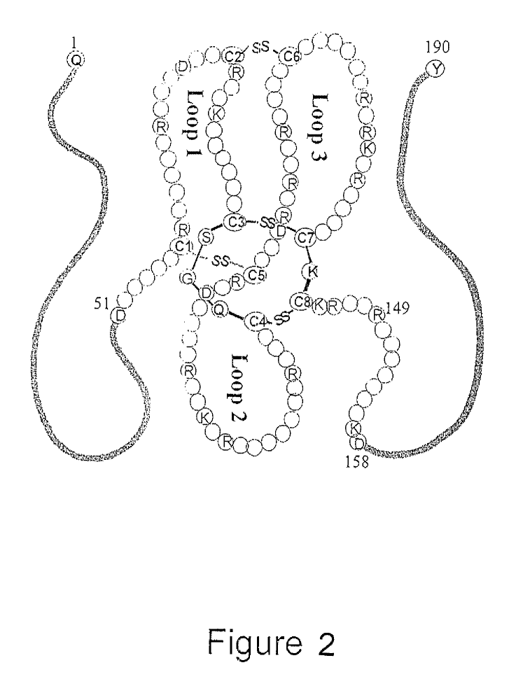

### DETAILED DESCRIPTION

The present invention relates to regions of the human sclerostin protein that contain epitopes recognized by antibodies that also bind to full-length sclerostin, and methods of making and using these epitopes. The invention also provides binding agents (such as antibodies) that specifically bind to sclerostin or portions of sclerostin, and methods for using such binding agents. The binding agents are useful to block or impair binding of human sclerostin to one or more ligand.

Recombinant human sclerostin/SOST is commercially available from R&D Systems (Minneapolis, Minn., USA; 2006 cat #1406-ST-025). Additionally, recombinant mouse sclerostin/SOST is commercially available from R&D Systems (Minneapolis, Minn., USA; 2006 cat#1589-ST-025). Research grade sclerostin binding monoclonal antibodies are commercially available from R&D Systems (Minneapolis, Minn., USA; mouse monoclonal: 2006 cat#MAB1406; rat monoclonal: 2006 cat#MAB1589). U.S. Pat. Nos. 6,395,511 and 6,803,453, and U.S. Patent Publications 2004/0009535 and 2005/0106683 refer to anti-sclerostin antibodies generally.

As used herein, the term human sclerostin is intended to include the protein of SEQ ID NO:1 and allelic variants thereof. Sclerostin can be purified from 293T host cells that have been transfected by a gene encoding sclerostin by elution of filtered supernatant of host cell culture fluid using a Heparin HP column, using a salt gradient. The preparation and further purification using cation exchange chromatography are described in Examples 1 and 2.

Binding agents of the invention are preferably antibodies, as defined herein. The term “antibody” refers to an intact antibody, or a binding fragment thereof. An antibody may comprise a complete antibody molecule (including polyclonal, monoclonal, chimeric, humanized, or human versions having full length heavy and/or light chains), or comprise an antigen binding fragment thereof. Antibody fragments include F(ab′)₂, Fab, Fab′, Fv, Fc, and Fd fragments, and can be incorporated into single domain antibodies, single-chain antibodies, maxibodies, minibodies, intrabodies, diabodies, triabodies, tetrabodies, v-NAR and bis-scFv (see e.g., Hollinger and Hudson, Nature Biotechnology, 23(9):1126-1136 (2005)). Antibody polypeptides are also disclosed in U.S. Pat. No. 6,703,199, including fibronectin polypeptide monobodies. Other antibody polypeptides are disclosed in U.S. Patent Publication 2005/0238646, which are single-chain polypeptides.

Antigen binding fragments derived from an antibody can be obtained, for example, by proteolytic hydrolysis of the antibody, for example, pepsin or papain digestion of whole antibodies according to conventional methods. By way of example, antibody fragments can be produced by enzymatic cleavage of antibodies with pepsin to provide a 5S fragment termed F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using papain produces two monovalent Fab fragments and an Fc fragment directly. These methods are described, for example, by Goldenberg, U.S. Pat. No. 4,331,647, Nisonoff et al., Arch. Biochem. Biophys., 89:230 (1960); Porter, Biochem. J., 73:119 (1959); Edelman et al., in Methods in Enzymology, 1:422 (Academic Press 1967); and by Andrews, S. M. and Titus, J. A. in Current Protocols in Immunology (Coligan J. E., et al., eds.), John Wiley & Sons, New York (2003), pages 2.8.1-2.8.10 and 2.10A.1-2.10A.5. Other methods for cleaving antibodies, such as separating heavy chains to form monovalent light-heavy chain fragments (Fd), further cleaving of fragments, or other enzymatic, chemical, or genetic techniques may also be used, so long as the fragments bind to the antigen that is recognized by the intact antibody.

An antibody fragment may also be any synthetic or genetically engineered protein. For example, antibody fragments include isolated fragments consisting of the light chain variable region, “Fv” fragments consisting of the variable regions of the heavy and light chains, and recombinant single chain polypeptide molecules in which light and heavy variable regions are connected by a peptide linker (scFv proteins).

Another form of an antibody fragment is a peptide comprising one or more complementarity determining regions (CDRs) of an antibody. CDRs (also termed “minimal recognition units,” or “hypervariable region”) can be obtained by constructing polynucleotides that encode the CDR of interest. Such polynucleotides are prepared, for example, by using the polymerase chain reaction to synthesize the variable region using mRNA of antibody-producing cells as a template (see, for example, Larrick et al., Methods: A Companion to Methods in Enzymology 2:106, 1991; Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995); and Ward et al., “Genetic Manipulation and Expression of Antibodies,” in Monoclonal Antibodies: Principles and Applications, Birch et al., (eds.), page 137 (Wiley-Liss, Inc. 1995)).

Thus, in one embodiment, the binding agent comprises at least one CDR as described herein. The binding agent may comprise at least two, three, four, five or six CDR's as described herein. The binding agent further may comprise at least one variable region domain of an antibody described herein. The variable region domain may be of any size or amino acid composition and will generally comprise at least one CDR sequence responsible for binding to human sclerostin, for example CDR-H1, CDR-H2, CDR-H3 and/or the light chain CDRs specifically described herein and which is adjacent to or in frame with one or more framework sequences. In general terms, the variable (V) region domain may be any suitable arrangement of immunoglobulin heavy (V_{H}) and/or light (V_{L}) chain variable domains. Thus, for example, the V region domain may be monomeric and be a V_{H} or V_{L} domain, which is capable of independently binding human sclerostin with an affinity at least equal to 1×10⁻⁷M or less as described below. Alternatively, the V region domain may be dimeric and contain V_{H}-V_{H}, V_{H}-V_{L}, or V_{L}-V_{L}, dimers. The V region dimer comprises at least one V_{H} and at least one V_{L} chain that may be non-covalently associated (hereinafter referred to as F_{V}). If desired, the chains may be covalently coupled either directly, for example, via a disulfide bond between the two variable domains, or through a linker, for example, a peptide linker, to form a single chain Fv (scF_{V}).

The variable region domain may be any naturally occurring variable domain or an engineered version thereof. By engineered version is meant a variable region domain that has been created using recombinant DNA engineering techniques. Such engineered versions include those created, for example, from a specific antibody variable region by insertions, deletions, or changes in or to the amino acid sequences of the specific antibody. Particular examples include engineered variable region domains containing at least one CDR and optionally one or more framework amino acids from a first antibody and the remainder of the variable region domain from a second antibody.

The variable region domain may be covalently attached at a C-terminal amino acid to at least one other antibody domain or a fragment thereof. Thus, for example, a V_{H} domain that is present in the variable region domain may be linked to an immunoglobulin CH1 domain, or a fragment thereof. Similarly a V_{L} domain may be linked to a C_{K} domain or a fragment thereof. In this way, for example, the antibody may be an Fab fragment wherein the antigen binding domain contains associated V_{H} and V_{L} domains covalently linked at their C-termini to a CH1 and C_{K} domain, respectively. The CH1 domain may be extended with further amino acids, for example, to provide a hinge region or a portion of a hinge region domain as found in a Fab′ fragment, or to provide further domains, such as antibody CH2 and CH3 domains.

As described herein, binding agents comprise at least one of these CDRs. For example, one or more CDR may be incorporated into known antibody framework regions (IgG1, IgG2, etc.), or conjugated to a suitable vehicle to enhance the half-life thereof. Suitable vehicles include, but are not limited to Fc, polyethylene glycol (PEG), albumin, transferrin, and the like. These and other suitable vehicles are known in the art. Such conjugated CDR peptides may be in monomeric, dimeric, tetrameric, or other form. In one embodiment, one or more water-soluble polymer is bonded at one or more specific position, for example at the amino terminus, of a binding agent.

In certain preferred embodiments, a binding agent comprises one or more water soluble polymer attachments, including, but not limited to, polyethylene glycol, polyoxyethylene glycol, or polypropylene glycol. See, e.g., U.S. Pat. Nos. 4,640,835, 4,496,689, 4,301,144, 4,670,417, 4,791,192 and 4,179,337. In certain embodiments, a derivative binding agent comprises one or more of monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers. In certain embodiments, one or more water-soluble polymers are randomly attached to one or more side chains. In certain embodiments, PEG can act to improve the therapeutic capacity for a binding agent, such as an antibody. Certain such methods are discussed, for example, in U.S. Pat. No. 6,133,426, which is hereby incorporated by reference for any purpose.

It will be appreciated that a binding agent of the present invention may have at least one amino acid substitution, providing that the binding agent retains binding specificity. Therefore, modifications to the binding agent structures are encompassed within the scope of the invention. These may include amino acid substitutions, which may be conservative or non-conservative, that do not destroy the sclerostin binding capability of a binding agent. Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties. A conservative amino acid substitution may also involve a substitution of a native amino acid residue with a normative residue such that there is little or no effect on the polarity or charge of the amino acid residue at that position.

Non-conservative substitutions may involve the exchange of a member of one class of amino acids or amino acid mimetics for a member from another class with different physical properties (e.g., size, polarity, hydrophobicity, and/or charge). Such substituted residues may be introduced into regions of the human antibody that are homologous with non-human antibodies, or into the non-homologous regions of the molecule.

Moreover, one skilled in the art may generate test variants containing a single amino acid substitution at each desired amino acid residue. The variants can then be screened using activity assays known to those skilled in the art. Such variants could be used to gather information about other suitable variants. For example, if one discovered that a change to a particular amino acid residue resulted in destroyed, undesirably reduced, or unsuitable activity, variants with such a change may be avoided. In other words, based on information gathered from such routine experiments, one skilled in the art can readily determine the amino acids where further substitutions should be avoided either alone or in combination with other mutations.

A skilled artisan will be able to determine suitable variants of the polypeptide as set forth herein using well-known techniques. In certain embodiments, one skilled in the art may identify suitable areas of the molecule that may be changed without destroying activity by targeting regions not believed to be important for activity. In certain embodiments, one can identify residues and portions of the molecules that are conserved among similar polypeptides. In certain embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a protein that correspond to amino acid residues which are important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar polypeptides. In view of such information, one skilled in the art may predict the alignment of amino acid residues of an antibody with respect to its three dimensional structure. In certain embodiments, one skilled in the art may choose not to make radical changes to amino acid residues predicted to be on the surface of the protein, since such residues may be involved in important interactions with other molecules.

A number of scientific publications have been devoted to the prediction of secondary structure. See Moult J., Curr. Op. in Biotech., 7(4):422-427 (1996), Chou et al., Biochemistry, 13(2):222-245 (1974); Chou et al., Biochemistry, 113(2):211-222 (1974); Chou et al., Adv. Enzymol. Relat. Areas Mol. Biol., 47:45-148 (1978); Chou et al., Ann. Rev. Biochem., 47:251-276 and Chou et al., Biophys. J., 26:367-384 (1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The recent growth of the protein structural database (PDB) has provided enhanced predictability of secondary structure, including the potential number of folds within a polypeptide's or protein's structure. See Holm et al., Nucl. Acid. Res., 27(1):244-247 (1999). It has been suggested (Brenner et al., Curr. Op. Struct. Biol., 7(3):369-376 (1997)) that there are a limited number of folds in a given polypeptide or protein and that once a critical number of structures have been resolved, structural prediction will become dramatically more accurate.

Additional methods of predicting secondary structure include “threading” (Jones, D., Curr. Opin. Struct. Biol., 7(3):377-87 (1997); Sippl et al., Structure, 4(1):15-19 (1996)), “profile analysis” (Bowie et al., Science, 253:164-170 (1991); Gribskov et al., Meth. Enzym., 183:146-159 (1990); Gribskov et al., Proc. Nat. Acad. Sci., 84(13):4355-4358 (1987)), and “evolutionary linkage” (see Holm, supra (1999), and Brenner, supra (1997)).

In certain embodiments, variants of binding agents include glycosylation variants wherein the number and/or type of glycosylation site has been altered compared to the amino acid sequences of a parent polypeptide. In certain embodiments, variants comprise a greater or a lesser number of N-linked glycosylation sites than the native protein. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional preferred antibody variants include cysteine variants wherein one or more cysteine residues are deleted from or substituted for another amino acid (e.g., serine) as compared to the parent amino acid sequence. Cysteine variants may be useful when antibodies must be refolded into a biologically active conformation such as after the isolation of insoluble inclusion bodies. Cysteine variants generally have fewer cysteine residues than the native protein, and typically have an even number to minimize interactions resulting from unpaired cysteines.

Desired amino acid substitutions (whether conservative or non-conservative) can be determined by those skilled in the art at the time such substitutions are desired. In certain embodiments, amino acid substitutions can be used to identify important residues of antibodies to sclerostin, or to increase or decrease the affinity of the antibodies to sclerostin described herein.

According to certain embodiments, preferred amino acid substitutions are those which: (1) reduce susceptibility to proteolysis, (2) reduce susceptibility to oxidation, (3) alter binding affinity for forming protein complexes, (4) alter binding affinities, and/or (5) confer or modify other physiochemical or functional properties on such polypeptides. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al., Nature, 354:105 (1991), which are each incorporated herein by reference.

In certain embodiments, binding agents of the invention may be chemically bonded with polymers, lipids, or other moieties.

The binding agents may comprise at least one of the CDRs described herein incorporated into a biocompatible framework structure. In one example, the biocompatible framework structure comprises a polypeptide or portion thereof that is sufficient to form a conformationally stable structural support, or framework, or scaffold, which is able to display one or more sequences of amino acids that bind to an antigen (e.g., CDRs, a variable region, etc.) in a localized surface region. Such structures can be a naturally occurring polypeptide or polypeptide “fold” (a structural motif), or can have one or more modifications, such as additions, deletions or substitutions of amino acids, relative to a naturally occurring polypeptide or fold. These scaffolds can be derived from a polypeptide of any species (or of more than one species), such as a human, other mammal, other vertebrate, invertebrate, plant, bacteria, or virus.

Typically the biocompatible framework structures are based on protein scaffolds or skeletons other than immunoglobulin domains. For example, those based on fibronectin, ankyrin, lipocalin, neocarzinostain, cytochrome b, CP1 zinc finger, PST1, coiled coil, LACI-D1, Z domain and tendramisat domains may be used (see, e.g., Nygren and Uhlen, Current Opinion in Structural Biology, 7:463-469 (1997)).

In preferred embodiments, it will be appreciated that the binding agents of the invention include the humanized antibodies described herein. Humanized antibodies such as those described herein can be produced using techniques known to those skilled in the art (Zhang, W., et al., Molecular Immunology, 42(12):1445-1451 (2005); Hwang W. et al., Methods, 36(1):35-42 (2005); Dall'Acqua W F, et al., Methods, 36(1):43-60 (2005); and Clark, M., Immunology Today, 21(8):397-402 (2000)).

Additionally, one skilled in the art will recognize that suitable binding agents include portions of these antibodies, such as one or more of CDR-H1, CDR-H2, CDR-H3, CDR-L1, CDR-L2 and CDR-L3 as specifically disclosed herein. At least one of the regions of CDR-H1, CDR-H2, CDR-H3, CDR-L1, CDR-L2 and CDR-L3 may have at least one amino acid substitution, provided that the binding agent retains the binding specificity of the non-substituted CDR. The non-CDR portion of the binding agent may be a non-protein molecule, wherein the binding agent cross-blocks the binding of an antibody disclosed herein to sclerostin and/or neutralizes sclerostin. The non-CDR portion of the binding agent may be a non-protein molecule in which the binding agent exhibits a similar binding pattern to human sclerostin peptides in a “human sclerostin peptide epitope competition binding assay” as that exhibited by at least one of antibodies AA-WW, and/or neutralizes sclerostin. The non-CDR portion of the binding agent may be composed of amino acids, wherein the binding agent is a recombinant binding protein or a synthetic peptide, and the recombinant binding protein cross-blocks the binding of an antibody disclosed herein to sclerostin and/or neutralizes sclerostin. The non-CDR portion of the binding agent may be composed of amino acids, wherein the binding agent is a recombinant binding protein, and the recombinant binding protein exhibits a similar binding pattern to human sclerostin peptides in the human sclerostin peptide epitope competition binding assay (described hereinbelow) as that exhibited by at least one of the antibodies AA-WW, and/or neutralizes sclerostin.

Where an antibody comprises one or more of CDR-H1, CDR-H2, CDR-H3, CDR-L1, CDR-L2 and CDR-L3 as described above, it may be obtained by expression from a host cell containing DNA coding for these sequences. A DNA coding for each CDR sequence may be determined on the basis of the amino acid sequence of the CDR and synthesized together with any desired antibody variable region framework and constant region DNA sequences using oligonucleotide synthesis techniques, site-directed mutagenesis and polymerase chain reaction (PCR) techniques as appropriate. DNA coding for variable region frameworks and constant regions is widely available to those skilled in the art from genetic sequences databases such as GenBank®. Each of the above-mentioned CDRs will be typically located in a variable region framework at positions 31-35 (CDR-H1), 50-65 (CDR-H2) and 95-102 (CDR-H3) of the heavy chain and positions 24-34 (CDR-L1), 50-56 (CDR-L2) and 89-97 (CDR-L3) of the light chain according to the Kabat numbering system (Kabat et al., 1987 in Sequences of Proteins of Immunological Interest, U.S. Department of Health and Human Services, NIH, USA).

The present invention therefore relates to an isolated antibody, exemplified by antibody AA but also applicable to all antibodies disclosed herein, or an antigen binding fragment thereof, which specifically binds to sclerostin and wherein the variable domain of the heavy chain comprises at least one CDR having the sequences given in SEQ ID NO:101, 107, 113, 119, 125, 131, 137, 143, 149, 155, 161, 167, 173, 179, 185, 191, 197, 203, 209, 215, 221, 227, and 233 for CDR-H1; SEQ ID NO:102, 108, 114, 120, 132, 144, 150, 156, 162, 168, 174, 180, 186, 192, 198, 204, 210, 216, 222, 228, and 234 for CDR-H2; and SEQ ID NO:103, 109, 115, 121, 127, 133, 139, 145, 151, 157, 163, 169, 175, 181, 187, 193, 199, 205, 211, 217, 223, 229, and 235 for CDR-H3. The antibody or antigen binding fragment thereof may comprise a heavy chain variable domain in which the CDRs consist of at least one of the peptides of SEQ ID NO:101, 107, 113, 119, 125, 131, 137, 143, 149, 155, 161, 167, 173, 179, 185, 191, 197, 203, 209, 215, 221, 227, and 233 for CDR-H1; SEQ ID NO:102, 108, 114, 120, 132, 144, 150, 156, 162, 168, 174, 180, 186, 192, 198, 204, 210, 216, 222, 228, and 234 for CDR-H2; and SEQ ID NO: 103, 109, 115, 121, 127, 133, 139, 145, 151, 157, 163, 169, 175, 181, 187, 193, 199, 205, 211, 217, 223, 229, and 235 for CDR-H3.

When a light chain is present in antibodies of the invention the light chain may be any suitable complementary chain and may in particular be selected from a light chain wherein the variable domain comprises at least one or two or all of the CDRs consisting of (or comprising) at least one of the peptides of SEQ ID NO:98, 104, 110, 116, 122, 128, 134, 140, 146, 152, 158, 164, 170, 176, 182, 188, 194, 200, 206, 212, 218, 224, and 230 for CDR-L1; SEQ ID NO:99, 105, 111, 117, 123, 129, 135, 141, 147, 153, 159, 165, 171, 177, 183, 189, 195, 201, 207, 213, 219, 225, 231 for CDR-L2; and SEQ ID NO:100, 106, 112, 118, 124, 130, 136, 142, 148, 154, 160, 166, 172, 178, 184, 190, 196, 202, 208, 214, 220, 226, and 232 for CDR-L3.

Once synthesized, the DNA encoding an antibody of the invention or fragment thereof may be propagated and expressed according to any of a variety of well-known procedures for nucleic acid excision, ligation, transformation, and transfection using any number of known expression vectors. Thus, in certain embodiments, expression of an antibody fragment may be preferred in a prokaryotic host, such as Escherichia coli (see, e.g., Pluckthun et al., Methods Enzymol., 178:497-515 (1989). In certain other embodiments, expression of the antibody or a fragment thereof may be preferred in a eukaryotic host cell, including yeast (e.g., Saccharomyces cerevisiae, Schizosaccharomyces pombe, and Pichia pastoris), animal cells (including mammalian cells) or plant cells. Examples of suitable animal cells include, but are not limited to, myeloma (such as a mouse NSO line), COS, CHO, or hybridoma cells. Examples of plant cells include tobacco, corn, soybean, and rice cells.

One or more replicable expression vectors containing DNA encoding an antibody variable and/or constant region may be prepared and used to transform an appropriate cell line, for example, a non-producing myeloma cell line, such as a mouse NSO line or a bacteria, such as E. coli, in which production of the antibody will occur. In order to obtain efficient transcription and translation, the DNA sequence in each vector should include appropriate regulatory sequences, particularly a promoter and leader sequence operatively linked to the variable domain sequence. Particular methods for producing antibodies in this way are generally well-known and routinely used. For example, basic molecular biology procedures are described by Maniatis et al., Molecular Cloning, A Laboratory Manual, 2d ed., Cold Spring Harbor Laboratory, New York, 1989. See also Maniatis et al, 3d ed., Cold Spring Harbor Laboratory, New York, (2001). DNA sequencing can be performed as described in Sanger et al., PNAS, 74:5463 (1977), and the Amersham International plc sequencing handbook, and site directed mutagenesis can be carried out according to methods known in the art (Kramer et al., Nucleic Acids Res., 12:9441 (1984); Kunkel, Proc. Natl. Acad. Sci. USA, 82:488-92 (1985); Kunkel et al., Methods in Enzymol., 154:367-82 (1987); the Anglian Biotechnology Ltd handbook). Additionally, numerous publications describe techniques suitable for the preparation of antibodies by manipulation of DNA, creation of expression vectors, and transformation and culture of appropriate cells (Mountain, A. and Adair, J. R., in Biotechnology and Genetic Engineering Reviews, (Tombs, M. P. (ed.), 10, Chapter 1, Intercept, Andover, UK (1992)); Current Protocols in Molecular Biology, F. M. Ausubel (ed.), Wiley Interscience, New York (1999)).

Antibodies with improved affinities containing one or more of the above-mentioned CDRs can be obtained by a number of affinity maturation protocols including maintaining the CDRs (Yang et al., J. Mol. Biol., 254:392-403 (1995), chain shuffling (Marks et al., Bio/Technology, 10:779-783 (1992), use of mutation strains of E. coli. (Low et al., J. Mol. Biol., 250:350-368 (1996), DNA shuffling (Patten et al., Curr. Opin. Biotechnol., 8:724-733 (1997)), phage display (Thompson et al., J. Mol. Biol., 256:7-88 (1996)) and sexual PCR (Crameri, et al., Nature, 391:288-291 (1998)). All of these methods of affinity maturation are discussed by Vaughan et al., (Nature Biotechnology, 16:535-539 (1998)).

Other antibodies according to the invention may be obtained by conventional immunization and cell fusion procedures as described herein and known in the art. Monoclonal antibodies of the invention may be generated using a variety of known techniques. In general, monoclonal antibodies that bind to specific antigens may be obtained by methods known to those skilled in the art (see, for example, Kohler et al., Nature, 256:495 (1975); Coligan et al. (eds.), Current Protocols in Immunology, 1:2.5.12.6.7 (John Wiley & Sons 1991); U.S. Pat. Nos. RE 32,011, 4,902,614, 4,543,439, and 4,411,993; Monoclonal Antibodies, Hybridomas: A New Dimension in Biological Analyses, Plenum Press, Kennett, McKearn, and Bechtol (eds.) (1980); and Antibodies: A Laboratory Manual, Harlow and Lane (eds.), Cold Spring Harbor Laboratory Press (1988); Picksley et al., “Production of monoclonal antibodies against proteins expressed in E. coli,” in DNA Cloning 2: Expression Systems, 2d Ed., Glover et al. (eds.), page 93 (Oxford University Press 1995)). Antibody fragments may be derived therefrom using any suitable standard technique such as proteolytic digestion, or optionally, by proteolytic digestion (for example, using papain or pepsin) followed by mild reduction of disulfide bonds and alkylation. Alternatively, such fragments may also be generated by recombinant genetic engineering techniques as described herein.

Monoclonal antibodies can be obtained by injecting an animal, for example, a rat, hamster, a rabbit, or preferably a mouse, including for example a transgenic or a knock-out, as known in the art, with an immunogen comprising human sclerostin of SEQ ID NO:1, or a fragment thereof, according to methods known in the art and described herein. Specific antibody production may be monitored after the initial injection and/or after a booster injection by obtaining a serum sample and detecting the presence of an antibody that binds to human sclerostin (or fragment thereof) using any one of several immunodetection methods known in the art and described herein. From animals producing the desired antibodies, lymphoid cells, most commonly cells from the spleen or lymph node, are removed to obtain B-lymphocytes. The B-lymphocytes are then fused with a drug-sensitized myeloma cell fusion partner, preferably one that is syngeneic with the immunized animal and that optionally has other desirable properties (e.g., inability to express endogenous Ig gene products, e.g., P3×63-Ag 8.653 (ATCC No. CRL 1580); NSO, SP20) to produce hybridomas, which are immortal eukaryotic cell lines. The lymphoid (e.g., spleen) cells and the myeloma cells may be combined for a few minutes with a membrane fusion-promoting agent, such as polyethylene glycol or a nonionic detergent, and then plated at low density on a selective medium that supports the growth of hybridoma cells but not unfused myeloma cells. A preferred selection media is HAT (hypoxanthine, aminopterin, thymidine). After a sufficient time, usually about one to two weeks, colonies of cells are observed. Single colonies are isolated, and antibodies produced by the cells may be tested for binding activity to human sclerostin, using any one of a variety of immunoassays known in the art and described herein. The hybridomas are cloned (e.g., by limited dilution cloning or by soft agar plaque isolation) and positive clones that produce an antibody specific to sclerostin are selected and cultured. The monoclonal antibodies from the hybridoma cultures may be isolated from the supernatants of hybridoma cultures. An alternative method for production of a murine monoclonal antibody is to inject the hybridoma cells into the peritoneal cavity of a syngeneic mouse, for example, a mouse that has been treated (e.g., pristane-primed) to promote formation of ascites fluid containing the monoclonal antibody. Monoclonal antibodies can be isolated and purified by a variety of well-established techniques. Such isolation techniques include affinity chromatography with Protein-A Sepharose, size-exclusion chromatography, and ion-exchange chromatography (see, for example, Coligan at pages 2.7.1-2.7.12 and pages 2.9.1-2.9.3; Baines et al., “Purification of Immunoglobulin G (IgG),” in Methods in Molecular Biology, Vol. 10, pages 79-104, Humana Press, Inc. (1992)). Monoclonal antibodies may be purified by affinity chromatography using an appropriate ligand selected based on particular properties of the antibody (e.g., heavy or light chain isotype, binding specificity, etc.). Examples of a suitable ligand, immobilized on a solid support, include Protein A, Protein G, an anticonstant region (light chain or heavy chain) antibody, an anti-idiotype antibody, and a TGF-beta binding protein, or fragment or variant thereof.

An antibody of the present invention may also be a human monoclonal antibody. Human monoclonal antibodies may be generated by any number of techniques with which those having ordinary skill in the art will be familiar. Such methods include, but are not limited to, Epstein Barr Virus (EBV) transformation of human peripheral blood cells (e.g., containing B lymphocytes), in vitro immunization of human B cells, fusion of spleen cells from immunized transgenic mice carrying inserted human immunoglobulin genes, isolation from human immunoglobulin V region phage libraries, or other procedures as known in the art and based on the disclosure herein. For example, human monoclonal antibodies may be obtained from transgenic mice that have been engineered to produce specific human antibodies in response to antigenic challenge. Methods for obtaining human antibodies from transgenic mice are described, for example, by Green et al., Nature Genet., 7:13 (1994); Lonberg et al., Nature, 368:856 (1994); Taylor et al., Int. Immun., 6:579 (1994); U.S. Pat. No. 5,877,397; Bruggemann et al., Curr. Opin. Biotechnol., 8:455-58 (1997); Jakobovits et al., Ann. N.Y. Acad. Sci., 764:525-35 (1995). In this technique, elements of the human heavy and light chain locus are introduced into strains of mice derived from embryonic stem cell lines that contain targeted disruptions of the endogenous heavy chain and light chain loci (see also Bruggemann et al., Curr. Opin. Biotechnol., 8:455-58 (1997)). For example, human immunoglobulin transgenes may be mini-gene constructs, or transloci on yeast artificial chromosomes, which undergo B cell-specific DNA rearrangement and hypermutation in the mouse lymphoid tissue. Human monoclonal antibodies may be obtained by immunizing the transgenic mice, which may then produce human antibodies specific for sclerostin. Lymphoid cells of the immunized transgenic mice can be used to produce human antibody-secreting hybridomas according to the methods described herein. Polyclonal sera containing human antibodies may also be obtained from the blood of the immunized animals.

Another method for generating human antibodies of the invention includes immortalizing human peripheral blood cells by EBV transformation. See, e.g., U.S. Pat. No. 4,464,456. Such an immortalized B cell line (or lymphoblastoid cell line) producing a monoclonal antibody that specifically binds to sclerostin can be identified by immunodetection methods as provided herein, for example, an ELISA, and then isolated by standard cloning techniques. The stability of the lymphoblastoid cell line producing an anti-sclerostin antibody may be improved by fusing the transformed cell line with a murine myeloma to produce a mouse-human hybrid cell line according to methods known in the art (see, e.g., Glasky et al., Hybridoma 8:377-89 (1989)). Still another method to generate human monoclonal antibodies is in vitro immunization, which includes priming human splenic B cells with human sclerostin, followed by fusion of primed B cells with a heterohybrid fusion partner. See, e.g., Boerner et al., J. Immunol., 147:86-95 (1991).

In certain embodiments, a B cell that is producing an anti-human sclerostin antibody is selected and the light chain and heavy chain variable regions are cloned from the B cell according to molecular biology techniques known in the art (WO 92/02551; U.S. Pat. No. 5,627,052; Babcook et al., Proc. Natl. Acad. Sci. USA, 93:7843-48 (1996)) and described herein. B cells from an immunized animal may be isolated from the spleen, lymph node, or peripheral blood sample by selecting a cell that is producing an antibody that specifically binds to sclerostin. B cells may also be isolated from humans, for example, from a peripheral blood sample. Methods for detecting single B cells that are producing an antibody with the desired specificity are well known in the art, for example, by plaque formation, fluorescence-activated cell sorting, in vitro stimulation followed by detection of specific antibody, and the like. Methods for selecting specific antibody-producing B cells include, for example, preparing a single cell suspension of B cells in soft agar that contains human sclerostin. Binding of the specific antibody produced by the B cell to the antigen results in the formation of a complex, which may be visible as an immunoprecipitate. After the B cells producing the desired antibody are selected, the specific antibody genes may be cloned by isolating and amplifying DNA or mRNA according to methods known in the art and described herein.

An additional method for obtaining antibodies of the invention is by phage display. See, e.g., Winter et al., Annu. Rev. Immunol., 12:433-55 (1994); Burton et al., Adv. Immunol., 57:191-280 (1994). Human or murine immunoglobulin variable region gene combinatorial libraries may be created in phage vectors that can be screened to select Ig fragments (Fab, Fv, sFv, or multimers thereof) that bind specifically to TGF-beta binding protein or variant or fragment thereof. See, e.g., U.S. Pat. No. 5,223,409; Huse et al., Science, 246:1275-81 (1989); Sastry et al., Proc. Natl. Acad. Sci. USA, 86:5728-32 (1989); Alting-Mees et al., Strategies in Molecular Biology, 3:1-9 (1990); Kang et al., Proc. Natl. Acad. Sci. USA, 88:4363-66 (1991); Hoogenboom et al., J. Molec. Biol., 227:381-388 (1992); Schlebusch et al., Hybridoma, 16:47-52 (1997) and references cited therein. For example, a library containing a plurality of polynucleotide sequences encoding Ig variable region fragments may be inserted into the genome of a filamentous bacteriophage, such as M13 or a variant thereof, in frame with the sequence encoding a phage coat protein. A fusion protein may be a fusion of the coat protein with the light chain variable region domain and/or with the heavy chain variable region domain. According to certain embodiments, immunoglobulin Fab fragments may also be displayed on a phage particle (see, e.g., U.S. Pat. No. 5,698,426).

Heavy and light chain immunoglobulin cDNA expression libraries may also be prepared in lambda phage, for example, using λImmunoZap™(H) and λImmunoZap™(L) vectors (Stratagene, La Jolla, Calif.). Briefly, mRNA is isolated from a B cell population, and used to create heavy and light chain immunoglobulin cDNA expression libraries in the λImmunoZap(H) and λImmunoZap(L) vectors. These vectors may be screened individually or co-expressed to form Fab fragments or antibodies (see Huse et al., supra; see also Sastry et al., supra). Positive plaques may subsequently be converted to a non-lytic plasmid that allows high level expression of monoclonal antibody fragments from E. coli.

In one embodiment, in a hybridoma the variable regions of a gene expressing a monoclonal antibody of interest are amplified using nucleotide primers. These primers may be synthesized by one of ordinary skill in the art, or may be purchased from commercially available sources. See, e.g., Stratagene (La Jolla, Calif.), which sells primers for mouse and human variable regions including, among others, primers for V_{Ha}, V_{Hb}, V_{Hc}, V_{Hd}, C_{H1}, V_{L} and CL regions. These primers may be used to amplify heavy or light chain variable regions, which may then be inserted into vectors such as ImmunoZAP™H or ImmunoZAP™L (Stratagene), respectively. These vectors may then be introduced into E. coli, yeast, or mammalian-based systems for expression. Large amounts of a single-chain protein containing a fusion of the V_{H} and V_{L} domains may be produced using these methods (see Bird et al., Science, 242:423-426, (1988)).

Once cells producing antibodies according to the invention have been obtained using any of the above-described immunization and other techniques, the specific antibody genes may be cloned by isolating and amplifying DNA or mRNA therefrom according to standard procedures as described herein. The antibodies produced therefrom may be sequenced and the CDRs identified and the DNA coding for the CDRs may be manipulated as described previously to generate other antibodies according to the invention.

Preferably the binding agents specifically bind to sclerostin. As with all binding agents and binding assays, one of skill in this art recognizes that the various moieties to which a binding agent should not detectably bind in order to be therapeutically effective and suitable would be exhaustive and impractical to list. Therefore, for a binding agent disclosed herein, the term “specifically binds” refers to the ability of a binding agent to bind to sclerostin, preferably human sclerostin, with greater affinity than it binds to an unrelated control protein. Preferably the control protein is hen egg white lysozyme. Preferably the binding agents bind to sclerostin with an affinity that is at least, 50, 100, 250, 500, 1000, or 10,000 times greater than the affinity for a control protein. A binding agent may have a binding affinity for human sclerostin of less than or equal to 1×10⁻⁷ M, less than or equal to 1×10⁻⁸ M, less than or equal to 1×10⁻⁹ M, less than or equal to 1×10⁻¹⁰ M, less than or equal to 1×10⁻¹¹ M, or less than or equal to 1×10⁻¹² M.

Affinity may be determined by an affinity ELISA assay. In certain embodiments, affinity may be determined by a BIAcore assay. In certain embodiments, affinity may be determined by a kinetic method. In certain embodiments, affinity may be determined by an equilibrium/solution method. Such methods are described in further detail herein or known in the art.

Sclerostin binding agents of the present invention preferably modulate sclerostin function in the cell-based assay described herein and/or the in vivo assay described herein and/or bind to one or more of the epitopes described herein and/or cross-block the binding of one of the antibodies described in this application and/or are cross-blocked from binding sclerostin by one of the antibodies described in this application. Accordingly such binding agents can be identified using the assays described herein.

In certain embodiments, binding agents are generated by first identifying antibodies that bind to one more of the epitopes provided herein and/or neutralize in the cell-based and/or in vivo assays described herein and/or cross-block the antibodies described in this application and/or are cross-blocked from binding sclerostin by one of the antibodies described in this application. The CDR regions from these antibodies are then used to insert into appropriate biocompatible frameworks to generate sclerostin binding agents. The non-CDR portion of the binding agent may be composed of amino acids, or may be a non-protein molecule. The assays described herein allow the characterization of binding agents. Preferably the binding agents of the present invention are antibodies as defined herein.

It will be understood by one skilled in the art that some proteins, such as antibodies, may undergo a variety of posttranslational modifications. The type and extent of these modifications often depends on the host cell line used to express the protein as well as the culture conditions. Such modifications may include variations in glycosylation, methionine oxidation, diketopiperazine formation, aspartate isomerization and asparagine deamidation. A frequent modification is the loss of a carboxy-terminal basic residue (such as lysine or arginine) due to the action of carboxypeptidases (as described in Harris, R. J., Journal of Chromatography, 705:129-134 (1995)).

The Kappa Constant region for all VK regions of antibodies AA-CC, EE-JJ, and LL-WW disclosed herein is as follows:

(SEQ ID NO: 2)

TDAAPTVSIFPPSSEQLTSGGASVVCFLNNFYPKDINVKWKIDGSERQNG
VLNSWTDQDSKDSTYSMSSTLTLTKDEYERHNSYTCEATHKTSTSPIVKS
FNRNEC

The Heavy Constant Region for all V_{H} regions of antibodies AA-WW in this Example is as follows:

(SEQ ID NO: 3)

AKTTPPSVYPLAPGSAAQTNSMVTLGCLVKGYFPEPVTVTWNSGSLSSGV
HTFPAVLQSDLYTLSSSVTVPSSTWPSETVTCNVAHPASSTKVDKKIVPR
DCGCKPCICTVPEVSSVFIFPPKPKDVLTITLTPKVTCVVVDISKDDPEV
QFSWFVDDVEVHTAQTQPREEQFNSTFRSVSELPIMHQDWLNGKEFKCRV
NSAAFPAPIEKTISKTKGRPKAPQVYTIPPPKEQMAKDKVSLTCMITDFF
PEDITVEWQWNGQPAENYKNTQPIMDTDGSYFVYSKLNVQKSNWEAGNTF
TCSVLHEGLHNHHTEKSLSHSPGK

In the following antibody amino acid sequences, the boxed-shaded amino acids represent complement-determining regions (CDRs) and the underlined amino acids represent signal peptide.

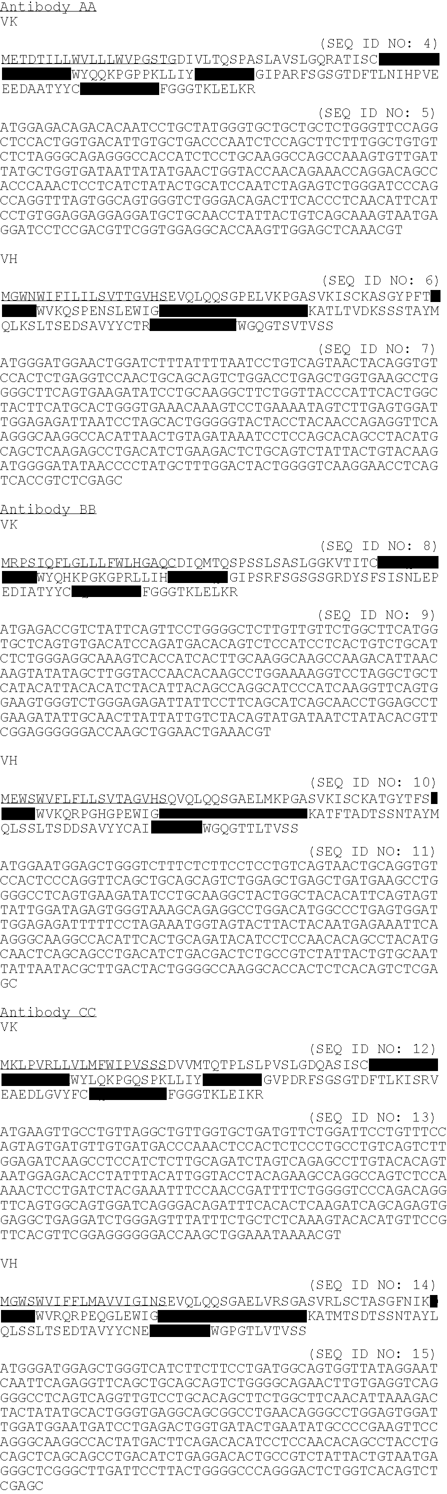

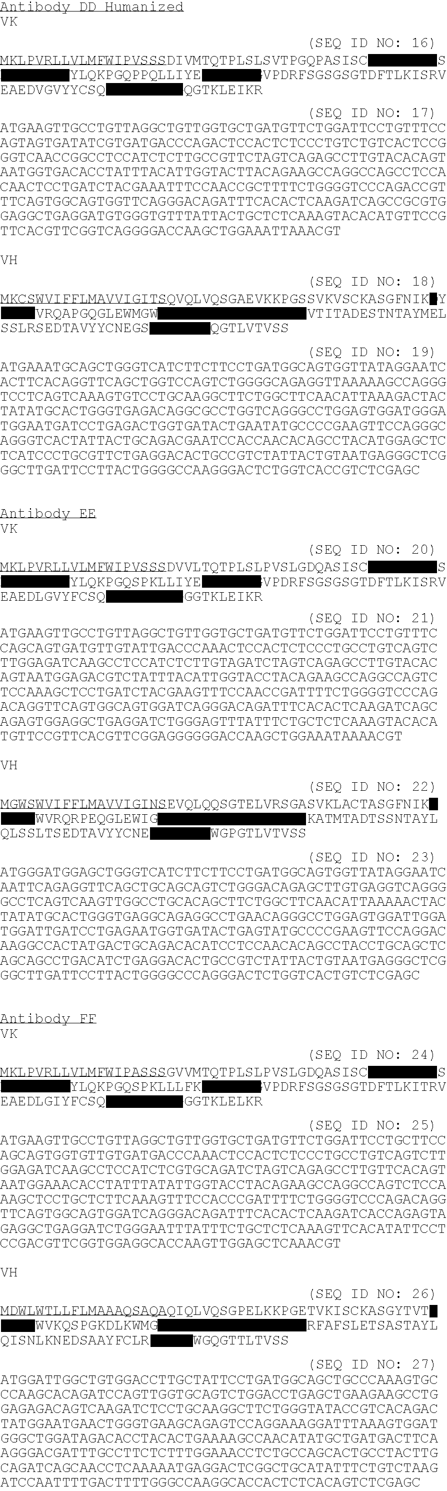

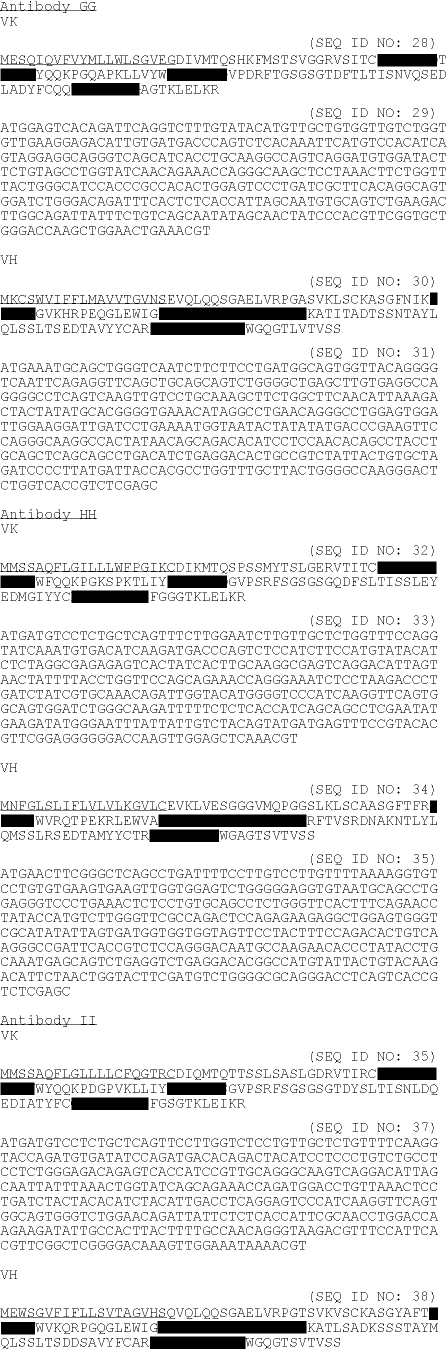

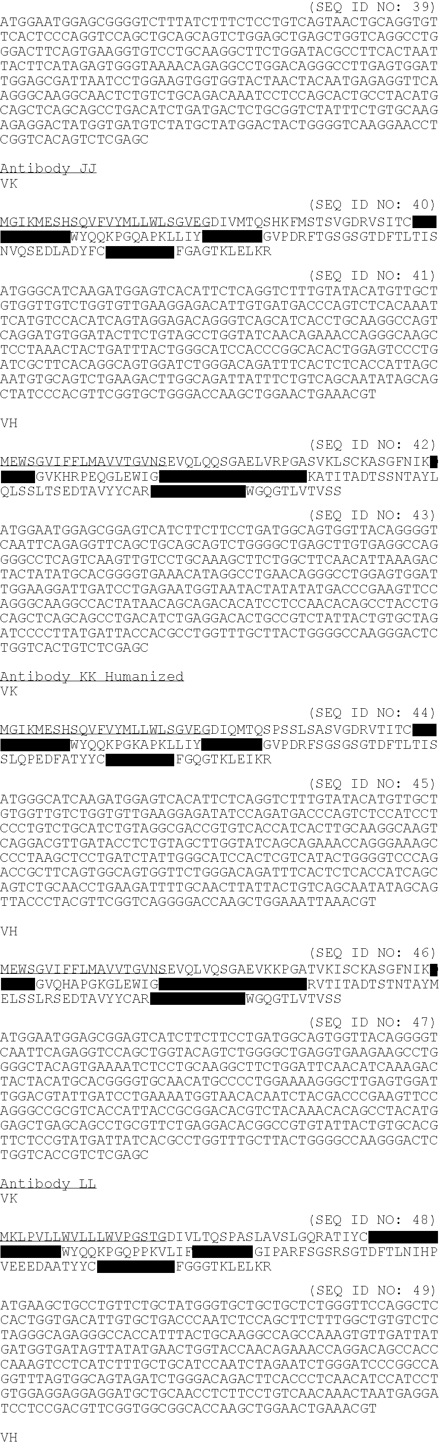

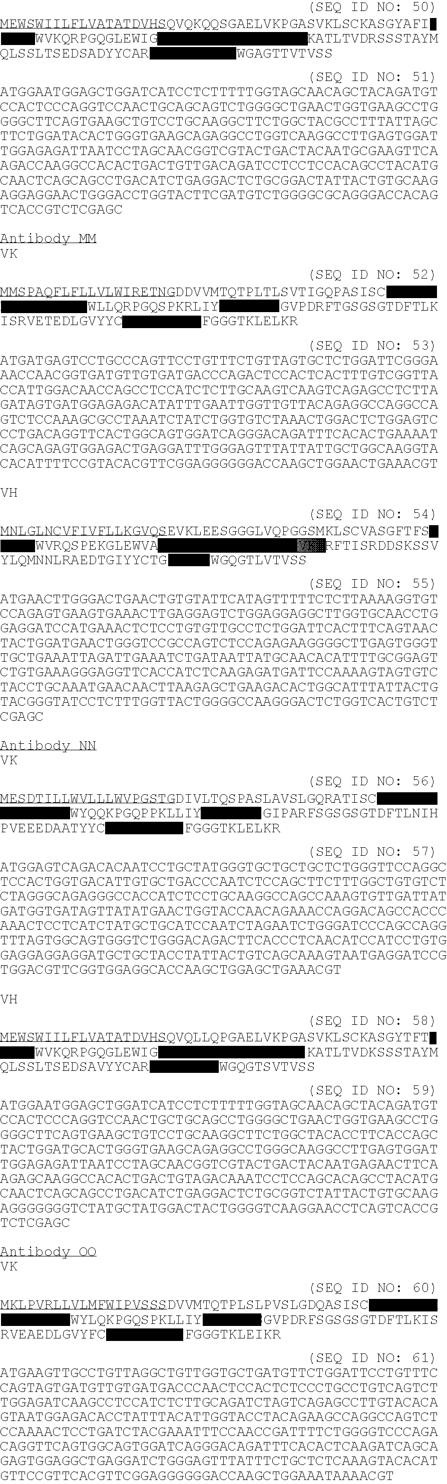

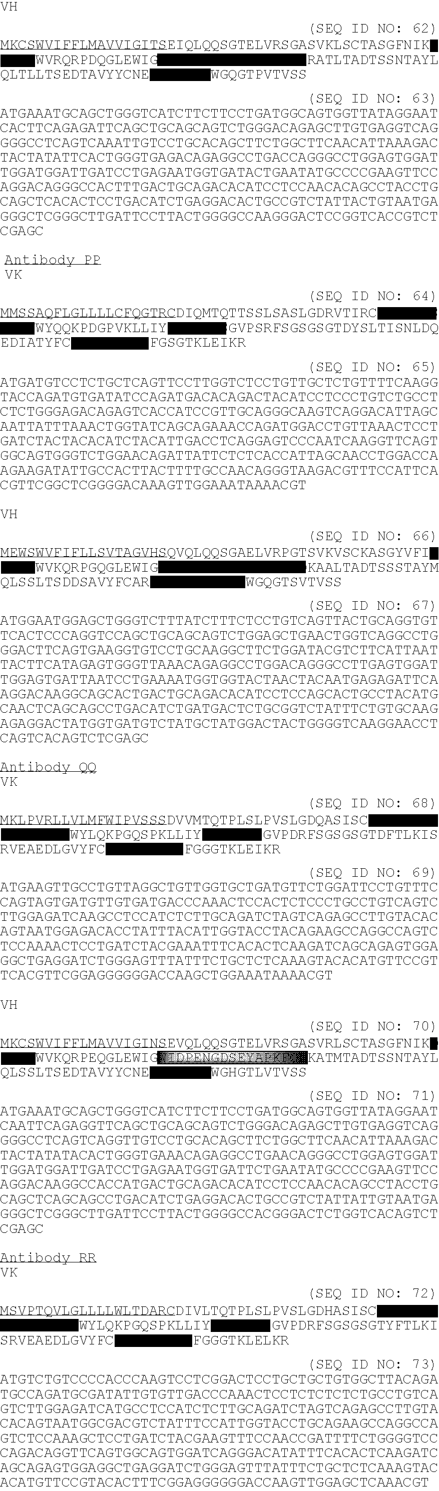

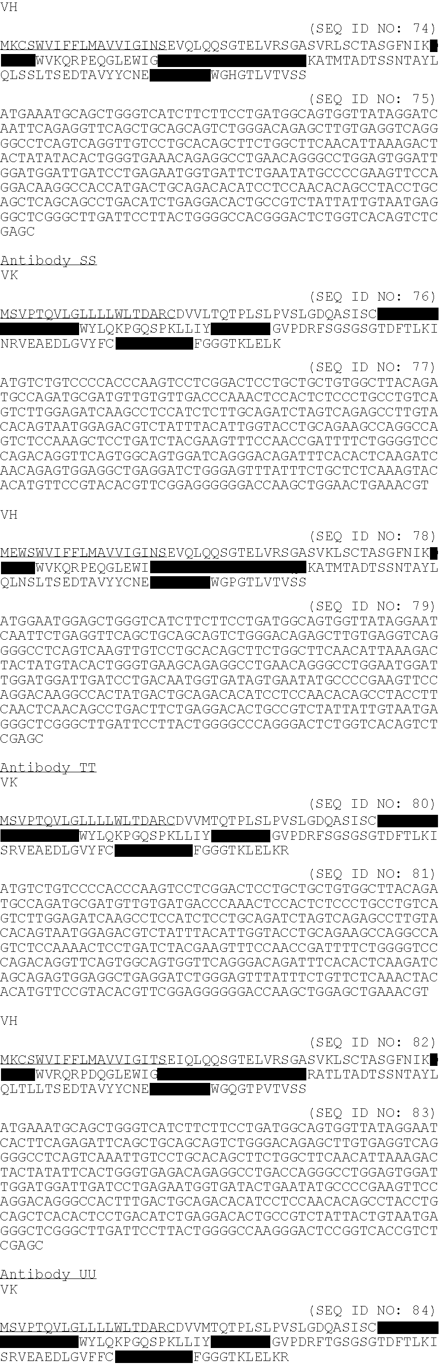

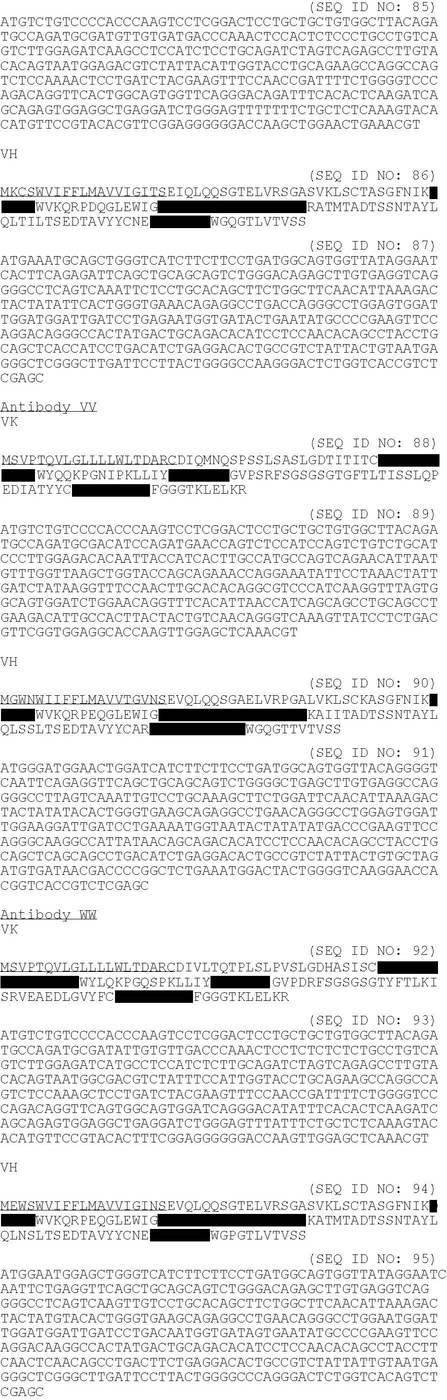

For humanized antibodies DD and KK, light chain human kappa constant regions are:

(SEQ ID NO: 96)

TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC*

and heavy chain human gamma-4 constant regions are:

(SEQ ID NO: 97)

ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK*

The hinge region contains the Ser-241-Pro mutation to improve hinge stability (Angal S et al, Mol. Immunol, 30(1):105-108 (1993)).

The following table, Table 1, provides the SEQ ID NOs for the antibody polynucleotides and polypeptides disclosed above, without leader sequences.

TABLE 1

| | POLYPEPTIDES | | POLYNUCLEOTIDES | |
|---|---|---|---|---|
| | SEQ ID NO | SEQ ID NO | SEQ ID NO | SEQ ID NO |
| Antibody | with leader | without leader | with leader | without leader |
| AA VK | 4 | 236 | 5 | 237 |
| AA VH | 6 | 238 | 7 | 239 |
| BB VK | 8 | 240 | 9 | 241 |
| BB VH | 10 | 242 | 11 | 243 |
| CC VK | 12 | 244 | 13 | 245 |
| CC VH | 14 | 246 | 15 | 247 |
| DD VK | 16 | 248 | 17 | 249 |
| DD VH | 18 | 250 | 19 | 251 |
| EE VK | 20 | 252 | 21 | 253 |
| EE VH | 22 | 254 | 23 | 255 |
| FF VK | 24 | 256 | 25 | 257 |
| FF VH | 26 | 258 | 27 | 259 |
| GG VK | 28 | 260 | 29 | 261 |
| GG VH | 30 | 262 | 31 | 263 |
| HH VK | 32 | 264 | 33 | 265 |
| HH VH | 34 | 266 | 35 | 267 |
| II VK | 36 | 268 | 37 | 269 |
| II VH | 38 | 270 | 39 | 271 |
| JJ VK | 40 | 272 | 41 | 273 |
| JJ VH | 42 | 274 | 43 | 275 |
| KK VK | 44 | 276 | 45 | 277 |
| KK VH | 46 | 278 | 47 | 279 |
| LL VK | 48 | 280 | 49 | 281 |
| LL VH | 50 | 282 | 51 | 283 |
| MM VK | 52 | 284 | 53 | 285 |
| MM VH | 54 | 286 | 55 | 287 |
| NN VK | 56 | 288 | 57 | 289 |
| NN VH | 58 | 290 | 59 | 291 |
| OO VK | 60 | 292 | 61 | 293 |
| OO VH | 62 | 294 | 63 | 295 |
| PP VK | 64 | 296 | 65 | 297 |
| PP VH | 66 | 298 | 67 | 299 |
| QQ VK | 68 | 300 | 69 | 301 |
| QQ VH | 70 | 302 | 71 | 303 |
| RR VK | 72 | 304 | 73 | 305 |
| RR VH | 74 | 306 | 75 | 307 |
| SS VK | 76 | 308 | 77 | 309 |
| SS VH | 78 | 310 | 79 | 311 |
| TT VK | 80 | 312 | 81 | 313 |
| TT VH | 82 | 314 | 83 | 315 |
| UU VK | 84 | 316 | 85 | 317 |
| UU VH | 86 | 318 | 87 | 319 |
| VV VK | 88 | 320 | 89 | 321 |
| VV VH | 90 | 322 | 91 | 323 |
| WW VK | 92 | 324 | 93 | 325 |
| WW VH | 94 | 326 | 95 | 327 |

Table 2 below provides the SEQ ID NOs and amino acid sequences of the CDR's of AA-WW. L1, L2, and L3 refer to light chain CDR's 1, 2, and 3, and H1, H2, and H3 refer to heavy chain CDR's 1, 2, and 3 according to the Kabat numbering system (Kabat et al., 1987 in Sequences of Proteins of Immunological Interest, U.S. Department of Health and Human Services, NIH, USA).

TABLE 2

| | | | |
|---|---|---|---|
| Antibody | CDR 1 | CDR 2 | CDR 3 |

| | | | |
|---|---|---|---|
| AA VK | KASQSVDYAGDNYMN | TASNLES | QQSNEDPPT |
| | (SEQ ID NO: 98) | (SEQ ID NO: 99) | (SEQ ID NO: 100) |
| AA VH | GYFMH | EINPSTGGTTYNQRFKG | WGYNPYALDY |
| | (SEQ ID NO: 101) | (SEQ ID NO: 102) | (SEQ ID NO: 103) |
| BB VK | KASQDINKYIA | YTSTLQP | LQYDNLYT |
| | (SEQ ID NO: 104) | (SEQ ID NO: 105) | (SEQ ID NO: 106) |
| BB VH | SYWIE | EIFPRNGSTYYNEKFKG | INTLDY |
| | (SEQ ID NO: 107) | (SEQ ID NO: 108) | (SEQ ID NO: 109) |
| CC VK | RSSQSLVHSNGDTYLH | EISNRFS | SQSTHVPFT |
| | (SEQ ID NO: 110) | (SEQ ID NO: 111) | (SEQ ID NO: 112) |
| CC VH | DYYMH | WNDPETGDTEYAPKFQG | GSGLIPY |
| | (SEQ ID NO: 113) | (SEQ ID NO: 114) | (SEQ ID NO: 115) |
| DD VK | RSSQSLVHSNGDTYLH | EISNRFS | SQSTHVPFT |
| | (SEQ ID NO: 116) | (SEQ ID NO: 117) | (SEQ ID NO: 118) |
| DD VH | DYYMH | WNDPETGDTEYAPKFQG | GSGLIPY |
| | (SEQ ID NO: 119) | (SEQ ID NO: 120) | (SEQ ID NO: 121) |
| EE VK | RSSQSLVHSNGDVYLH | EVSNRFS | SQSTHVPFT |
| | (SEQ ID NO: 122) | (SEQ ID NO: 123) | (SEQ ID NO: 124) |
| EE VH | NYYMH | WNDPETGDTEYAPKFQG | GSGLIPY |
| | (SEQ ID NO: 125) | (SEQ ID NO: 126) | (SEQ ID NO: 127) |
| FF K | RSSQSLVHSNGNTYLY | KVSTRFS | SQSSHIPPT |
| | (SEQ ID NO: 128) | (SEQ ID NO: 129) | (SEQ ID NO: 130) |
| FF VH | DYGMN | WIDTYTEKPTYADDFKG | SNFDF |
| | (SEQ ID NO: 131) | (SEQ ID NO: 132) | (SEQ ID NO: 133) |
| GG VK | KASQDVDTSVA | WASTRHT | QQYSNYPT |
| | (SEQ ID NO: 134) | (SEQ ID NO: 135) | (SEQ ID NO: 136) |
| GG VH | DYYMH | RTDPENGNTIYDPKFQG | SPYDYHAWFAY |
| | (SEQ ID NO: 137) | (SEQ ID NO: 138) | (SEQ ID NO: 139) |
| HH VK | KASQDISNYFT | RANRLVD | LQYDEFPYT |
| | (SEQ ID NO: 140) | (SEQ ID NO: 141) | (SEQ ID NO: 142) |
| HH VH | TYTMS | YISDGGGSSYFPDTVKG | HSNWYFDV |
| | (SEQ ID NO: 143) | (SEQ ID NO: 144) | (SEQ ID NO: 145) |
| II VK | RASQDISNYLN | YTSTLTS | QQGKTFPFT |
| | (SEQ ID NO: 146) | (SEQ ID NO: 147) | (SEQ ID NO: 148) |
| II VH | NYFIE | AINPGSGGTNYNERFKG | EDYGDVYAMDY |
| | (SEQ ID NO: 149) | (SEQ ID NO: 150) | (SEQ ID NO: 151) |
| JJ VK | KASQDVDTSVA | WASTRHT | QQYSSYPT |
| | (SEQ ID NO: 152) | (SEQ ID NO: 153) | (SEQ ID NO: 154) |
| JJ VH | DYYMH | RIDPENGNTIYDPKFQG | SPYDYHAWFAY |
| | (SEQ ID NO: 155) | (SEQ ID NO: 156) | (SEQ ID NO: 157) |
| KK VK | KASQDVDTSVA | WASTRHT | QQYSSYPT |
| | (SEQ ID NO: 158) | (SEQ ID NO: 159) | (SEQ ID NO: 160) |
| KK VH | DYYMH | RIDPENGNTIYDPKFQG | SPYDYHAWFAY |
| | (SEQ ID NO: 161) | (SEQ ID NO: 162) | (SEQ ID NO: 163) |
| LL VK | KASQSVDYDGDSYMN | AASNLES | QQTNEDPPT |
| | (SEQ ID NO: 164) | (SEQ ID NO: 165) | (SEQ ID NO: 166) |
| LL VH | SFWIH | EINPSNGRTDYNAKFKT | GGTGTWYFDV |
| | (SEQ ID NO: 167) | (SEQ ID NO: 168) | (SEQ ID NO: 169) |
| MM VK | KSSQSLLDSDGETYLN | LVSKLDS | WQGTHFPYT |
| | (SEQ ID NO: 170) | (SEQ ID NO: 171) | (SEQ ID NO: 172) |
| MM VH | NYWMN | EIRLKSDNYATHFAESVK | ILFGY |
| | (SEQ ID NO: 173) | G | (SEQ ID NO: 175) |
| | | (SEQ ID NO: 174) | |
| NN VK | KASQSVDYDGDSYMN | AASNLES | QQSNEDPWT |
| | (SEQ ID NO: 176) | (SEQ ID NO: 177) | (SEQ ID NO: 178) |
| NN VH | SYWMH | EINPSNGRTDYNENFKS | GGVYAMDY |
| | (SEQ ID NO: 179) | (SEQ ID NO: 180) | (SEQ ID NO: 181) |
| OO VK | RSSQSLVHSNGDTYLH | EISNRFS | SQSTHVPFT |
| | (SEQ ID NO: 182) | (SEQ ID NO: 183) | (SEQ ID NO: 184) |
| OO VH | DYYIH | WIDPENGDTEYAPKFQD | GSGLIPY |
| | (SEQ ID NO: 185) | (SEQ ID NO: 186) | (SEQ ID NO: 187) |
| PP VK | RASQDISNYLN | YTSTLTS | QQGKTFPFT |
| | (SEQ ID NO: 188) | (SEQ ID NO: 189) | (SEQ ID NO: 190) |
| PP VH | NYFIE | VINPENGGTNYNERFKD | EDYGDVYAMDY |
| | (SEQ ID NO: 191) | (SEQ ID NO: 192) | (SEQ ID NO: 193) |
| QQ VK | RSSQSLVHSNGDTYLH | EISNRFS | SQSTHVPFT |
| | (SEQ ID NO: 194) | (SEQ ID NO: 195) | (SEQ ID NO: 196) |
| QQ VH | DYYIH | WIDPENGDSEYAPKFQD | GSGLIPY |
| | (SEQ ID NO: 197) | (SEQ ID NO: 198) | (SEQ ID NO: 199) |
| RR VK | RSSQSLVHSNGDVYFH | EVSNRFS | SQSTHVPYT |
| | (SEQ ID NO: 200) | (SEQ ID NO: 201) | (SEQ ID NO: 202) |
| RR VH | DYYIH | WIDPENGDSEYAPKFQD | GSGLIPY |
| | (SEQ ID NO: 203) | (SEQ ID NO: 204) | (SEQ ID NO: 205) |
| SS VK | RSSQSLVHSNGDVYFH | EVSNRFS | SQSTHVPYT |
| | (SEQ ID NO: 206) | (SEQ ID NO: 207) | (SEQ ID NO: 208) |
| SS VH | DYYVH | WIDPDNGDSEYAPKFQD | GSGLIPY |
| | (SEQ ID NO: 209) | (SEQ ID NO: 210) | (SEQ ID NO: 211) |
| TT VK | RSSQSLVHSNGDVYLH | EVSNRFS | SQTTHVPYT |
| | (SEQ ID NO: 212) | (SEQ ID NO: 213) | (SEQ ID NO: 214) |
| TT VH | DYYIH | WIDPENGDTEYAPKFQD | GSGLIPY |
| | (SEQ ID NO: 215) | (SEQ ID NO: 216) | (SEQ ID NO: 217) |
| UU VK | RSSQSLVHSNGDVYLH | EVSNRFS | SQSTHVPYT |
| | (SEQ ID NO: 218) | (SEQ ID NO: 219) | (SEQ ID NO: 220) |
| UU VH | DYYIH | WIDPENGDTEYAPKFQD | GSGLIPY |
| | (SEQ ID NO: 221) | (SEQ ID NO: 222) | (SEQ ID NO: 223) |
| VV VK | HASQNINVWLS | KVSNLHT | QQGQSYPLT |
| | (SEQ ID NO: 224) | (SEQ ID NO: 225) | (SEQ ID NO: 226) |
| VV VH | DYYIH | RIDPENGNTIYDPKFQG | CDNDPGSEMDY |
| | (SEQ ID NO: 227) | (SEQ ID NO: 228) | (SEQ ID NO: 229) |
| WW VK | RSSQSLVHSNGDVYFH | EVSNRFS | SQSTHVPYT |
| | (SEQ ID NO: 230) | (SEQ ID NO: 231) | (SEQ ID NO: 232) |
| WW VH | DYYVH | WIDPDNGDSEYAPKFQD | GSGLIPY |
| | (SEQ ID NO: 233) | (SEQ ID NO: 234) | (SEQ ID NO: 235) |

An oligopeptide or polypeptide is within the scope of the invention if it has an amino acid sequence that is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to least one of the CDR's of Table 2 above; and/or to a CDR of a sclerostin binding agent that cross-blocks the binding of at least one of antibodies AA-WW to sclerostin; and/or is cross-blocked from binding to sclerostin by at least one of antibodies and/or to a CDR of a sclerostin binding agent wherein the binding agent can block the inhibitory effect of sclerostin in a cell based mineralization assay (i.e., a sclerostin neutralizing binding agent).

Sclerostin binding agent polypeptides and antibodies are within the scope of the invention if they have amino acid sequences that are at least 85%, 86%, 87%, 88%, 89%, 90%, 91%, 92%, 93%, 94%, 95%, 96%, 97%, 98% or 99% identical to a variable region of at least one of antibodies AA-WW and cross-block the binding of at least one of antibodies AA-WW to sclerostin; and/or are cross-blocked from binding to sclerostin by at least one of antibodies AA-WW; and/or can block the inhibitory effect of sclerostin in a cell based mineralization assay (i.e., a sclerostin neutralizing binding agent).

Polynucleotides encoding sclerostin binding agents are within the scope of the invention if they have polynucleotide sequences that are at least 85%, 86%, 87%, 88%, 89%, 90%, 91%, 92%, 93%, 94%, 95%, 96%, 97%, 98% or 99% identical to a polynucleotide encoding a variable region of at least one of antibodies, and wherein the encoded sclerostin binding agents cross-block the binding of at least one of antibodies to sclerostin; and/or are cross-blocked from binding to sclerostin by at least one of antibodies AA-WW; and/or can block the inhibitory effect of sclerostin in a cell based mineralization assay (i.e., a sclerostin neutralizing binding agent).

Antibodies according to the invention may have a binding affinity for human sclerostin of less than or equal to 1×10⁻⁷ M, less than or equal to 1×10⁻⁸ M, less than or equal to 1×10⁻⁹ M, less than or equal to 1×10⁻¹⁰ M, less than or equal to 1×10⁻¹¹ M, or less than or equal to 1×10⁻¹² M.

The affinity of a binding agent such as an antibody or binding partner, as well as the extent to which a binding agent (such as an antibody) inhibits binding, can be determined by one of ordinary skill in the art using conventional techniques, for example, those described by Scatchard et al., Ann. N.Y. Acad. Sci., 51:660-672 (1949)) or by surface plasmon resonance (SPR; BIAcore, Biosensor, Piscataway, N.J.). For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (see, e.g., Wolff et al., Cancer Res., 53:2560-65 (1993)).

An antibody according to the present invention may belong to any immunoglobin class, for example IgG, IgE, IgM, IgD, or IgA. It may be obtained from or derived from an animal, for example, fowl (e.g., chicken) and mammals, which includes but, is not limited, to a mouse, rat, hamster, rabbit, or other rodent, cow, horse, sheep, goat, camel, human, or other primate. The antibody may be an internalizing antibody. Production of antibodies is disclosed generally in U.S. Patent Publication No. 2004/0146888 A1.

### Characterization Assays

In the methods described above to generate antibodies according to the invention, including the manipulation of the specific antibody AA-WW CDRs into new frameworks and/or constant regions, appropriate assays are available to select the desired antibodies or binding agents (i.e., assays for determining binding affinity to sclerostin; cross-blocking assays; Biacore-based “human sclerostin peptide epitope competition binding assay;” MC3T3-E1 cell based assay; and/or in vivo assays).

Cross-Blocking Assays

The terms “cross-block,” “cross-blocked” and “cross-blocking” are used interchangeably herein to mean the ability of an antibody or other binding agent to interfere with the binding of other antibodies or binding agents to sclerostin.

The extent to which an antibody or other binding agent is able to interfere with the binding of another to sclerostin, and therefore whether it can be said to cross-block according to the invention, can be determined using competition binding assays. One particularly suitable quantitative assay uses a Biacore machine which can measure the extent of interactions using surface plasmon resonance technology. Another suitable quantitative cross-blocking assay uses an ELISA-based approach to measure competition between antibodies or other binding agents in terms of their binding to sclerostin.

Biacore Cross-Blocking Assay

The following generally describes a suitable Biacore assay for determining whether an antibody or other binding agent cross-blocks or is capable of cross-blocking according to the invention. For convenience, reference is made to two antibodies, but it will be appreciated that the assay can be used with any of the sclerostin binding agents described herein. The Biacore machine (for example the Biacore 3000) is operated in line with the manufacturer's recommendations.

Thus, in one cross-blocking assay, sclerostin is coupled to a CM5 Biacore chip using standard amine coupling chemistry to generate a sclerostin-coated surface. Typically 200-800 resonance units of sclerostin would be coupled to the chip (an amount that gives easily measurable levels of binding but that is readily saturable by the concentrations of test reagent being used).

The two antibodies (termed 1* and 2*) to be assessed for their ability to cross-block each other are mixed at a one to one molar ratio of binding sites in a suitable buffer to create the test mixture. When calculating the concentrations on a binding site basis the molecular weight of an antibody is assumed to be the total molecular weight of the antibody divided by the number of sclerostin binding sites on that antibody.

The concentration of each antibody in the test mix should be high enough to readily saturate the binding sites for that antibody on the sclerostin molecules captured on the Biacore chip. The antibodies in the mixture are at the same molar concentration (on a binding basis) and that concentration would typically be between 1.00 and 1.5 micromolar (on a binding site basis).

Separate solutions containing antibody 1* alone and antibody 2* alone are also prepared. Antibody 1* and antibody 2* in these solutions should be in the same buffer and at the same concentration as in the test mix.

The test mixture is passed over the sclerostin-coated Biacore chip and the total amount of binding recorded. The chip is then treated in such a way as to remove the bound antibodies without damaging the chip-bound sclerostin. Typically this is done by treating the chip with 30 mM HCl for 60 seconds.

The solution of antibody 1* alone is then passed over the sclerostin-coated surface and the amount of binding recorded. The chip is again treated to remove all of the bound antibody without damaging the chip-bound sclerostin.

The solution of antibody 2* alone is then passed over the sclerostin-coated surface and the amount of binding recorded.

The maximum theoretical binding of the mixture of antibody 1* and antibody 2* is next calculated, and is the sum of the binding of each antibody when passed over the sclerostin surface alone. If the actual recorded binding of the mixture is less than this theoretical maximum then the two antibodies are cross-blocking each other.

Thus, in general, a cross-blocking antibody or other binding agent according to the invention is one which will bind to sclerostin in the above Biacore cross-blocking assay such that during the assay and in the presence of a second antibody or other binding agent of the invention the recorded binding is between 80% and 0.1% (e.g., between 80% and 4%) of the maximum theoretical binding, specifically between 75% and 0.1% (e.g., between 75% and 4%) of the maximum theoretical binding, and more specifically between 70% and 0.1% (e.g., between 70% and 4%) of maximum theoretical binding (as just defined above) of the two antibodies or binding agents in combination.

The Biacore assay described above is a primary assay used to determine if antibodies or other binding agents cross-block each other according to the invention. On rare occasions particular antibodies or other binding agents may not bind to sclerostin coupled via amine chemistry to a CM5 Biacore chip (this usually occurs when the relevant binding site on sclerostin is masked or destroyed by the coupling to the chip). In such cases cross-blocking can be determined using a tagged version of Sclerostin, for example N-terminal His-tagged Sclerostin (R & D Systems, Minneapolis, Minn., USA; 2005 cat#1406-ST-025). In this particular format, an anti-His antibody would be coupled to the Biacore chip and then the His-tagged sclerostin would be passed over the surface of the chip and captured by the anti-H is antibody. The cross blocking analysis would be carried out essentially as described above, except that after each chip regeneration cycle, new His-tagged sclerostin would be loaded back onto the anti-His antibody coated surface. In addition to the example given using N-terminal His-tagged sclerostin, C-terminal His-tagged sclerostin could alternatively be used. Furthermore, various other tags and tag binding protein combinations that are known in the art could be used for such a cross-blocking analysis (e.g., HA tag with anti-HA antibodies; FLAG tag with anti-FLAG antibodies; and/or biotin tag with streptavidin).

### ELISA-Based Cross-Blocking Assay

The following generally describes an ELISA assay for determining whether an anti-sclerostin antibody or other sclerostin binding agent cross-blocks or is capable of cross-blocking according to the invention. For convenience, reference is made to two antibodies (Ab-1 and Ab-2), but it will be appreciated that the assay can be used with any of the sclerostin binding agents described herein.

The general principle of the assay is to have an anti-sclerostin antibody coated onto the wells of an ELISA plate. An excess amount of a second, potentially cross-blocking, anti-sclerostin antibody is added in solution (i.e., not bound to the ELISA plate). A limited amount of sclerostin is then added to the wells. The coated antibody and the antibody in solution compete for binding of the limited number of sclerostin molecules. The plate is washed to remove sclerostin that has not been bound by the coated antibody and to also remove the second, solution phase antibody as well as any complexes formed between the second, solution phase antibody and sclerostin. The amount of bound sclerostin is then measured using an appropriate sclerostin detection reagent. An antibody in solution that is able to cross-block the coated antibody will be able to cause a decrease in the number of sclerostin molecules that the coated antibody can bind relative to the number of sclerostin molecules that the coated antibody can bind in the absence of the second, solution phase, antibody.

This assay is described below in more detail for Ab-1 and Ab-2. In the instance where Ab-1 is chosen to be the immobilized antibody, it is coated onto the wells of the ELISA plate, after which the plates are blocked with a suitable blocking solution to minimize non-specific binding of reagents that are subsequently added. An excess amount of Ab-2 is then added to the ELISA plate such that the moles of Ab-2 sclerostin binding sites per well are at least 10 fold higher than the moles of Ab-1 sclerostin binding sites that were used, per well, during the coating of the ELISA plate. Sclerostin is then added such that the moles of sclerostin added per well are at least 25-fold lower than the moles of Ab-1 sclerostin binding sites that were used for coating each well. Following a suitable incubation period, the ELISA plate is washed and a sclerostin detection reagent is added to measure the amount of sclerostin specifically bound by the coated anti-sclerostin antibody (in this case Ab-1). The background signal for the assay is defined as the signal obtained in wells with the coated antibody (in this case Ab-1), second solution phase antibody (in this case Ab-2), sclerostin buffer only (i.e., no sclerostin) and sclerostin detection reagents. The positive control signal for the assay is defined as the signal obtained in wells with the coated antibody (in this case Ab-1), second solution phase antibody buffer only (i.e., no second solution phase antibody), sclerostin and sclerostin detection reagents. The ELISA assay needs to be run in such a manner so as to have the positive control signal be at least 6 times the background signal.

To avoid any artifacts resulting from the choice of which antibody to use as the coating antibody and which to use as the second (competitor) antibody (e.g., significantly different affinities between Ab-1 and Ab-2 for sclerostin), the cross-blocking assay can be run in two formats:
1) format 1 is where Ab-1 is the antibody that is coated onto the ELISA plate and Ab-2 is the competitor antibody that is in solution and
2) format 2 is where Ab-2 is the antibody that is coated onto the ELISA plate and Ab-1 is the competitor antibody that is in solution.

Ab-1 and Ab-2 are defined as cross-blocking if, either in format 1 or in format 2, the solution phase anti-sclerostin antibody is able to cause a reduction of between 60% and 100%, specifically between 70% and 100%, and more specifically between 80% and 100%, of the sclerostin detection signal (i.e., the amount of sclerostin bound by the coated antibody) as compared to the sclerostin detection signal obtained in the absence of the solution phase anti-sclerostin antibody (i.e., the positive control wells).

### Cell-Based Neutralization Assay

Mineralization by osteoblast-lineage cells in culture, either primary cells or cell lines, is used as an in vitro model of bone formation. Mineralization takes from about one to six weeks to occur beginning with the induction of osteoblast-lineage cell differentiation by one or more differentiation agents. The overall sequence of events involves cell proliferation, differentiation, extracellular matrix production, matrix maturation, and finally deposition of mineral, which refers to crystallization and/or deposition of calcium phosphate. This sequence of events starting with cell proliferation and differentiation, and ending with deposition of mineral, is referred to herein as mineralization. Measurement of calcium (mineral) is the output of the assay.

MC3T3-E1 cells (Sudo et al. “In vitro differentiation and calcification in a new clonal osteogenic cell line derived from newborn mouse calvaria.” J. Cell Biol., 96:191-198 (1983)) and subclones of the original cell line can form mineral in culture upon growth in the presence of differentiating agents. Such subclones include MC3T3-E1-BF (Smith et al., “Glucocorticoids inhibit developmental stage-specific osteoblast cell cycle.” J. Biol. Chem., 275:19992-20001 (2000)). For both the MC3T3-E1-BF subclone as well as the original MC3T3-E1 cells, sclerostin can inhibit one or more of the sequence of events leading up to and including mineral deposition (i.e., sclerostin inhibits mineralization). Anti-sclerostin antibodies that are able to neutralize sclerostin's inhibitory activity allow for mineralization of the culture in the presence of sclerostin such that there is a statistically significant increase in deposition of calcium phosphate (measured as calcium) as compared to the amount of calcium measured in the sclerostin-only (i.e., no antibody) treatment group.

When running the assay with the goal of determining whether a particular anti-sclerostin antibody or anti-sclerostin binding agent can neutralize sclerostin (i.e., is a sclerostin neutralizing antibody or derivative thereof, or is a sclerostin neutralizing binding agent), the amount of sclerostin used in the assay can be the minimum amount of sclerostin that causes at least a 70%, statistically significant, reduction in deposition of calcium phosphate (measured as calcium) in the sclerostin-only group, as compared to the amount of calcium measured in the no sclerostin group. An anti-sclerostin neutralizing antibody or an anti-sclerostin neutralizing binding agent is defined as one that causes a statistically significant increase in deposition of calcium phosphate (measured as calcium) as compared to the amount of calcium measured in the sclerostin-only (i.e., no antibody, no binding agent) treatment group. To determine whether an anti-sclerostin antibody or an anti-sclerostin binding agent is neutralizing or not, the amount of anti-sclerostin antibody or anti-sclerostin binding agent used in the assay needs to be such that there is an excess of moles of sclerostin binding sites per well as compared to the number of moles of sclerostin per well. Depending on the potency of the antibody, the fold excess that may be required can be 24, 18, 12, 6, 3, or 1.5, and one of skill is familiar with the routine practice of testing more than one concentration of binding agent. For example, a very potent anti-sclerostin neutralizing antibody or anti-sclerostin neutralizing binding agent will be able to neutralize sclerostin even when there is less than a 6-fold excess of moles of sclerostin binding sites per well as compared to the number of moles of sclerostin per well. A less potent anti-sclerostin neutralizing antibody or anti-sclerostin neutralizing binding agent will be able to neutralize sclerostin only at a 12, 18, or 24 fold excess. Sclerostin binding agents within this full range of potencies are suitable as neutralizing sclerostin binding agents.

Anti-sclerostin antibodies and derivatives thereof that can neutralize human sclerostin, and sclerostin binding agents that can neutralize human sclerostin, may be of use in the treatment of human conditions/disorders that are caused by, associated with, or result in at least one of low bone formation, low bone mineral density, low bone mineral content, low bone mass, low bone quality and low bone strength.

### In Vivo Neutralization Assay

Increases in various parameters associated with, or that result from, the stimulation of new bone formation can be measured as an output from in vivo testing of sclerostin binding agents in order to identify those binding agents that are able to neutralize sclerostin and thus able to cause stimulation of new bone formation. Such parameters include various serum anabolic markers (e.g., osteocalcin and P1NP (n-terminal propeptide of type 1 procollagen)), histomorphometric markers of bone formation (e.g., osteoblast surface/bone surface; bone formation rate/bone surface; and trabecular thickness), bone mineral density, bone mineral content, bone mass, bone quality, and bone strength. A sclerostin neutralizing binding agent is defined as one capable of causing a statistically significant increase, as compared to vehicle treated animals, in any parameter associated with, or that results from, the stimulation of new bone formation. Such in vivo testing can be performed in any suitable mammal (e.g. mouse, rat, and/or monkey).

Although the amino acid sequence of sclerostin is not 100% identical across mammalian species (e.g., mouse sclerostin is not 100% identical to human sclerostin), it will be appreciated by one skilled in the art that a sclerostin binding agent that can neutralize, in vivo, the sclerostin of a certain species (e.g., mouse) and that also can bind human sclerostin in vitro is very likely to be able to neutralize human sclerostin in vivo. Thus, such a human sclerostin binding agent (e.g., anti-human sclerostin antibody) may be of use in the treatment of human conditions/disorders that are caused by, associated with, or result in at least one of low bone formation, low bone mineral density, low bone mineral content, low bone mass, low bone quality, and low bone strength. Mice in which homologous recombination had been used to delete the mouse sclerostin gene and insert the human sclerostin gene in its place (i.e., human sclerostin gene knock-in mice or human SOST knock-in mice) would be an example of an additional in vivo system.

Pharmaceutical compositions are provided, comprising one of the above-described binding agents such as at least one of antibody AA-WW to human sclerostin, along with a pharmaceutically or physiologically acceptable carrier, excipient, or diluent. Pharmaceutical compositions and methods of treatment are disclosed in copending application Ser. No. 10/868,497, filed Jun. 16, 2004, published as U.S. 2005/0106683, which claims priority to Ser. No. 60/478,977, both of which are incorporated by reference herein.

The development of suitable dosing and treatment regimens for using the particular compositions described herein in a variety of treatment regimens, including e.g., subcutaneous, oral, parenteral, intravenous, intranasal, and intramuscular administration and formulation, is well known in the art, some of which are briefly discussed below for general purposes of illustration.

In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain circumstances it will be desirable to deliver the pharmaceutical compositions disclosed herein subcutaneously, parenterally, intravenously, intramuscularly, or intraperitoneally. Such approaches are well known to the skilled artisan, some of which are further described, for example, in U.S. Pat. No. 5,543,158; U.S. Pat. No. 5,641,515; and U.S. Pat. No. 5,399,363. In certain embodiments, solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations generally will contain a preservative to prevent the growth of microorganisms.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion (see, for example, Remington's Pharmaceutical Sciences, 15th ed., pp. 1035-1038 and 1570-1580). Some variation in dosage will necessarily occur depending on the condition of the subject being treated. Moreover, for human administration, preparations will preferably meet sterility, pyrogenicity, and the general safety and purity standards as required by FDA Office of Biologics standards.

In another embodiment of the invention, the compositions disclosed herein may be formulated in a neutral or salt form. Illustrative pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like. Upon formulation, solutions will be administered in a manner compatible with the dosage formulation and in such amount as is therapeutically effective.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

In certain embodiments, liposomes, nanocapsules, microparticles, lipid particles, vesicles, and the like, are used for the introduction of the compositions of the present invention into suitable host cells/organisms. In particular, the compositions of the present invention may be formulated for delivery either encapsulated in a lipid particle, a liposome, a vesicle, a nanosphere, or a nanoparticle or the like. Alternatively, compositions of the present invention can be bound, either covalently or non-covalently, to the surface of such carrier vehicles.

The formation and use of liposome and liposome-like preparations as potential drug carriers is generally known to those of skill in the art (see for example, Lasic, Trends Biotechnol., 16(7):307-21 (1998); Takakura, Nippon Rinsho, 56(3):691-95 (1998); Chandran et al., Indian J. Exp. Biol., 35(8):801-09 (1997); Margalit, Crit. Rev. Ther. Drug Carrier Syst., 12(2-3):233-61 (1995); U.S. Pat. No. 5,567,434; U.S. Pat. No. 5,552,157; U.S. Pat. No. 5,565,213; U.S. Pat. No. 5,738,868, and U.S. Pat. No. 5,795,587, each specifically incorporated herein by reference in its entirety). The use of liposomes does not appear to be associated with autoimmune responses or unacceptable toxicity after systemic delivery. In certain embodiments, liposomes are formed from phospholipids that are dispersed in an aqueous medium and spontaneously form multilamellar concentric bilayer vesicles (also termed multilamellar vesicles (MLVs)).

Alternatively, in other embodiments, the invention provides for pharmaceutically-acceptable nanocapsule formulations of the compositions of the present invention. Nanocapsules can generally entrap compounds in a stable and reproducible way (see, for example, Quintanar-Guerrero et al., Drug Dev. Ind. Pharm., 24(12):1113-28 (1998)). To avoid side effects due to intracellular polymeric overloading, such ultrafine particles (sized around 0.1 μm) may be designed using polymers able to be degraded in vivo. Such particles can be made as described, for example, by Couvreur et al., Crit. Rev. Ther. Drug Carrier Syst., 5(1):1-20 (1988); zur Muhlen et al., Eur. J. Pharm. Biopharm., 45(2):149-55 (1998); Zambaux et al., J. Controlled Release, 50(1-3):31-40 (1998); and U.S. Pat. No. 5,145,684.

In addition, pharmaceutical compositions of the present invention may be placed within containers, along with packaging material that provides instructions regarding the use of such pharmaceutical compositions. Generally, such instructions will include a tangible expression describing the reagent concentration, as well as within certain embodiments, relative amounts of excipient ingredients or diluents (e.g., water, saline or PBS) that may be necessary to reconstitute the pharmaceutical composition.

The dose administered may range from 0.01 mg/kg to 100 mg/kg of body weight. As will be evident to one of skill in the art, the amount and frequency of administration will depend, of course, on such factors as the nature and severity of the indication being treated, the desired response, the condition of the patient, and so forth. Typically, the compositions may be administered by a variety of techniques, as noted above.

Increases in bone mineral content and/or bone mineral density may be determined directly through the use of X-rays (e.g., Dual Energy X-ray Absorptometry or “DEXA”), or by inference through the measurement of (1) markers of bone formation and/or osteoblast activity, such as, but not limited to, osteoblast specific alkaline phosphatase, osteocalcin, type 1 procollagen C′ propeptide (PICP), total alkaline phosphatase (see Comier, Curr. Opin. in Rheu., 7:243 (1995)) and serum procollagen 1 N-terminal propeptide (P1NP) and/or (2) markers of bone resorption and/or osteoclast activity including, but not limited to, pyridinoline, deoxypyridinoline, N-telopeptide, urinary hydroxyproline, plasma tartrate-resistant acid phosphatases, and galactosyl hydroxylysine (see Comier, id), serum TRAP 5b (tartrate-resistant acid phosphatase isoform 5b) and serum cross-linked C-telopeptide (sCTXI). The amount of bone mass may also be calculated from body weights or by using other methods (see Guinness-Hey, Metab. Bone Dis. Relat. Res., 5:177-181 (1984). Animals and particular animal models are used in the art for testing the effect of the compositions and methods of the invention on, for example, parameters of bone loss, bone resorption, bone formation, bone strength or bone mineralization that mimic conditions of human disease such as osteoporosis and osteopenias. Examples of such models include the ovariectomized rat model (Kalu, D. N., “The ovariectomized rat model of postmenopausal bone loss.” Bone and Mineral, 15:175-192 (1991); Frost, H. M. and Jee, W. S. S., “On the rat model of human osteopenias and osteoporosis.” Bone and Mineral, 18:227-236 (1992); and Jee, W. S. S. and Yao, W., “Overview: animal models of osteopenia and osteoporosis.” J. Musculoskel. Neuron. Interact., 1:193-207 (2001)).

Particular conditions which may be treated by the compositions of the present invention include dysplasias, wherein growth or development of bone is abnormal and a wide variety of causes of osteopenia, osteoporosis, and bone loss. Representative examples of such conditions include achondroplasia, cleidocranial dysostosis, enchondromatosis, fibrous dysplasia, Gaucher's Disease, hypophosphatemic rickets, Marfan's syndrome, multiple hereditary exotoses, neurofibromatosis, osteogenesis imperfecta, osteopetrosis, osteopoikilosis, sclerotic lesions, pseudoarthrosis, and pyogenic osteomyelitis, periodontal disease, anti-epileptic drug induced bone loss, primary and secondary hyperparathyroidism, familial hyperparathyroidism syndromes, weightlessness induced bone loss, osteoporosis (e.g., osteoporosis in men), postmenopausal bone loss, osteoarthritis, renal osteodystrophy, infiltrative disorders of bone, oral bone loss, osteonecrosis of the jaw, juvenile Paget's disease, melorheostosis, metabolic bone diseases, mastocytosis, sickle cell anemia/disease, organ transplant related bone loss, kidney transplant related bone loss, systemic lupus erythematosus, ankylosing spondylitis, epilepsy, juvenile arthritides, thalassemia, mucopolysaccharidoses, fabry disease, Turner syndrome, Down Syndrome, Klinefelter Syndrome, leprosy, Perthes' Disease, adolescent idiopathic scoliosis, infantile onset multi-system inflammatory disease, Winchester Syndrome, Menkes Disease, Wilson's Disease, ischemic bone disease (such as Legg-Calve-Perthes disease, regional migratory osteoporosis), anemic states, conditions caused by steroids, glucocorticoid-induced bone loss, heparin-induced bone loss, bone marrow disorders, scurvy, malnutrition, calcium deficiency, idiopathic osteopenia or osteoporosis, congenital osteopenia or osteoporosis, alcoholism, chronic liver disease, postmenopausal state, chronic inflammatory conditions, rheumatoid arthritis, inflammatory bowel disease, ulcerative colitis, inflammatory colitis, Crohn's disease, oligomenorrhea, amenorrhea, pregnancy, diabetes mellitus, hyperthyroidism, thyroid disorders, parathyroid disorders, Cushing's disease, acromegaly, hypogonadism, immobilization or disuse, reflex sympathetic dystrophy syndrome, regional osteoporosis, osteomalacia, bone loss associated with joint replacement, HIV associated bone loss, bone loss associated with loss of growth hormone, bone loss associated with cystic fibrosis, fibrous dysplasia, chemotherapy associated bone loss, tumor induced bone loss, cancer-related bone loss, hormone ablative bone loss, multiple myeloma, drug-induced bone loss, anorexia nervosa, disease associated facial bone loss, disease associated cranial bone loss, disease associated bone loss of the jaw, disease associated bone loss of the skull, and bone loss associated with space travel. Further conditions relate to bone loss associated with aging, including facial bone loss associated with aging, cranial bone loss associated with aging, jaw bone loss associated with aging, and skull bone loss associated with aging.

Compositions of the present invention may also be useful for improving outcomes in orthopedic procedures, dental procedures, implant surgery, joint replacement, bone grafting, bone cosmetic surgery and bone repair such as fracture healing, nonunion healing, delayed union healing and facial reconstruction. One or more compositions may be administered before, during and/or after the procedure, replacement, graft, surgery or repair.

The invention also provides a diagnostic kit comprising at least one anti-sclerostin binding agent according to the present invention. The binding agent may be an antibody. In addition, such a kit may optionally comprise one or more of the following:
(1) instructions for using the one or more binding agent(s) for screening, diagnosis, prognosis, therapeutic monitoring or any combination of these applications;
(2) a labeled binding partner to the anti-sclerostin binding agent(s);
(3) a solid phase (such as a reagent strip) upon which the anti-sclerostin binding agent(s) is immobilized; and
(4) a label or insert indicating regulatory approval for screening, diagnostic, prognostic or therapeutic use or any combination thereof.
   If no labeled binding partner to the binding agent(s) is provided, the binding agent(s) itself can be labeled with one or more of a detectable marker(s), e.g., a chemiluminescent, enzymatic, fluorescent, or radioactive moiety.

The following examples are offered by way of illustration, and not by way of limitation.

### EXAMPLES

### Example 1

### Recombinant Expression of Sclerostin

Recombinant human sclerostin/SOST is commercially available from R&D Systems (Minneapolis, Minn., USA; 2006 cat#1406-ST-025). Additionally, recombinant mouse sclerostin/SOST is commercially available from R&D Systems (Minneapolis, Minn., USA; 2006 cat#1589-ST-025).

Alternatively, the different species of sclerostin can be expressed transiently in serum-free suspension adapted 293T or 293EBNA cells. Transfections can be performed as 500 mL or 1 L cultures. The following reagents and materials are available from Gibco BRL (now Invitrogen, Carlsbad, Calif.). Catalog numbers are listed in parentheses: serum-free DMEM (21068-028); DMEM/F12 (3:1) (21068/11765); 1× Insulin-Transferrin-Selenium Supplement (51500-056); 1× Pen Strep Glut (10378-016); 2 mM 1-Glutamine (25030-081); 20 mM HEPES (15630-080); 0.01% Pluronic F68 (24040-032). Briefly, the cell inoculum (5.0-10.0×10⁵ cells/mL×culture volume) is centrifuged at 2,500 RPM for 10 minutes at 4° C. to remove the conditioned medium.

The cells are resuspended in serum-free DMEM and centrifuged again at 2,500 RPM for 10 minutes at 4° C. After aspirating the wash solution, the cells are resuspended in growth medium [DMEM/F12 (3:1)+1× Insulin-Transferrin-Selenium Supplement+1× Pen Strep Glut+2 mM L-Glutamine+20 mM HEPES+0.01% Pluronic F68] in a 1 L or 3 L spinner flask culture. The spinner flask culture is maintained on magnetic stir plate at 125 RPM which is placed in a humidified incubator maintained at 37° C. and 5% CO₂. The mammalian expression plasmid DNA (e.g., pcDNA3.1, pCEP4, Invitrogen Life Technologies, Carlsbad, Calif.), containing the complete coding region (and stop codon) of sclerostin with a Kozak consensus sequence (e.g., CCACC) directly 5′ of the start site ATG, is complexed to the transfection reagent in a 50 mL conical tube.

The DNA-transfection reagent complex can be prepared in 5-10% of the final culture volume in serum-free DMEM or OPTI-MEM. The transfection reagents that can be used for this purpose include X-tremeGene RO-1539 (Roche Applied Science, Indianapolis, Ind.), FuGene6 (Roche Applied Science, Indianapolis, Ind.), Lipofectamine 2000 (Invitrogen, Carlsbad, Calif.), and 293fectin (Invitrogen, Carlsbad, Calif.). 1-5 μg plasmid DNA/mL culture is first added to serum-free DMEM, followed by 1-5 μl transfection reagent/mL culture. The complexes can be incubated at room temperature for approximately 10-30 minutes and then added to the cells in the spinner flask. The transfection/expression can be performed for 4-7 days, after which the conditioned medium (CM) is harvested by centrifugation at 4,000 RPM for 60 minutes at 4° C.

### Example 2

### Purification of Recombinant Sclerostin

Recombinant sclerostin was purified from mammalian host cells as follows. All purification processes were carried out at room temperature. One purification scheme was used to purify various species of sclerostin, including murine and human sclerostin. The purification scheme used affinity chromatography followed by cation exchange chromatography.

### Heparin Chromatography

The mammalian host cell conditioned medium (CM) was centrifuged in a Beckman J6-M1 centrifuge at 4000 rpm for 1 hour at 4° C. to remove cell debris. The CM supernatant was then filtered through a sterile 0.2 μm filter. (At this point the sterile filtered CM may be optionally stored frozen until purification.) If the CM was frozen, it was thawed at the following temperatures, or combination thereof: 4° C., room temperature or warm water. Following thawing, the CM was filtered through a sterile 0.2 μm filter and optionally concentrated by tangential flow ultrafiltration (TFF) using a 10 kD molecular weight cut-off membrane. The CM concentrate was filtered through a sterile 0.2 μm filter and then loaded onto a Heparin High Performance (Heparin HP) column (GE Healthcare, formerly Amersham Biosciences) equilibrated in PBS. Alternatively, the filtered CM supernatant may be loaded directly onto the Heparin HP column equilibrated in PBS.

After loading, the Heparin HP column was washed with PBS until the absorbance at 280 nm of the flow-through returned to baseline (i.e., absorbance measured before loading CM supernatant). The sclerostin was then eluted from the column using a linear gradient from 150 mM to 2M sodium chloride in PBS. The absorbance at 280 nm of the eluate was monitored and fractions containing protein were collected. The fractions were then assayed by Coomassie-stained SDS-PAGE to identify fractions containing a polypeptide that migrates at the size of glycosylated sclerostin. The appropriate fractions from the column were combined to make the Heparin HP pool.

### Cation Exchange Chromatography

The sclerostin eluted from the Heparin HP column was further purified by cation exchange chromatography using SP High Performance (SPHP) chromatography media (GE Healthcare, formerly Amersham Biosciences). The Heparin HP pool was buffer exchanged into PBS by dialysis using 10,000 MWCO membranes (Pierce Slide-A-Lyzer). The dialyzed Heparin HP pool was then loaded onto an SPHP column equilibrated in PBS. After loading, the column was washed with PBS until the absorbance at 280 nm of the flow-through returned to baseline. The sclerostin was then eluted from the SPHP column using a linear gradient from 150 mM to 1 M sodium chloride in PBS. The absorbance at 280 nm of the eluate was monitored and the eluted sclerostin was collected in fractions. The fractions were then assayed by Coomassie-stained SDS-PAGE to identify fractions containing a polypeptide that migrates at the size of glycosylated sclerostin. The appropriate fractions from the column were combined to make the SPHP pool.

### Formulation

Following purification, the SPHP pool was formulated in PBS by dialysis using 10,000 MWCO membranes (Pierce Slide-A-Lyzer). If concentration of sclerostin was necessary, a centrifugal device (Amicon Centricon or Centriprep) with a 10,000 MWCO membrane was used. Following formulation the sclerostin was filtered through a sterile 0.2 μm filter and stored at 4° C. or frozen.

### Example 3

### ELISA-Based Cross-Blocking Assay

An antibody is coated on an ELISA plate at 2 μg/ml. While plates are blocking, the test antibody is incubated with human sclerostin at a final concentration of 25 ng/ml for one hour at room temperature in a separate plate. This complex is then transferred to the blocked ELISA plate and incubated for a further one hour at room temperature. Plates are washed and a pool of biotinylated anti-sclerostin antibodies at 1 ug/ml is then added and incubated for one hour at room temperature. Plates are then washed and streptavidin-horseradish peroxidase conjugate added at a 1:5000 dilution. Plates are developed with TMB. Blocking antibodies are able to reduce the ELISA signal due to inhibition of sclerostin binding to the coated antibodies. Positive crossblocking wells are considered to be those wells which decreased the signal by at least 40%.

### Example 4

### ELISA-Based Cross-Blocking Assay

Liquid volumes used in this example would be those typically used in 96-well plate ELISAs (e.g. 50-200 μl/well). Ab-X and Ab-Y, in this example are assumed to have molecular weights of about 145 Kd and to have 2 sclerostin binding sites per antibody molecule. An anti-sclerostin antibody (Ab-X) is coated (e.g. 50μ of 1 μg/ml) onto a 96-well ELISA plate [e.g. Corning 96 Well EIA/RIA Flat Bottom Microplate (Product #3590), Corning Inc., Acton, Mass.] for at least one hour. After this coating step the antibody solution is removed, the plate is washed once or twice with wash solution (e.g., PBS and 0.05% Tween 20) and is then blocked using an appropriate blocking solution (e.g., PBS, 1% BSA, 1% goat serum and 0.5% Tween 20) and procedures known in the art. Blocking solution is then removed from the ELISA plate and a second anti-sclerostin antibody (Ab-Y), which is being tested for it's ability to cross-block the coated antibody, is added in excess (e.g. 50 μl of 10 μg/ml) in blocking solution to the appropriate wells of the ELISA plate. Following this, a limited amount (e.g. 50 μl of 10 ng/ml) of sclerostin in blocking solution is then added to the appropriate wells and the plate is incubated for at least one hour at room temperature while shaking. The plate is then washed 2-4 times with wash solution. An appropriate amount of a sclerostin detection reagent [e.g., biotinylated anti-sclerostin polyclonal antibody that has been pre-complexed with an appropriate amount of a streptavidin-horseradish peroxidase (HRP) conjugate] in blocking solution is added to the ELISA plate and incubated for at least one hour at room temperature. The plate is then washed at least 4 times with wash solution and is developed with an appropriate reagent [e.g. HRP substrates such as TMB (calorimetric) or various HRP luminescent substrates]. The background signal for the assay is defined as the signal obtained in wells with the coated antibody (in this case Ab-X), second solution phase antibody (in this case Ab-Y), sclerostin buffer only (i.e. no sclerostin) and sclerostin detection reagents. The positive control signal for the assay is defined as the signal obtained in wells with the coated antibody (in this case Ab-X), second solution phase antibody buffer only (i.e. no second solution phase antibody), sclerostin and sclerostin detection reagents. The ELISA assay needs to be run in such a manner so as to have the positive control signal be at least 6 times the background signal.

To avoid any artifacts (e.g. significantly different affinities between Ab-X and Ab-Y for sclerostin) resulting from the choice of which antibody to use as the coating antibody and which to use as the second (competitor) antibody, the cross-blocking assay needs to be run in two formats:

1) format 1 is where Ab-X is the antibody that is coated onto the ELISA plate and Ab-Y is the competitor antibody that is in solution and

2) format 2 is where Ab-Y is the antibody that is coated onto the ELISA plate and Ab-X is the competitor antibody that is in solution.

Ab-X and Ab-Y are defined as cross-blocking if, either in format 1 or in format 2, the solution phase anti-sclerostin antibody is able to cause a reduction of between 60% and 100%, specifically between 70% and 100%, and more specifically between 80% and 100%, of the sclerostin detection signal (i.e. the amount of sclerostin bound by the coated antibody) as compared to the sclerostin detection signal obtained in the absence of the solution phase anti-sclerostin antibody (i.e. the positive control wells).

In the event that a tagged version of sclerostin is used in the ELISA, such as a N-terminal His-tagged Sclerostin (R&D Systems, Minneapolis, Minn., USA; 2005 cat#1406-ST-025) then an appropriate type of sclerostin detection reagent would include an HRP labeled anti-His antibody. In addition to using N-terminal His-tagged Sclerostin, one could also use C-terminal His-tagged Sclerostin. Furthermore, various other tags and tag binding protein combinations that are known in the art could be used in this ELISA-based cross-blocking assay (e.g., HA tag with anti-HA antibodies; FLAG tag with anti-FLAG antibodies; biotin tag with streptavidin).

From the foregoing, although specific embodiments of the invention have been described herein for purposes of illustration, various modifications may be made without deviating from the spirit and scope of the invention. Accordingly, the invention is not limited except as by the appended claims. All publications, published patent applications, and patent documents disclosed herein are hereby incorporated by reference.

1.-34. (canceled)

35. A polypeptide that cross-blocks the binding of at least one of antibodies Antibody AA-Antibody WW to human sclerostin.

36. The polypeptide of claim 35, comprising one or more complementarity determining regions (CDRs) of any one of Antibody AA-Antibody WW, wherein the polypeptide comprises a binding affinity for human sclerostin of less than or equal to 1×10⁻⁷ M.

37. The polypeptide of claim 36, comprising three or more CDRs of any one of Antibody AA-Antibody WW.

38. The polypeptide of claim 37, comprising six CDRs of any one of Antibody AA-Antibody WW.

39. The polypeptide of claim 36, wherein the polypeptide comprises one or more CDR amino acid sequences selected from the group consisting of SEQ ID NOs:101, 104, 110, 116, 122, 128, 134, 140, 146, 152, 158, 164, 170, 176, 182, 188, 194, 200, 206, 212, 218, 224, 230; SEQ ID NO:102, 105, 111, 117, 123, 129, 135, 141, 147, 153, 159, 165, 171, 177, 183, 189, 195, 201, 207, 213, 219, 225, 231; SEQ ID NO:103, 106, 112, 118, 124, 130, 136, 142, 148, 154, 160, 166, 172, 178, 184, 190, 196, 202, 208, 214, 220, 226, 232; SEQ ID NO: 98, 107, 113, 119, 125, 131, 137, 143, 149, 155, 161, 167, 173, 179, 185, 191, 197, 203, 209, 215, 221, 227, 233; SEQ ID NO:99, 108, 114, 120, 132, 144, 150, 156, 162, 168, 174, 180, 186, 192, 198, 204, 210, 216, 222, 228, 234; SEQ ID NO:100, 109, 115, 121, 127, 133, 139, 145, 151, 157, 163, 169, 175, 181, 187, 193, 199, 205, 211, 217, 223, 229, and 235.

40. The polypeptide of claim 36, wherein the polypeptide comprises one or more CDR amino acid sequences selected from the group consisting of SEQ ID NOs:98, 99, 100, 104, 105, 106, 110, 111, 112, 116, 117, 118, 122, 123, 124, 128, 129, 130, 134, 135, 136, 140, 141, 142, 146, 147, 148, 152, 153, 154, 158, 159, 160, 164, 165, 166, 170, 171, 172, 176, 177, 178, 182, 183, 184, 188, 189, 190, 194, 195, 196, 200, 201, 202, 206, 207, 208, 212, 213, 214, 218, 219, 220, 224, 225, 226, 230, 231 and 232.

41. The polypeptide of claim 37, comprising:
(a) CDR sequences of SEQ ID NOs: 98, 99, and 100;
(b) CDR sequences of SEQ ID NOs: 101, 102, and 103;
(c) CDR sequences of SEQ ID NOs: 104, 105, and 106;
(d) CDR sequences of SEQ ID NOs: 107, 108, and 109;
(e) CDR sequences of SEQ ID NOs: 110, 111, and 112;
(f) CDR sequences of SEQ ID NOs: 113, 114, and 115;
(g) CDR sequences of SEQ ID NOs: 116, 117, and 118;
(h) CDR sequences of SEQ ID NOs: 119, 120, and 121;
(i) CDR sequences of SEQ ID NOs: 122, 123, and 124
(j) CDR sequences of SEQ ID NOs: 125, 126, and 127;
(k) CDR sequences of SEQ ID NOs: 128, 129, and 130;
(l) CDR sequences of SEQ ID NOs: 131, 132, and 133;
(m) CDR sequences of SEQ ID NOs: 134, 135, and 136;
(n) CDR sequences of SEQ ID NOs: 137, 138, and 139;
(o) CDR sequences of SEQ ID NOs: 140, 141, and 142;
(p) CDR sequences of SEQ ID NOs: 143, 144, and 145;
(q) CDR sequences of SEQ ID NOs: 146, 147, and 148;
(r) CDR sequences of SEQ ID NOs: 149, 150, and 151;
(s) CDR sequences of SEQ ID NOs: 152, 153, and 154;
(t) CDR sequences of SEQ ID NOs: 155, 156, and 157;
(u) CDR sequences of SEQ ID NOs: 158, 159, and 160;
(v) CDR sequences of SEQ ID NOs: 161, 162, and 163;
(w) CDR sequences of SEQ ID NOs: 164, 165, and 166;
(x) CDR sequences of SEQ ID NOs: 167, 168, and 169;
(y) CDR sequences of SEQ ID NOs: 170, 171, and 172;
(z) CDR sequences of SEQ ID NOs: 173, 174, and 175;
(aa) CDR sequences of SEQ ID NOs: 176, 177, and 178;
(bb) CDR sequences of SEQ ID NOs: 179, 180, and 181;
(cc) CDR sequences of SEQ ID NOs: 182, 183, and 184;
(dd) CDR sequences of SEQ ID NOs: 185, 186, and 187;
(ee) CDR sequences of SEQ ID NOs: 188, 189, and 190;
(ff) CDR sequences of SEQ ID NOs: 191, 192, and 193;
(gg) CDR sequences of SEQ ID NOs: 194, 195, and 196;
(hh) CDR sequences of SEQ ID NOs: 197, 198, and 199;
(ii) CDR sequences of SEQ ID NOs: 200, 201, and 202;
(jj) CDR sequences of SEQ ID NOs: 203, 204, and 205;
(kk) CDR sequences of SEQ ID NOs: 206, 207, and 208;
(ll) CDR sequences of SEQ ID NOs: 209, 210, and 211;
(mm) CDR sequences of SEQ ID NOs: 212, 213, and 214;
(nn) CDR sequences of SEQ ID NOs: 215, 216, and 217;
(oo) CDR sequences of SEQ ID NOs: 218, 219, and 220;
(pp) CDR sequences of SEQ ID NOs: 221, 222, and 223;
(qq) CDR sequences of SEQ ID NOs: 224, 225, and 226;
(rr) CDR sequences of SEQ ID NOs: 227, 228, and 229;
(ss) CDR sequences of SEQ ID NOs: 230, 231, and 232; or
(tt) CDR sequences of SEQ ID NOs: 233, 234, and 235;

42. The polypeptide of claim 41, comprising:
(a) CDR sequences of SEQ ID NOs: 98, 99, and 100 and CDR sequences of SEQ ID NOs: 101, 102, and 103;
(b) CDR sequences of SEQ ID NOs: 104, 105, and 106 and CDR sequences of SEQ ID NOs: 107, 108, and 109;
(c) CDR sequences of SEQ ID NOs: 110, 111, and 112 and CDR sequences of SEQ ID NOs: 113, 114, and 115;
(d) CDR sequences of SEQ ID NOs: 116, 117, and 118 and CDR sequences of SEQ ID NOs: 119, 120, and 121;
(e) CDR sequences of SEQ ID NOs: 122, 123, and 124 and CDR sequences of SEQ ID NOs: 125, 126, and 127;
(f) CDR sequences of SEQ ID NOs: 128, 129, and 130 and CDR sequences of SEQ ID NOs: 131, 132, and 133;
(g) CDR sequences of SEQ ID NOs: 134, 135, and 136 and CDR sequences of SEQ ID NOs: 137, 138, and 139;
(h) CDR sequences of SEQ ID NOs: 140, 141, and 142 and CDR sequences of SEQ ID NOs: 143, 144, and 145;
(i) CDR sequences of SEQ ID NOs: 146, 147, and 148 and CDR sequences of SEQ ID NOs: 149, 150, and 151;
(j) CDR sequences of SEQ ID NOs: 152, 153, and 154 and CDR sequences of SEQ ID NOs: 155, 156, and 157;
(k) CDR sequences of SEQ ID NOs: 158, 159, and 160 and CDR sequences of SEQ ID NOs: 161, 162, and 163;
(l) CDR sequences of SEQ ID NOs: 164, 165, and 166 and CDR sequences of SEQ ID NOs: 167, 168, and 169;
(m) CDR sequences of SEQ ID NOs: 170, 171, and 172 and CDR sequences of SEQ ID NOs: 173, 174, and 175;
(n) CDR sequences of SEQ ID NOs: 176, 177, and 178 and CDR sequences of SEQ ID NOs: 179, 180, and 181;
(o) CDR sequences of SEQ ID NOs: 182, 183, and 184 and CDR sequences of SEQ ID NOs: 185, 186, and 187;
(p) CDR sequences of SEQ ID NOs: 188, 189, and 190 and CDR sequences of SEQ ID NOs: 191, 192, and 193;
(q) CDR sequences of SEQ ID NOs: 194, 195, and 196 and CDR sequences of SEQ ID NOs: 197, 198, and 199;
(r) CDR sequences of SEQ ID NOs: 200, 201, and 202 and CDR sequences of SEQ ID NOs: 203, 204, and 205;
(s) CDR sequences of SEQ ID NOs: 206, 207, and 208 and CDR sequences of SEQ ID NOs: 209, 210, and 211;
(t) CDR sequences of SEQ ID NOs: 212, 213, and 214 and CDR sequences of SEQ ID NOs: 215, 216, and 217;
(u) CDR sequences of SEQ ID NOs: 218, 219, and 220 and CDR sequences of SEQ ID NOs: 221, 222, and 223;
(v) CDR sequences of SEQ ID NOs: 224, 225, and 226 and CDR sequences of SEQ ID NOs: 227, 228, and 229; or
(w) CDR sequences of SEQ ID NOs: 230, 231, and 232 and CDR sequences of SEQ ID NOs: 233, 234, and 235.

43. The polypeptide of claim 36 comprising the amino acid sequence of SEQ ID NO: 16, 18, 44, 46, and/or 47.

44. The polypeptide of claim 36, wherein the polypeptide comprises a binding affinity for human sclerostin of less than or equal to 1×10⁻⁸ M.

45. The polypeptide of claim 44, wherein the polypeptide comprises a binding affinity for human sclerostin of less than or equal to 1×10⁻⁹ M.

46. An isolated nucleic acid molecule comprising a polynucleotide encoding the polypeptide of claim 36.

47. A vector comprising the isolated nucleic acid molecule of claim 46.

48. A host cell comprising the vector of claim 47.

49. A method of making a protein comprising culturing a host cell of claim 48 under conditions wherein the encoded protein is expressed.

50. An isolated antibody selected from the group consisting of Antibody AA-Antibody WW.
